SEQUENCE LISTING (27 sequences)

SEQ ID NO: 1 - protein, 4116 aa, Homo sapiens
  misc_feature: Q8TD57
MGATGRLELTLAAPPHPGPAFQRSKARETQGEEEGSEMQIAKSDSIHHMSHSQGQPELPPLPASANEEPSGLYQTVMSHSFYPPLMQRTSWTLAAPFKEQHHHRGPSDSIANNYSLMAQDLKLKDLLKVYQPATISVPRDRTGQGLPSSGNRSSSEPMRKKTKFSSRNKEDSTRIKLAFKTSIFSPMKKEVKTSLTFPGSRPMSPEQQLDVMLQQEMEMESKEKKPSESDLERYYYYLTNGIRKDMIAPEEGEVMVRISKLISNTLLTSPFLEPLMVVLVQEKENDYYCSLMKSIVDYILMDPMERKRLFIESIPRLFPQRVIRAPVPWHSVYRSAKKWNEEHLHTVNPMMLRLKELWFAEFRDLRFVRTAEILAGKLPLQPQEFWDVIQKHCLEAHQTLLNKWIPTCAQLFTSRKEHWIHFAPKSNYDSSRNIEEYFASVASFMSLQLRELVIKSLEDLVSLFMIHKDGNDFKEPYQEMKFFIPQLIMIKLEVSEPIIVFNPSFDGCWELIRDSFLEIIKNSNGIPKLKYIPLKFSFTAAAADRQCVKAAEPGEPSMHAAATAMAELKGYNLLLGTVNAEEKLVSDFLIQTFKVFQKNQVGPCKYLNVYKKYVDLLDNTAEQNIAAFLKENHDIDDFVTKINAIKKRRNEIASMNITVPLAMFCLDATALNHDLCERAQNLKDHLIQFQVDVNRDTNTSICNQYSHIADKVSEVPANTKELVSLIEFLKKSSAVTVFKLRRQLRDASERLEFLMDYADLPYQIEDIFDNSRNLLLHKRDQAEMDLIKRCSEFELRLEGYHRELESFRKREVMTTEEMKHNVEKLNELSKNLNRAFAEFELINKEEELLEKEKSTYPLLQAMLKNKVPYEQLWSTAYEFSIKSEEWMNGPLFLLNAEQIAEEIGNMWRTTYKLIKTLSDVPAPRRLAENVKIKIDKFKQYIPILSISCNPGMKDRHWQQISEIVGYEIKPTETTCLSNMLEFGFGKFVEKLEPIGAAASKEYSLEKNLDRMKLDWVNVTFSFVKYRDTDTNILCAIDDIQMLLDDHVIKTQTMCGSPFIKPIEAECRKWEEKLIRIQDNLDAWLKCQATWLYLEPIFSSEDIIAQMPEEGRKFGIVDSYWKSLMSQAVKDNRILVAADQPRMAEKLQEANFLLEDIQKGLNDYLEKKRLFFPRFFFLSNDELLEILSETKDPLRVQPHLKKCFEGIAKLEFTDNLEIVGMISSEKETVPFIQKIYPANAKGMVEKWLQQVEQMMLASMREVIGLGIEAYVKVPRNHWVLQWPGQVVICVSSIFWTQEVSQALAENTLLDFLKKSNDQIAQIVQLVRGKLSSGARLTLGALTVIDVHARDVVAKLSEDRVSDLNDFQWISQLRYYWVAKDVQVQIITTEALYGYEYLGNSPRLVITPLTDRCYRTLMGALKLNLGGAPEGPAGTGKTETTKDLAKALAKQCVVFNCSDGLDYKAMGKFFKGLAQAGAWACFDEFNRIEVEVLSVVAQQILSIQQAIIRKLKTFIFEGTELSLNPTCAVFITMNPGYAGRAELPDNLKALFRTVAMMVPDYALIGEISLYSMGFLDSRSLAQKIVATYRLCSEQLSSQHHYDYGMRAVKSVLTAAGNLKLKYPEENESVLLLRALLDVNLAKFLAQDVPLFQGIISDLFPGVVLPKPDYEVFLKVLNDNIKKMKLQPVPWFIGKIIQIYEMMLVRHGYMIVGDPMGGKTSAYKVLAAALGDLHAANQMEEFAVEYKIINPKAITMGQLYGCFDQVSHEWMDGVLANAFREQASSLSDDRKWIIFDGPVDAIWIENMNTVLDDNKKLCLMSGEIIQMNSKMSLIFEPADLEQASPATVSRCGMIYMEPHQLGWKPLKDSYMDTLPSSLTKEHKELVNDMFMWLVQPCLEFGRLHCKFVVQTSPIHLAFSMMRLYSSLLDEIRAVEEEEMELGEGLSSQQIFLWLQGLFLFSLVWTVAGTINADSRKKFDVFFRNLIMGMDDNHPRPKSVKLTKNNIFPERGSIYDFYFIKQASGHWETWTQYITKEEEKVPAGAKVSELIIPTMETARQSFFLKTYLDHEIPMLFVGPTGTGKSAITNNFLLHLPKNTYLPNCINFSARTSANQTQDIIMSKLDRRRKGLFGPPIGKKAVVFVDDLNMPAKEVYGAQPPIELLRQWIDHGYWFDKKDTTRLDIVDMLLVTAMGPPGGGRNDITGRFTRHLNIISINAFEDDILTKIFSSIVDWHFGKGFDVMFLRYGKMLVQATKTIYRDAVENFLPTPSKSHYVFNLRDFSRVIQGVLLCPHTHLQDVEKCIRLWIHEVYRVFYDRLIDKEDRQVFFNMVKETTSNCFKQTIEKVLIHLSPTGKIVDDNIRSLFFGDYFKPESDQKIYDEITDLKQLTVVMEHYLEEFNNISKAPMSLVMFRFAIEHISRICRVLKQDKGHLLLVGIGGSGRQSAAKLSTFMNAYELYQIEITKNYAGNDWREDLKKIILQVGVATKSTVFLFADNQIKDESFVEDINMLLNTGDVPNIFPADEKADIVEKMQTAARTQGEKVEVTPLSMYNFFIERVINKISFSLAMSPIGDAFRNRLRMFPSLINCCTIDWFQSWPTDALELVANKFLEDVELDDNIRVEVVSMCKYFQESVKKLSLDYYNKLRRHNYVTPTSYLELILTFKTLLNSKRQEVAMMRNRYLTGLQKLDFAASQVAVMQRELTALQPQLILTSEETAKMMVKIEAETREADGKKLLVQADEKEANVAAAIAQGIKNECEGDLAEAMPALEAALAALDTLNPADISLVKSMQNPPGPVKLVMESICIMKGMKPERKPDPSGSGKMIEDYWGVSKKILGDLKFLESLKTYDKDNIPPLTMKRIRERFINHPEFQPAVIKNVSSACEGLCKWVRAMEVYDRVAKVVAPKRERLREAEGKLAAQMQKLNQKRAELKLVVDRLQALNDDFEEMNTKKKDLEENIEICSQKLVRAEKLISGLGGEKDRWTEAARQLGIRYTNLTGDVLLSSGTVAYLGAFTVDYRVQCQNQWLAECKDKVIPGFSDFSLSHTLGDPIKIRAWQIAGLPVDSFSIDNGIIVSNSRRWALMIDPHGQANKWIKNMEKANKLAVIKFSDSNYMRMLENALQLGTPVLIENIGEELDASIEPILLKATFKQQGVEYMRLGENIIEYSRDFKLYITTRLRNPHYLPEVAVKVCLLNFMITPLGLQDQLLGIVAAKEKPELEEKKNQLIVESAKNKKHLKEIEDKILEVLSMSKGNILEDETAIKVLSSSKVLSEEISEKQKVASMTETQIDETRMGYKPVAVHSATIFFCISDLANIEPMYQYSLTWFINLYMHSLTHSTKSEELNLRIKYIIDHFTLSIYNNVCRSLFEKDKLLFSLLLTIGIMKQKKEITEEVWYFLLTGGIALDNPYPNPAPQWLSEKAWAEIVRASALPKLHGLMEHLEQNLGEWKLIYDSAWPHEEQLPGSWKFSQGLEKMVILRCLRPDKMVPAVREFIAEHMGKLYIEAPTFDLQGSYNDSSCCAPLIFVLSPSADPMAGLLKFADDLGMGGTRTQTISLGQGQGPIAAKMINNAIKDGTWVVLQNCHLAASWMPTLEKICEEVIVPESTNARFRLWLTSYPSEKFPVSILQNGIKMTNEPPKGLRANLLRSYLNDPISDPVFFQSCAKAVMWQKMLFGLCFFHAVVQERRNFGPLGWNIPYEFNESDLRISMWQIQMFLNDYKEVPFDALTYLTGECNYGGRVTDDKDRRLLLSLLSMFYCKEIEEDYYSLAPGDTYYIPPHGSYQSYIDYLRNLPITAHPEVFGLHENADITKDNQETNQLFEGVLLTLPRQSGGSGKSPQEVVEELAQDILSKLPRDFDLEEVMKLYPVVYEESMNTVLRQELIRFNRLTKVVRRSLINLGRAIKGQVLMSSELEEVFNSMLVGKVPAMWAAKSYPSLKPLGGYVADLLARLTFFQEWIDKGPPVVFWISGFYFTQSFLTGVSQNYARKYTIPIDHIGFEFEVTPQETVMENNPEDGAYIKGLFLEGARWDRKTMQIGESLPKILYDPLPIIWLKPGESAMFLHQDIYVCPVYKTSARRGTLSTTGHSTNYVLSIELPTDMPQKHWINRGVASLCQLDN

SEQ ID NO: 2 - protein, 504 aa, Homo sapiens
  MISC_FEATURE: Q18PE1
MTEAALVEGQVKLRDGKKWKSRWLVLRKPSPVADCLLMLVYKDKSERIKGLRERSSLTLEDICGLEPGLPYEGLVHTLAIVCLSQAIMLGFDSHEAMCAWDARIRYALGEVHRFHVTVAPGTKLESGPATLHLCNDVLVLARDIPPAVTGQWKLSDLRRYGAVPSGFIFEGGTRCGYWAGVFFLSSAEGEQISFLFDCIVRGISPTKGPFGLRPVLPDPSPPGPSTVEERVAQEALETLQLEKRLSLLSHAGRPGSGGDDRSLSSSSSEASHLDVSASSRLTAWPEQSSSSASTSQEGPRPAAAQAAGEAMVGASRPPPKPLRPRQLQEVGRQSSSDSGIATGSHSSYSSSLSSYAGSSLDVWRATDELGSLLSLPAAGAPEPSLCTCLPGTVEYQVPTSLRAHYDTPRSLCLAPRDHSPPSQGSPGNSAARDSGGQTSAGCPSGWLGTRRRGLVMEAPQGSEATLPGPAPGEPWEAGGPHAGPPPAFFSACPVCGGLKVNPPP

SEQ ID NO: 3 - protein, 326 aa, Homo sapiens
  misc_feature: Q8NFH5
MAAFAVEPQGPALGSEPMMLGSPTSPKPGVNAQFLPGFLMGDLPAPVTPQPRSISGPSVGVMEMRSPLLAGGSPPQPVVPAHKDKSGAPPVRSIYDDISSPGLGSTPLTSRRQPNISVMQSPLVGVTSTPGTGQSMFSPASIGQPRKTTLSPAQLDPFYTQGDSLTSEDHLDDSWVTVFGFPQASASYILLQFAQYGNILKHVMSNTGNWMHIRYQSKLQARKALSKDGRIFGESIMIGVKPCIDKSVMESSDRCALSSPSLAFTPPIKTLGTPTQPGSTPRISTMRPLATAYKASTSDYQVISDRQTPKKDESLVSKAMEYMFGW

SEQ ID NO: 4 - protein, 1049 aa, Homo sapiens
  MISC_FEATURE: Q8WYL5
MALVTLQRSPTPSAASSSASNSELEAGSEEDRKLNLSLSESFFMVKGAALFLQQGSSPQGQRSLQHPHKHAGDLPQHLQVMINLLRCEDRIKLAVRLESAWADRVRYMVVVYSSGRQDTEENILLGVDFSSKESKSCTIGMVLRLWSDTKIHLDGDGGFSVSTAGRMHIFKPVSVQAMWSALQVLHKACEVARRHNYFPGGVALIWATYYESCISSEQSCINEWNAMQDLESTRPDSPALFVDKPTEGERTERLIKAKLRSIMMSQDLENVTSKEIRNELEKQMNCNLKELKEFIDNEMLLILGQMDKPSLIFDHLYLGSEWNASNLEELQGSGVDYILNVTREIDNFFPGLFAYHNIRVYDEETTDLLAHWNEAYHFINKAKRNHSKCLVHCKMGVSRSASTVIAYAMKEFGWPLEKAYNYVKQKRSITRPNAGFMRQLSEYEGILDASKQRHNKLWRQQTDSSLQQPVDDPAGPGDFLPETPDGTPESQLPFLDDAAQPGLGPPLPCCFRRLSDPLLPSPEDETGSLVHLEDPEREALLEEAAPPAEVHRPARQPQQGSGLCEKDVKKKLEFGSPKGRSGSLLQVEETEREEGLGAGRWGQLPTQLDQNLLNSENLNNNSKRSCPNGMEDDAIFGILNKVKPSYKSCADCMYPTASGAPEASRERCEDPNAPAICTQPAFLPHITSSPVAHLASRSRVPEKPASGPTEPPPFLPPAGSRRADTSGPGAGAALEPPASLLEPSRETPKVLPKSLLLKNSHCDKNPPSTEVVIKEESSPKKDMKPAKDLRLLFSNESEKPTTNSYLMQHQESIIQLQKAGLVRKHTKELERLKSVPADPAPPSRDGPASRLEASIPEESQDPAALHELGPLVMPSQAGSDEKSEAAPASLEGGSLKSPPPFFYRLDHTSSFSKDFLKTICYTPTSSSMSSNLTRSSSSDSIHSVRGKPGLVKQRTQEIETRLRLAGLTVSSPLKRSHSLAKLGSLTFSTEDLSSEADPSTVADSQDTTLSESSFLHEPQGTPRDPAATSKPSGKPAPENLKSPSWMSKS

SEQ ID NO: 5 - protein, 2048 aa, Homo sapiens
  MISC_FEATURE: Q8IYD8
MSGRQRTLFQTWGSSISRSSGTPGCSSGTERPQSPGSSKAPLPAAAEAQLESDDDVLLVAAYEAERQLCLENGGFCTSAGALWIYPTNCPVRDYQLHISRAALFCNTLVCLPTGLGKTFIAAVVMYNFYRWFPSGKVVFMAPTKPLVTQQIEACYQVMGIPQSHMAEMTGSTQASTRKEIWCSKRVLFLTPQVMVNDLSRGACPAAEIKCLVIDEAHKALGNYAYCQVVRELVKYTNHFRILALSATPGSDIKAVQQVITNLLIGQIELRSEDSPDILTYSHERKVEKLIVPLGEELAAIQKTYIQILESFARSLIQRNVLMRRDIPNLTKYQIILARDQFRKNPSPNIVGIQQGIIEGEFAICISLYHGYELLQQMGMRSLYFFLCGIMDGTKGMTRSKNELGRNEDFMKLYNHLECMFARTRSTSANGISAIQQGDKNKKFVYSHPKLKKLEEVVIEHFKSWNAENTTEKKRDETRVMIFSSFRDSVQEIAEMLSQHQPIIRVMTFVGHASGKSTKGFTQKEQLEVVKQFRDGGYNTLVSTCVGEEGLDIGEVDLIICFDSQKSPIRLVQRMGRTGRKRQGRIVIILSEGREERIYNQSQSNKRSIYKAISSNRQVLHFYQRSPRMVPDGINPKLHKMFITHGVYEPEKPSRNLQRKSSIFSYRDGMRQSSLKKDWFLSEEEFKLWNRLYRLRDSDEIKEITLPQVQFSSLQNEENKPAQESTTGIHQLSLSEWRLWQDHPLPTHQVDHSDRCRHFIGLMQMIEGMRHEEGECSYELEVESYLQMEDVTSTFIAPRNESNNLASDTFITHKKSSFIKNINQGSSSSVIESDEECAEIVKQTHIKPTKIVSLKKKVSKEIKKDQLKKENNHGIIDSVDNDRNSTVENIFQEDLPNDKRTSDTDEIAATCTINENVIKEPCVLLTECQFTNKSTSSLAGNVLDSGYNSFNDEKSVSSNLFLPFEEELYIVRTDDQFYNCHSLTKEVLANVERFLSYSPPPLSGLSDLEYEIAKGTALENLLFLPCAEHLRSDKCTCLLSHSAVNSQQNLELNSLKCINYPSEKSCLYDIPNDNISDEPSLCDCDVHKHNQNENLVPNNRVQIHRSPAQNLVGENNHDVDNSDLPVLSTDQDESLLLFEDVNTEFDDVSLSPLNSKSESLPVSDKTAISETPLVSQFLISDELLLDNNSELQDQITRDANSFKSRDQRGVQEEKVKNHEDIFDCSRDLFSVTFDLGFCSPDSDDEILEHTSDSNRPLDDLYGRYLEIKEISDANYVSNQALIPRDHSKNFTSGTVIIPSNEDMQNPNYVHLPLSAAKNEELLSPGYSQFSLPVQKKVMSTPLSKSNTLNSFSKIRKEILKTPDSSKEKVNLQRFKEALNSTFDYSEFSLEKSKSSGPMYLHKSCHSVEDGQLLTSNESEDDEIFRRKVKRAKGNVLNSPEDQKNSEVDSPLHAVKKRRFPINRSELSSSDESENFPKPCSQLEDFKVCNGNARRGIKVPKRQSHLKHVARKFLDDEAELSEEDAEYVSSDENDESENEQDSSLLDFLNDETQLSQAINDSEMRAIYMKSLRSPMMNNKYKMIHKTHKNINIFSQIPEQDETYLEDSFCVDEEESCKGQSSEEEVCVDFNLITDDCFANSKKYKTRRAVMLKEMMEQNCAHSKKKLSRIILPDDSSEEENNVNDKRESNIAVNPSTVKKNKQQDHCLNSVPSGSSAQSKVRSTPRVNPLAKQSKQTSLNLKDTISEVSDFKPQNHNEVQSTTPPFTTVDSQKDCRKFPVPQKDGSALEDSSTSGASCSKSRPHLAGTHTSLRLPQEGKGTCILVGGHEITSGLEVISSLRAIHGLQVEVCPLNGCDYIVSNRMVVERRSQSEMLNSVNKNKFIEQIQHLQSMFERICVIVEKDREKTGDTSRMFRRTKSYDSLLTTLIGAGIRILFSSCQEETADLLKELSLVEQRKNVGIHVPTVVNSNKSEALQFYLSIPNISYITALNMCHQFSSVKRMANSSLQEISMYAQVTHQKAEEIYRYIHYVFDIQMLPNDLNQDRLKSDI

SEQ ID NO: 6 - protein, 1257 aa, Homo sapiens
  MISC_FEATURE: O14654
MASCSFTRDQATRRLRGAAAAAAAALAAVVTTPLLSSGTPTALIGTGSSCPGAMWLSTATGSRSDSESEEEDLPVGEEVCKRGYLRKQKHGHRRYFVLKLETADAPARLEYYENARKFRHSVRAAAAAAAAAASGAAIPPLIPPRRVITLYQCFSVSQRADARYRHLIALFTQDEYFAMVAENESEQESWYLLLSRLILESKRRRCGTLGAQPDGEPAALAAAAAAEPPFYKDVWQVIVKPRGLGHRKELSGVFRLCLTDEEVVFVRLNTEVASVVVQLLSIRRCGHSEQYFFLEVGRSTVIGPGELWMQVDDCVVAQNMHELFLEKMRALCADEYRARCRSYSISIGAHLLTLLSARRHLGLVPLEPGGWLRRSRFEQFCHLRAIGDGEDEMLFTRRFVTPSEPVAHSRRGRLHLPRGRRSRRAVSVPASFFRRLAPSPARPRHPAEAPNNGARLSSEVSGSGSGNFGEEGNPQGKEDQEGSGGDYMPMNNWGSGNGRGSGGGQGSNGQGSSSHSSGGNQCSGEGQGSRGGQGSNGQGSGGNQCSRDGQGTAGGHGSGGGQRPGGGHGSGGGQGPGDGHGSGGGKNSGGGKGSGSGKGSDGDGERGKSLKKRSYFGKLTQSKQQQMPPPPPPPPPPPPAGGTGGKGKSGGRFRLYFCVDRGATKECKEAKEVKDAEIPEGAARGPHRARAFDEDEDDPYVPMRPGVATPLVSSSDYMPMAPQNVSASKKRHSRSPFEDSRGYMMMFPRVSPPPAPSPPKAPDTNKEDDSKDNDSESDYMFMAPGAGAIPKNPRNPQGGSSSKSWSSYFSLPNPFRSSPLGQNDNSEYVPMLPGKFLGRGLDKEVSYNWDPKDAASKPSGEGSFSKPGDGGSPSKPSDHEPPKNKAKRPNRLSFITKGYKIKPKPQKPTHEQREADSSSDYVNMDFTKRESNTPAPSTQGLPDSWGIIAEPRQSAFSNYVNVEFGVPFPNPANDLSDLLRAIPRANPLSLDSARWPLPPLPLSATGSNAIEEEGDYIEVIFNSAMTPAMALADSAIRYDAETGRIYVVDPFSECCMDISLSPSRCSEPPPVARLLQEEEQERRRPQSRSQSFFAAARAAVSAFPTDSLERDLSPSSAPAVASAAEPTLALSQVVAAASALAAAPGIGAAAAAAGFDSASARWFQPVANAADAEAVRGAQDVAGGSNPGAHNPSANLARGDNQAGGAAAAAAAPEPPPRSRRVPRPPEREDSDNDDDTHVRMDFARRDNQFDSPKRGR

SEQ ID NO: 7 - protein, 578 aa, Homo sapiens
  MISC_FEATURE: Q96AP4
MLSCNICGETVTSEPDMKAHLIVHMESEIICPFCKLSGVNYDEMCFHIETAHFEQNTLERNFERINTVQYGTSDNKKDNTLQCGMEVNSSILSGCASNHPKNSAQNLTKDSTLKHEGFYSENLTESRKFLKSREKQSSLTEIKGSVYETTYSPPECPFCGKIEEHSEDMETHVKTKHANLLDIPLEDCDQPLYDCPMCGLICTNYHILQEHVDLHLEENSFQQGMDRVQCSGDLQLAHQLQQEEDRKRRSEESRQEIEEFQKLQRQYGLDNSGGYKQQQLRNMEIEVNRGRMPPSEFHRRKADMMESLALGFDDGKTKTSGIIEALHRYYQNAATDVRRVWLSSVVDHFHSSLGDKGWGCGYRNFQMLLSSLLQNDAYNDCLKGMLIPCIPKIQSMIEDAWKEGFDPQGASQLNNRLQGTKAWIGACEVYILLTSLRVKCHIVDFHKSTGPLGTHPRLFEWILNYYSSEGEGSPKVVCTSKPPIYLQHQGHSRTVIGIEEKKNRTLCLLILDPGCPSREMQKLLKQDIEASSLKQLRKSMGNLKHKQYQILAVEGALSLEEKLARRQASQVFTAEKIP

SEQ ID NO: 8 - protein, 2752 aa, Homo sapiens
  MISC_FEATURE: Q9UQ35
MYNGIGLPTPRGSGTNGYVQRNLSLVRGRRGERPDYKGEEELRRLEAALVKRPNPDILDHERKRRVELRCLELEEMMEEQGYEEQQIQEKVATFRLMLLEKDVNPGGKEETPGQRPAVTETHQLAELNEKKNERLRAAFGISDSYVDGSSFDPQRRAREAKQPAPEPPKPYSLVRESSSSRSPTPKQKKKKKKKDRGRRSESSSPRRERKKSSKKKKHRSESESKKRKHRSPTPKSKRKSKDKKRKRSRSTTPAPKSRRAHRSTSADSASSSDTSRSRSRSAAAKTHTTALAGRSPSPASGRRGEGDAPFSEPGTTSTQRPSSPETATKQPSSPYEDKDKDKKEKSATRPSPSPERSSTGPEPPAPTPLLAERHGGSPQPLATTPLSQEPVNPPSEASPTRDRSPPKSPEKLPQSSSSESSPPSPQPTKVSRHASSSPESPKPAPAPGSHREISSSPTSKNRSHGRAKRDKSHSHTPSRRMGRSRSPATAKRGRSRSRTPTKRGHSRSRSPQWRRSRSAQRWGRSRSPQRRGRSRSPQRPGWSRSRNTQRRGRSRSARRGRSHSRSPATRGRSRSRTPARRGRSRSRTPARRRSRSRTPTRRRSRSRTPARRGRSRSRTPARRRSRTRSPVRRRSRSRSPARRSGRSRSRTPARRGRSRSRTPARRGRSRSRTPARRSGRSRSRTPARRGRSRSRTPRRGRSRSRSLVRRGRSHSRTPQRRGRSGSSSERKNKSRTSQRRSRSNSSPEMKKSRISSRRSRSLSSPRSKAKSRLSLRRSLSGSSPCPKQKSQTPPRRSRSGSSQPKAKSRTPPRRSRSSSSPPPKQKSKTPSRQSHSSSSPHPKVKSGTPPRQGSITSPQANEQSVTPQRRSCFESSPDPELKSRTPSRHSCSGSSPPRVKSSTPPRQSPSRSSSPQPKVKAIISPRQRSHSGSSSPSPSRVTSRTTPRRSRSVSPCSNVESRLLPRYSHSGSSSPDTKVKPETPPRQSHSGSISPYPKVKAQTPPGPSLSGSKSPCPQEKSKDSLVQSCPGSLSLCAGVKSSTPPGESYFGVSSLQLKGQSQTSPDHRSDTSSPEVRQSHSESPSLQSKSQTSPKGGRSRSSSPVTELASRSPIRQDRGEFSASPMLKSGMSPEQSRFQSDSSSYPTVDSNSLLGQSRLETAESKEKMALPPQEDATASPPRQKDKFSPFPVQDRPESSLVFKDTLRTPPRERSGAGSSPETKEQNSALPTSSQDEELMEVVEKSEEPAGQILSHLSSELKEMSTSNFESSPEVEERPAVSLTLDQSQSQASLEAVEVPSMASSWGGPHFSPEHKELSNSPLRENSFGSPLEFRNSGPLGTEMNTGFSSEVKEDLNGPFLNQLETDPSLDMKEQSTRSSGHSSSELSPDAVEKAGMSSNQSISSPVLDAVPRTPSRERSSSASSPEMKDGLPRTPSRRSRSGSSPGLRDGSGTPSRHSLSGSSPGMKDIPRTPSRGRSECDSSPEPKALPQTPRPRSRSPSSPELNNKCLTPQRERSGSESSVDQKTVARTPLGQRSRSGSSQELDVKPSASPQERSESDSSPDSKAKTRTPLRQRSRSGSSPEVDSKSRLSPRRSRSGSSPEVKDKPRAAPRAQSGSDSSPEPKAPAPRALPRRSRSGSSSKGRGPSPEGSSSTESSPEHPPKSRTARRGSRSSPEPKTKSRTPPRRRSSRSSPELTRKARLSRRSRSASSSPETRSRTPPRHRRSPSVSSPEPAEKSRSSRRRRSASSPRTKTTSRRGRSPSPKPRGLQRSRSRSRREKTRTTRRRDRSGSSQSTSRRRQRSRSRSRVTRRRRGGSGYHSRSPARQESSRTSSRRRRGRSRTPPTSRKRSRSRTSPAPWKRSRSRASPATHRRSRSRTPLISRRRSRSRTSPVSRRRSRSRTSVTRRRSRSRASPVSRRRSRSRTPPVTRRRSRSRTPTTRRRSRSRTPPVTRRRSRSRTPPVTRRRSRSRTSPITRRRSRSRTSPVTRRRSRSRTSPVTRRRSRSRTSPVTRRRSRSRTPPAIRRRSRSRTPLLPRKRSRSRSPLAIRRRSRSRTPRTARGKRSLTRSPPAIRRRSASGSSSDRSRSATPPATRNHSGSRTPPVALNSSRMSCFSRPSMSPTPLDRCRSPGMLEPLGSSRTPMSVLQQAGGSMMDGPGPRIPDHQRTSVPENHAQSRIALALTAISLGTARPPPSMSAAGLAARMSQVPAPVPLMSLRTAPAANLASRIPAASAAAMNLASARTPAIPTAVNLADSRTPAAAAAMNLASPRTAVAPSAVNLADPRTPTAPAVNLAGARTPAALAALSLTGSGTPPTAANYPSSSRTPQAPASANLVGPRSAHATAPVNIAGSRTAAALAPASLTSARMAPALSGANLTSPRVPLSAYERVSGRTSPPLLDRARSRTPPSAPSQSRMTSERAPSPSSRMGQAPSQSLLPPAQDQPRSPVPSAFSDQSRCLIAQTTPVAGSQSLSSGAVATTTSSAGDHNGMLSVPAPGVPHSDVGEPPASTGAQQPSALAALQPAKERRSSSSSSSSSSSSSSSSSSSSSSSSSGSSSSDSEGSSLPVQPEVALKRVPSPTPAPKEAVREGRPPEPTPAKRKRRSSSSSSSSSSSSSSSSSSSSSSSSSSSSSSSSSSSSSSSSSSPSPAKPGPQALPKPASPKKPPPGERRSRSPRKPIDSLRDSRSLSYSPVERRRPSPQPSPRDQQSSSSERGSRRGQRGDSRSPSHKRRRETPSPRPMRHRSSRSP

SEQ ID NO: 9 - protein, 485 aa, Homo sapiens
  MISC_FEATURE: Q8N6W0
MARLTESEARRQQQQLLQPRPSPVGSSGPEPPGGQPDGMKDLDAIKLFVGQIPRHLDEKDLKPLFEQFGRIYELTVLKDPYTGMHKGCAFLTYCARDSAIKAQTALHEQKTLPGMARPIQVKPADSESRGGRDRKLFVGMLNKQQSEEDVLRLFQPFGVIDECTVLRGPDGSSKGCAFVKFSSHTEAQAAIHALHGSQTMPGASSSLVVKFADTDKERTLRRMQQMVGQLGILTPSLTLPFSPYSAYAQALMQQQTTVLSTSGSYLSPGVAFSPCHIQQIGAVSLNGLPATPIAPASGLHSPPLLGTTAVPGLVAPITNGFAGVVPFPGGHPALETVYANGLVPYPAQSPTVAETLHPAFSGVQQYTAMYPTAAITPIAHSVPQPPPLLQQQQREGPEGCNLFIYHLPQEFGDTELTQMFLPFGNIISSKVFMDRATNQSKCFGFVSFDNPASAQAAIQAMNGFQIGMKRLKVQLKRPKDPGHPY

SEQ ID NO: 10 - protein, 1746 aa, Homo sapiens
  MISC_FEATURE: Q9H792
MSACNTFTEHVWKPGECKNCFKPKSLHQLPPDPEKAPITHGNVKTNANHSNNHRIRNTGNFRPPVAKKPTIAVKPTMIVADGQSICGELSIQEHCENKPVIIGWNRNRAALSQKPLNNNNEDDEGISHVPKPYGNNDSAKKMSDNNNGLTEVLKEIAGLDTAPQIRGNETNSRETFLGRINDCYKRSLERKLPPSCMIGGIKETQGKHVILSGSTEVISNEGGRFCYPEFSSGEESEEDVLFSNMEEEHESWDESDEELLAMEIRMRGQPRFANFRANTLSPVRFFVDKKWNTIPLRNKSLQRICAVDYDDSYDEILNGYEENSVVSYGQGSIQSMVSSDSTSPDSSLTEESRSETASSLSQKICNGGLSPGNPGDSKDMKEIEPNYESPSSNNQDKDSSQASKSSIKVPETHKAVLALRLEEKDGKIAVQTEKEESKASTDVAGQAVTINLVPTEEQAKPYRVVNLEQPLCKPYTVVDVSAAMASEHLEGPVNSPKTKSSSSTPNSPVTSSSLTPGQISAHFQKSSAIRYQEVWTSSTSPRQKIPKVELITSGTGPNVPPRKNCHKSAPTSPTATNISSKTIPVKSPNLSEIKFNSYNNAGMPPFPIIIHDEPTYARSSKNAIKVPIVINPNAYDNLAIYKSFLGTSGELSVKEKTTSVISHTYEEIETESKVPDNTTSKTTDCLQTKGFSNSTEHKRGSVAQKVQEFNNCLNRGQSSPQRSYSSSHSSPAKIQRATQEPVAKIEGTQESQMVGSSSTREKASTVLSQIVASIQPPQSPPETPQSGPKACSVEELYAIPPDADVAKSTPKSTPVRPKSLFTSQPSGEAEAPQTTDSPTTKVQKDPSIKPVTPSPSKLVTSPQSEPPAPFPPPRSTSSPYHAGNLLQRHFTNWTKPTSPTRSTEAESVLHSEGSRRAADAKPKRWISFKSFFRRRKTDEEDDKEKEREKGKLVGLDGTVIHMLPPPPVQRHHWFTEAKGESSEKPAIVFMYRCDPAQGQLSVDQSKARTDQAAVMEKGRAENALLQDSEKKRSHSSPSQIPKKILSHMTHEVTEDFSPRDPRTVVGKQDGRGCTSVTTALSLPELEREDGKEDISDPMDPNPCSATYSNLGQSRAAMIPPKQPRQPKGAVDDAIAFGGKTDQEAPNASQPTPPPLPKKMIIRANTEPISKDLQKSMESSLCVMANPTYDIDPNWDASSAGSSISYELKGLDIESYDSLERPLRKERPVPSAANSISSLTTLSIKDRFSNSMESLSSRRGPSCRQGRGIQKPQRQALYRGLENREEVVGKIRSLHTDALKKLAVKCEDLFMAGQKDQLRFGVDSWSDFRLTSDKPCCEAGDAVYYTASYAKDPLNNYAVKICKSKAKESQQYYHSLAVRQSLAVHFNIQQDCGHFLAEVPNRLLPWEDPDDPEKDEDDMEETEEDAKGETDGKNPKPCSEAASSQKENQGVMSKKQRSHVVVITREVPCLTVADFVRDSLAQHGKSPDLYERQVCLLLLQLCSGLEHLKPYHVTHCDLRLENLLLVHYQPGGTAQGFGPAEPSPTSSYPTRLIVSNFSQAKQKSHLVDPEILRDQSRLAPEIITATQYKKCDEFQTGILIYEMLHLPNPFDENPELKEREYTRADLPRIPFRSPYSRGLQQLASCLLNPNPSERILISDAKGILQCLLWGPREDLFQTFTACPSLVQRNTLLQNWLDIKRTLLMIKFAEKSLDREGGISLEDWLCAQYLAFATTDSLSCIVKILQHR

SEQ ID NO: 11 - protein, 397 aa, Homo sapiens
  MISC_FEATURE: Q9H497
MLRGPWRQLWLFFLLLLPGAPEPRGASRPWEGTDEPGSAWAWPGFQRLQEQLRAAGALSKRYWTLFSCQVWPDDCDEDEEAATGPLGWRLPLLGQRYLDLLTTWYCSFKDCCPRGDCRISNNFTGLEWDLNVRLHGQHLVQQLVLRTVRGYLETPQPEKALALSFHGWSGTGKNFVARMLVENLYRDGLMSDCVRMFIATFHFPHPKYVDLYKEQLMSQIRETQQLCHQTLFIFDEAEKLHPGLLEVLGPHLERRAPEGHRAESPWTIFLFLSNLRGDIINEVVLKLLKAGWSREEITMEHLEPHLQAEIVETIDNGFGHSRLVKENLIDYFIPFLPLEYRHVRLCARDAFLSQELLYKEETLDEIAQMMVYVPKEEQLFSSQGCKSISQRINYFLS

SEQ ID NO: 12 - protein, 115 aa, Homo sapiens
  MISC_FEATURE: Q9UE35
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPP

SEQ ID NO: 13 - protein, 305 aa, Homo sapiens
  MISC_FEATURE: O00743
MAPLDLDKYVEIARLCKYLPENDLKRLCDYVCDLLLEESNVQPVSTPVTVCGDIHGQFYDLCELFRTGGQVPDTNYIFMGDFVDRGYYSLETFTYLLALKAKWPDRITLLRGNHESRQITQVYGFYDECQTKYGNANAWRYCTKVFDMLTVAALIDEQILCVHGGLSPDIKTLDQIRTIERNQEIPHKGAFCDLVWSDPEDVDTWAISPRGAGWLFGAKVTNEFVHINNLKLICRAHQLVHEGYKFMFDEKLVTVWSAPNYCYRCGNIASIMVFKDVNTREPKLFRAVPDSERVIPPRTTTPYFL

SEQ ID NO: 14 - protein, 326 aa, Homo sapiens
  MISC_FEATURE: Q8WXF8
MALSGSTPAPCWEEDECLDYYGMLSLHRMFEVVGGQLTECELELLAFLLDEAPGAAGGLARARSGLELLLELERRGQCDESNLRLLGQLLRVLARHDLLPHLARKRRRPVSPERYSYGTSSSSKRTEGSCRRRRQSSSSANSQQGQWETGSPPTKRQRRSRGRPSGGARRRRRGAPAAPQQQSEPARPSSEGKVTCDIRLRVRAEYCEHGPALEQGVASRRPQALARQLDVFGQATAVLRSRDLGSVVCDIKFSELSYLDAFWGDYLSGALLQALRGVFLTEALREAVGREAVRLLVSVDEADYEAGRRRLLLMEEEGGRRPTEAS

SEQ ID NO: 15 - protein, 684 aa, Homo sapiens
  MISC_FEATURE: P81274
MEENLISMREDHSFHVRYRMEASCLELALEGERLCKSGDCRAGVSFFEAAVQVGTEDLKTLSAIYSQLGNAYFYLHDYAKALEYHHHDLTLARTIGDQLGEAKASGNLGNTLKVLGNFDEAIVCCQRHLDISRELNDKVGEARALYNLGNVYHAKGKSFGCPGPQDVGEFPEEVRDALQAAVDFYEENLSLVTALGDRAAQGRAFGNLGNTHYLLGNFRDAVIAHEQRLLIAKEFGDKAAERRAYSNLGNAYIFLGEFETASEYYKKTLLLARQLKDRAVEAQSCYSLGNTYTLLQDYEKAIDYHLKHLAIAQELNDRIGEGRACWSLGNAYTALGNHDQAMHFAEKHLEISREVGDKSGELTARLNLSDLQMVLGLSYSTNNSIMSENTEIDSSLNGVRPKLGRRHSMENMELMKLTPEKVQNWNSEILAKQKPLIAKPSAKLLFVNRLKGKKYKTNSSTKVLQDASNSIDHRIPNSQRKISADTIGDEGFFDLLSRFQSNRMDDQRCCLQEKNCHTASTTTSSTPPKMMLKTSSVPVVSPNTDEFLDLLASSQSRRLDDQRASFSNLPGLRLTQNSQSVLSHLMTNDNKEADEDFFDILVKCQGSRLDDQRCAPPPATTKGPTVPDEDFFSLILRSQGKRMDEQRVLLQRDQNRDTDFGLKDFLQNNALLEFKNSGKKSADH

SEQ ID NO: 16 - protein, 1087 aa, Homo sapiens
  MISC_FEATURE: Q8NG08
MARSSPYLRQLQGPLLPPRDLVEEDDDYLNDDVEEDEESVFIDAEELCSGGVKAGSLPGCLRVSICDENTQETCKVFGRFPITGAWWRVKVQVKPVVGSRSYQYQVQGFPSYFLQSDMSPPNQKHICALFLKECEVSSDDVNKFLTWVKEVSNYKNLNFENLRETLRTFHKETGRKDQKQPTQNGQEELFLDNEMSLPLENTIPFRNVMTALQFPKIMEFLPVLLPRHFKWIIGSGSKEMLKEIEEILGTHPWKLGFSKITYREWKLLRCEASWIAFCQCESLLQLMTDLEKNALIMYSRLKQICREDGHTYVEVNDLTLTLSNHMSFHAASESLKFLKDIGVVTYEKSCVFPYDLYHAERAIAFSICDLMKKPPWHLCVDVEKVLASIHTTKPENSSDDALNESKPDEVRLENPVDVVDTQDNGDHIWTNGENEINAEISEVQLDQDQVEVPLDRDQVAALEMICSNPVTVISGKGGCGKTTIVSRLFKHIEQLEEREVKKACEDFEQDQNASEEWITFTEQSQLEADKAIEVLLTAPTGKAAGLLRQKTGLHAYTLCQVNYSFYSWTQTMMTTNKPWKFSSVRVLVVDEGSLVSVGIFKSVLNLLCEHSKLSKLIILGDIRQLPSIEPGNLLKDLFETLKSRNCAIELKTNHRAESQLIVDNATRISRRQFPKFDAELNISDNPTLPISIQDKTFIFVRLPEEDASSQSSKTNHHSCLYSAVKTLLQENNLQNAKTSQFIAFRRQDCDLINDCCCKHYTGHLTKDHQSRLVFGIGDKICCTRNAYLSDLLPENISGSQQNNDLDASSEDFSGTLPDFAKNKRDFESNVRLCNGEIFFITNDVTDVTFGKRRSLTINNMAGLEVTVDFKKLMKYCRIKHAWARTIHTFQGSEEQTVVYVVGKAGRQHWQHVYTAVTRGRCRVYVIAEESQLRNAIMKNSFPRKTRLKHFLQSKLSSSGAPPADFPSPRKSSGDSGGPSTPSASPLPVVTDHAMTNDVTWSEASSPDERTLTFAERWQLSSPDGVDTDDDLPKSRASKRTCGVNDDESPSKIFMVGESPQVSSRLQNLRLNNLIPRQLFKPTDNQET

SEQ ID NO: 17 - protein, 1141 aa, Homo sapiens
  MISC_FEATURE: Q96AE7
MAAAVGVRGRYELPPCSGPGWLLSLSALLSVAARGAFATTHWVVTEDGKIQQQVDSPMNLKHPHDLVILMRQEATVNYLKELEKQLVAQKIHIEENEDRDTGLEQRHNKEDPDCIKAKVPLGDLDLYDGTYITLESKDISPEDYIDTESPVPPDPEQPDCTKILELPYSIHAFQHLRGVQERVNLSAPLLPKEDPIFTYLSKRLGRSIDDIGHLIHEGLQKNTSSWVLYNMASFYWRIKNEPYQVVECAMRALHFSSRHNKDIALVNLANVLHRAHFSADAAVVVHAALDDSDFFTSYYTLGNIYAMLGEYNHSVLCYDHALQARPGFEQAIKRKHAVLCQQKLEQKLEAQHRSLQRTLNELKEYQKQHDHYLRQQEILEKHKLIQEEQILRNIIHETQMAKEAQLGNHQICRLVNQQHSLHCQWDQPVRYHRGDIFENVDYVQFGEDSSTSSMMSVNFDVQSNQSDINDSVKSSPVAHSILWIWGRDSDAYRDKQHILWPKRADCTESYPRVPVGGELPTYFLPPENKGLRIHELSSDDYSTEEEAQTPDCSITDFRKSHTLSYLVKELEVRMDLKAKMPDDHARKILLSRINNYTIPEEEIGSFLFHAINKPNAPIWLILNEAGLYWRAVGNSTFAIACLQRALNLAPLQYQDVPLVNLANLLIHYGLHLDATKLLLQALAINSSEPLTFLSLGNAYLALKNISGALEAFRQALKLTTKCPECENSLKLIRCMQFYPFLYNITSSVCSGTVVEESNGSDEMENSDETKMSEEILALVDEFQQAWPLEGFGGALEMKGRRLDLQGIRVLKKGPQDGVARSSCYGDCRSEDDEATEWITFQVKRVKKPKGDHKKTPGKKVETGQIENGHRYQANLEITGPKVASPGPQGKKRDYQRLGWPSPDECLKLRWVELTAIVSTWLAVSSKNIDITEHIDFATPIQQPAMEPLCNGNLPTSMHTLDHLHGVSNRASLHYTGESQLTEVLQNLGKDQYPQQSLEQIGTRIAKVLEKNQTSWVLSSMAALYWRVKGQGKKAIDCLRQALHYAPHQMKDVPLISLANILHNAKLWNDAVIVATMAVEIAPHFAVNHFTLGNVYVAMEEFEKALVWYESTLKLQPEFVPAKNRIQTIQCHLMLKKGRRSP

SEQ ID NO: 18 - protein, 244 aa, Homo sapiens
  MISC_FEATURE: Q9BZM4
MAAAASPAILPRLAILPYLLFDWSGTGRADAHSLWYNFTIIHLPRHGQQWCEVQSQVDQKNFLSYDCGSDKVLSMGHLEEQLYATDAWGKQLEMLREVGQRLRLELADTELEDFTPSGPLTLQVRMSCECEADGYIRGSWQFSFDGRKFLLFDSNNRKWTVVHAGARRMKEKWEKDSGLTTFFKMVSMRDCKSWLRDFLMHRKKRLEPTAPPTMAPGLAQPKAIATTLSPWSFLIILCFILPGI

SEQ ID NO: 19 - protein, 398 aa, Homo sapiens
  MISC_FEATURE: Q5T2D3
MSRKQAAKSRPGSGSRKAEAERKRDERAARRALAKERRNRPESGGGGGCEEEFVSFANQLQALGLKLREVPGDGNCLFRALGDQLEGHSRNHLKHRQETVDYMIKQREDFEPFVEDDIPFEKHVASLAKPGTFAGNDAIVAFARNHQLNVVIHQLNAPLWQIRGTEKSSVRELHIAYRYGEHYDSVRRINDNSEAPAHLQTDFQMLHQDESNKREKIKTKGMDSEDDLRDEVEDAVQKVCNATGCSDFNLIVQNLEAENYNIESAIIAVLRMNQGKRNNAEENLEPSGRVLKQCGPLWEEGGSGARIFGNQGLNEGRTENNKAQASPSEENKANKNQLAKVTNKQRREQQWMEKKKRQEERHRHKALESRGSHRDNNRSEAEANTQVTLVKTFAALNI

SEQ ID NO: 20 - protein, 1001 aa, Homo sapiens
  MISC_FEATURE: Q8IXT5
MAVVIRLLGLPFIAGPVDIRHFFTGLTIPDGGVHIIGGEIGEAFIIFATDEDARRAISRSGGFIKDSSVELFLSSKAEMQKTIEMKRTDRVGRGRPGSGTSGVDSLSNFIESVKEEASNSGYGSSINQDAGFHTNGTGHGNLRPRKTRPLKAENPYLFLRGLPYLVNEDDVRVFFSGLCVDGVIFLKHHDGRNNGDAIVKFASCVDASGGLKCHRSFMGSRFIEVMQGSEQQWIEFGGNAVKEGDVLRRSEEHSPPRGINDRHFRKRSHSKSPRRTRSRSPLGFYVHLKNLSLSIDERDLRNFFRGTDLTDEQIRFLYKDENRTRYAFVMFKTLKDYNTALSLHKTVLQYRPVHIDPISRKQMLKFIARYEKKRSGSLERDRPGHVSQKYSQEGNSGQKLCIYIRNFPFDVTKVEVQKFFADFLLAEDDIYLLYDDKGVGLGEALVKFKSEEQAMKAERLNRRRFLGTEVLLRLISEAQIQEFGVNFSVMSSEKMQARSQSRERGDHSHLFDSKDPPIYSVGAFENFRHQLEDLRQLDNFKHPQRDFRQPDRHPPEDFRHSSEDFRFPPEDFRHSPEDFRRPREEDFRRPSEEDFRRPWEEDFRRPPEDDFRHPREEDWRRPLEEDWRRPLEEDFRRSPTEDFRQLPEEDFRQPPEEDLRWLPEEDFRRPPEEDWRRPPEEDFRRPLQGEWRRPPEDDFRRPPEEDFRHSPEEDFRQSPQEHFRRPPQEHFRRPPPEHFRRPPPEHFRRPPPEHFRRPPPEHFRRPPPEHFRRPPPEHFRRPPQEHFRRPPQEHFRRSREEDFRHPPDEDFRGPPDEDFRHPPDEDFRSPQEEDFRCPSDEDFRQLPEEDLREAPEEDPRLPDNFRPPGEDFRSPPDDFRSHRPFVNFGRPEGGKFDFGKHNMGSFPEGRFMPDPKINCGSGRVTPIKIMNLPFKANVNEILDFFHGYRIIPDSVSIQYNEQGLPTGEAIVAMINYNEAMAAIKDLNDRPVGPRKVKLTLL

SEQ ID NO: 21 - protein, 4427 aa, Homo sapiens
  MISC_FEATURE: Q9P225
MSSKAEKKQRLSGRGSSQASWSGRATRAAVATQEQGNAPAVSEPELQAELPKEEPEPRLEGPQAQSEESVEPEADVKPLFLSRAALTGLADAVWTQEHDAILEHFAQDPTESILTIFIDPCFGLKLELGMPVQTQNQLVYFIRQAPVPITWENFEATVQFGTVRGPYIPALLRLLGGVFAPQIFANTGWPESIRNHFASHLHKFLACLTDTRYKLEGHTVLYIPAEAMNMKPEMVIKDKELVQRLETSMIHWTRQIKEMLSAQETVETGENLGPLEEIEFWRNRCMDLSGISKQLVKKGVKHVESILHLAKSSYLAPFMKLAQQIQDGSRQAQSNLTFLSILKEPYQELAFMKPKDISSKLPKLISLIRIIWVNSPHYNTRERLTSLFRKVCDCQYHFARWEDGKQGPLPCFFGAQGPQITRNLLEIEDIFHKNLHTLRAVRGGILDVKNTCWHEDYNKFRAGIKDLEVMTQNLITSAFELVRDVPHGVLLLDTFHRLASREAIKRTYDKKAVDLYMLFNSELALVNRERNKKWPDLEPYVAQYSGKARWVHILRRRIDRVMTCLAGAHFLPRIGTGKESVHTYQQMVQAIDELVRKTFQEWTSSLDKDCIRRLDTPLLRISQEKAGMLDVNFDKSLLILFAEIDYWERLLFETPHYVVNVAERAEDLRILRENLLLVARDYNRIIAMLSPDEQALFKERIRLLDKKIHPGLKKLHWALKGASAFFITECRIHASKVQMIVNEFKASTLTIGWRAQEMSEKLLVRISGKRVYRDLEFEEDQREHRAAVQQKLMNLHQDVVTIMTNSYEVFKNDGPEIQQQWMLYMIRLDRMMEDALRLNVKWSLLELSKAINGDGKTSPNPLFQVLVILKNDLQGSVAQVEFSPTLQTLAGVVNDIGNHLFSTISVFCHLPDILTKRKLHREPIQTVVEQDEDIKKIQTQISSGMTNNASLLQNYLKTWDMYREIWEINKDSFIHRYQRLNPPVSSFVADIARYTEVANNVQKEETVTNIQFVLLDCSHLKFSLVQHCNEWQNKFATLLREMAAGRLLELHTYLKENAEKISRPPQTLEELGVSLQLVDALKHDLANVETQIPPIHEQFAILEKYEVPVEDSVLEMLDSLNGEWVVFQQTLLDSKQMLKKHKEKFKTGLIHSADDFKKKAHTLLEDFEFKGHFTSNVGYMSALDQITQVRAMLMAMREEENSLRANLGIFKIEQPPSKDLQNLEKELDALQQIWEIARDWEENWNEWKTGRFLILQTETMETTAHGLFRRLTKLAKEYKDRNWEIIETTRSKIEQFKRTMPLISDLRNPALRERHWDQVRDEIQREFDQESESFTLEQIVELGMDQHVEKIGEISASATKELAIEVALQNIAKTWDVTQLDIVPYKDKGHHRLRGTEEVFQALEDNQVALSTMKASRFVKAFEKDVDHWERCLSLILEVIEMILTVQRQWMYLENIFLGEDIRKQLPNESTLFDQVNSNWKAIMDRMNKDNNALRSTHHPGLLDTLIEMNTILEDIQKSLDMYLETKRHIFPRFYFLSNDDLLEILGQSRNPEAVQPHLKKCFDNIKLLRIQKVGGPSSKWEAVGMFSGDGEYIDFLHSVFLEGPVESWLGDVEQTMRVTLRDLLRNCHLALRKFLNKRDKWVKEWAGQVVITASQIQWTADVTKCLLTAKERADKKILKVMKKNQVSILNKYSEAIRGNLTKIMRLKIVALVTIEIHARDVLEKLYKSGLMDVNSFDWLSQLRFYWEKDLDDCVIRQTNTQFQYNYEYLGNSGRLVITPLTDRCYMTLTTALHLHRGGSPKGPAGTGKTETVKDLGKALGIYVIVVNCSEGLDYKSMGRMYSGLAQTGAWGCFDEFNRINIEVLSVVAHQILCILSALAAGLTHFHFDGFEINLVWSCGIFITMNPGYAGRTELPENLKSMFRPIAMVVPDSTLIAEIILFGEGFGNCKILAKKVYTLYSLAVQQLSRQDHYDFGLRALTSLLRYAGKKRRLQPDLTDEEVLLLSMRDMNIAKLTSVDAPLFNAIVQDLFPNIELPVIDYGKLRETVEQEIRDMGLQSTPFTLTKVFQLYETKNSRHSTMIVGCTGSGKTASWRILQASLSSLCRAGDPNFNIVREFPLNPKALSLGELYGEYDLSTNEWTDGILSSVMRTACADEKPDEKWILFDGPVDTLWIENMNSVMDDNKVLTLINGERIAMPEQVSLLFEVEDLAMASPATVSRCGMVYTDYADLGWKPYVQSWLEKRPKAEVEPLQRMFEKLINKMLAFKKDNCKELVPLPEYSGITSLCKLYSALATPENGVNPADGENYVTMVEMTFVFSMIWSVCASVDEEGRKRIDSYLREIEGSFPNKDTVYEYFVDPKIRSWTSFEDKLPKSWRYPPNAPFYKIMVPTVDTVRYNYLVSSLVANQNPILLVGPVGTGKTSIAQSVLQSLPSSQWSVLVVNMSAQTTSNNVQSIIESRVEKRTKGVYVPFGGKSMITFMDDLNMPAKDMFGSQPPLELIRLWIDYGFWYDRTKQTIKYIREMFLMAAMGPPGGGRTVISPRLRSRFNIINMTFPTKSQIIRIFGTMINQKLQDFEEEVKPIGNVVTEATLDMYNTVVQRFLPTPTKMHYLFNLRDISKVFQGMLRANKDFHDTKSSITRLWIHECFRVFSDRLVDAADTEAFMGIISDKLGSFFDLTFHHLCPSKRPPIFGDFLKEPKVYEDLTDLTVLKTVMETALNEYNLSPSVVPMQLVLFREAIEHITRIVRVIGQPRGNMLLVGIGGSGRQSLARLASSICDYTTFQIEVTKHYRKQEFRDDIKRLYRQAGVELKTTSFIFVDTQIADESFLEDINNILSSGEVPNLYKPDEFEEIQSHIIDQARVEQVPESSDSLFAYLIERVQNNLHIVLCLSPMGDPFRNWIRQYPALVNCTTINWFSEWPQEALLEVAEKCLIGVDLGTQENIHRKVAQIFVTMHWSVAQYSQKMLLELRRHNYVTPTKYLELLSGYKKLLGEKRQELLAQANKLRTGLFKIDETREKVQVMSLELEDAKKKVAEFQKQCEEYLVIIVQQKREADEQQKAVTANSEKIAVEEIKCQALADNAQKDLEEALPALEEAMRALESLNKKDIGEIKSYGRPPAQVEIVMQAVMILRGNEPTWAEAKRQLGEQNFIKSLINFDKDNISDKVLKKIGAYCAQPDFQPDIIGRVSLAAKSLCMWVRAMELYGRLYRVVEPKRIRMNAALAQLREKQAALAEAQEKLREVAEKLEMLKKQYDEKLAQKEELRKKSEEMELKLERAGMLVSGLAGEKARWEETVQGLEEDLGYLVGDCLLAAAFLSYMGPFLTNYRDEIVNQIWIGKIWELQVPCSPSFAIDNFLCNPTKVRDWNIQGLPSDAFSTENGIIVTRGNRWALMIDPQAQALKWIKNMEGGQGLKIIDLQMSDYLRILEHAIHFGYPVLLQNVQEYLDPTLNPMLNKSVARIGGRLLMRIGDKEVEYNTNFRFYITTKLSNPHYSPETSAKTTIVNFAVKEQGLEAQLLGIVVRKERPELEEQKDSLVINIAAGKRKLKELEDEILRLLNEATGSLLDDVQLVNTLHTSKITATEVTEQLETSETTEINTDLAREAYRPCAQRASILFFVLNDMGCIDPMYQFSLDAYISLFILSIDKSHRSNKLEDRIDYLNDYHTYAVYRYTCRTLFERHKLLFSFHMCAKILETSGKLNMDEYNFFLRGGVVLDREGQMDNPCSSWLADAYWDNITELDKLTNFHGLMNSFEQYPRDWHLWYTNAAPEKAMLPGEWENACNEMQRMLIVRSLRQDRVAFCVTSFIITNLGSRFIEPPVLNMKSVLEDSTPRSPLVFILSPGVDPTSALLQLAEHMGMAQRFHALSLGQGQAPIAARLLREGVTQGHWVFLANCHLSLSWMPNLDKLVEQLQVEDPHPSFRLWLSSIPHPDFPISILQVSIKMTTEPPKGLKANMTRLYQLMSEPQFSRCSKPAKYKKLLFSLCFFHSVLLERKKFLQLGWNIIYGFNDSDFEVSENLLSLYLDEYEETPWDALKYLIAGINYGGHVTDDWDRRLLTTYINDYFCDQSLSTPFHRLSALETYFIPKDGSLASYKEYISLLPGMDPPEAFGQHPNADVASQITEAQTLFDTLLSLQPQITPTRAGGQTREEKVLELAADVKQKIPEMIDYEGTQKLLALDPSPLNVVLLQEIQRYNTLMQTILFSLTDLEKGIQGLIVMSTSLEEIFNCIFDAHVPPLWGKAYPSQKPLAAWTRDLAMRVEQFELWASRARPPVIFWLSGFTFPTGFLTAVLQSSARQNNVSVDSLSWEFIVSTVDDSNLVYPPKDGVWVRGLYLEGAGWDRKNSCLVEAEPMQLVCLMPTIHFRPAESRKKSAKGMYSCPCYYYPNRAGSSDRASFVIGIDLRSGAMTPDHWIKRGTALLMSLDS

SEQ ID NO: 22 - protein, 924 aa, Homo sapiens
  MISC_FEATURE: Q9Y2I9
MDVLPTGGGRPGLRTELEFRGGGGEARLESQEEETIPAAPPAPRLRGAAERPRRSRDTWDGDEDTEPGEACGGRTSRTASLVSGLLNELYSCTEEEEAAGGGRGAEGRRRRRDSLDSSTEASGSDVVLGGRSGAGDSRVLQELQERPSQRHQMLYLRQKDANELKTILRELKYRIGIQSAKLLRHLKQKDRLLHKVQRNCDIVTACLQAVSQKRRVDTKLKFTLEPSLGQNGFQQWYDALKAVARLSTGIPKEWRRKVWLTLADHYLHSIAIDWDKTMRFTFNERSNPDDDSMGIQIVKDLHRTGCSSYCGQEAEQDRVVLKRVLLAYARWNKTVGYCQGFNILAALILEVMEGNEGDALKIMIYLIDKVLPESYFVNNLRALSVDMAVFRDLLRMKLPELSQHLDTLQRTANKESGGGYEPPLTNVFTMQWFLTLFATCLPNQTVLKIWDSVFFEGSEIILRVSLAIWAKLGEQIECCETADEFYSTMGRLTQEMLENDLLQSHELMQTVYSMAPFPFPQLAELREKYTYNITPFPATVKPTSVSGRHSKARDSDEENDPDDEDAVVNAVGCLGPFSGFLAPELQKYQKQIKEPNEEQSLRSNNIAELSPGAINSCRSEYHAAFNSMMMERMTTDINALKRQYSRIKKKQQQQVHQVYIRADKGPVTSILPSQVNSSPVINHLLLGKKMKMTNRAAKNAVIHIPGHTGGKISPVPYEDLKTKLNSPWRTHIRVHKKNMPRTKSHPGCGDTVGLIDEQNEASKTNGLGAAEAFPSGCTATAGREGSSPEGSTRRTIEGQSPEPVFGDADVDVSAVQAKLGALELNQRDAAAETELRVHPPCQRHCPEPPSAPEENKATSKAPQGSNSKTPIFSPFPSVKPLRKSATARNLGLYGPTERTPTVHFPQMSRSFSKPGGGNSGTKKR

SEQ ID NO: 23 - protein, 190 aa, Homo sapiens
  MISC_FEATURE: P41222 (PTGDS)
MATHHTLWMGLALLGVLGDLQAAPEAQVSVQPNFQQDKFLGRWFSAGLASNSSWLREKKAALSMCKSVVAPATDGGLNLTSTFLRKNQCETRTMLLQPAGSLGSYSYRSPHWGSTYSVSVVETDYDQYALLYSQGSKGPGEDFRMATLYSRTQTPRAELKEKFTAFCKAQGFTEDTIVFLPQTDKCMTEQ

SEQ ID NO: 24 - protein, 372 aa, Homo sapiens [sequence omitted]

SEQ ID NO: 25 - protein, 518 aa, Homo sapiens
  MISC_FEATURE: Q06418 (TYRO3)
TVEGTRANLTGWDPQKDLIVRVCVSNAVGCGPWSQPLVVSSHDRAGQQGPPHSRTSWVPVVLGVLTALVTAAALALILLRKRRKETRFGQAFDSVMARGEPAVHFRAARSFNRERPERIEATLDSLGISDELKEKLEDVLIPEQQFTLGRMLGKGEFGSVREAQLKQEDGSFVKVAVKMLKADIIASSDIEEFLREAACMKEFDHPHVAKLVGVSLRSRAKGRLPIPMVILPFMKHGDLHAFLLASRIGENPFNLPLQTLIRFMVDIACGMEYLSSRNFIHRDLAARNCMLAEDMTVCVADFGLSRKIYSGDYYRQGCASKLPVKWLALESLADNLYTVQSDVWAFGVTMWEIMTRGQTPYAGIENAEIYNYLIGGNRLKQPPECMEDVYDLMYQCWSADPKQRPSFTCLRMELENILGQLSVLSASQDPLYINIERAEEPTAGGSLELPGRDQPYSGAGDGSGMGAVGGTPSDCRYILTPGGLAEQPGQAEHQPESPLNETQRLLLLQQGLLPHSSC

SEQ ID NO: 26 - protein, 607 aa, Homo sapiens
  MISC_FEATURE: P52306 (RAP1GDS1)
MDNLSDTLKKLKITAVDKTEDSLEGCLDCLLQALAQNNTETSEKIQASGILQLFASLLTPQSSCKAKVANIIAEVAKNEFMRIPCVDAGLISPLVQLLNSKDQEVLLQTGRALGNICYDSHEGRSAVDQAGGAQIVIDHLRSLCSITDPANEKLLTVFCGMLMNYSNENDSLQAQLINMGVIPTLVKLLGIHCQNAALTEMCLVAFGNLAELESSKEQFASTNIAEELVKLFKKQIEHDKREMIFEVLAPLAENDAIKLQLVEAGLVECLLEIVQQKVDSDKEDDITELKTGSDLMVLLLLGDESMQKLFEGGKGSVFQRVLSWIPSNNHQLQLAGALAIANFARNDANCIHMVDNGIVEKLMDLLDRHVEDGNVTVQHAALSALRNLAIPVINKAKMLSAGVTEAVLKFLKSEMPPVQFKLLGTLRMLIDAQAEAAEQLGKNVKLVERLVEWCEAKDHAGVMGESNRLLSALIRHSKSKDVIKTIVQSGGIKHLVTMATSEHVIMQNEALVALALIAALELGTAEKDLESAKLVQILHRLLADERSAPEIKYNSMVLICALMGSECLHKEVQDLAFLDVVSKLRSHENKSVAQQASLTEQRLTVES

SEQ ID NO: 27 - protein, 322 aa, Homo sapiens
  MISC_FEATURE: Q9Y5Y7 (LYVE1)
MARCFSLVLLLTSIWTTRLLVQGSLRAEELSIQVSCRIMGITLVSKKANQQLNFTEAKEACRLLGLSLAGKDQVETALKASFETCSYGWVGDGFVVISRISPNPKCGKNGVGVLIWKVPVSRQFAAYCYNSSDTWTNSCIPEIITTKDPIFNTQTATQTTEFIVSDSTYSVASPYSTIPAPTTTPPAPASTSIPRRKKLICVTEVFMETSTMSTETEPFVENKAAFKNEAAGFGGVPTALLVLALLFFGAAAGLGFCYVKRYVKAFPFTNKNQQKEMIETKVVKEEKANDSNPNEESKKTDKNPEESKSPSKTTVRCLEAEV

(19) United States
(12) Patent Application Publication
(10) Pub. No.: US 2016/0223569 A1
(43) Pub. Date: Aug. 4, 2016

(54) NOVEL NON-INVASIVE METHODS OF MONITORING HIV VIRAL LOADS

(71) Applicant: DREXEL UNIVERSITY, Philadelphia, PA (US)

(72) Inventors: Hans Peter SCHLECHT, Philadelphia, PA (US); Keith VOSSELLER, Philadelphia, PA (US)

(21) Appl. No.: 15/021,963

(22) PCT Filed: Sep. 2, 2014
(86) PCT No.: PCT/US14/53676

§ 371 Date: Mar. 15, 2016

Related U.S. Application Data
(60) Provisional application No. 61/881,767, filed on Sep. 24, 2013.

(51) Int. Cl. G01N 33/68; G01N 33/569

(52) U.S. Cl. CPC G01N 33/6893; G01N 33/56988; G01N 2333/16; G01N 2469/10; G01N 2560/00

(57) ABSTRACT

The present invention provides a method of assessing or monitoring systemic HIV viral load in an HIV-infected human patient. In certain embodiments, the method comprises analyzing a sample comprising urine from the patient for the presence and/or concentration of at least one protein selected from a specific group of proteins.

### CROSS-REFERENCE TO RELATED APPLICATIONS

The present application claims priority under 35 U.S.C. §119(e) to U.S. Provisional Patent Application No. 61/881,767, filed Sep. 24, 2013, which application is incorporated herein by reference in its entirety.

### STATEMENT REGARDING FEDERALLY SPONSORED RESEARCH OR DEVELOPMENT

This invention was made with government support under 1R03AI083149-01A1 and 5R03AI083149-02 awarded by National Institute of Allergy and Infectious Diseases (National Institutes of Health). The government has certain rights in the invention.

### BACKGROUND OF THE INVENTION

Viral load testing (i.e., measuring the number of copies of HIV in the blood) is the only way to accurately assess the level of viral replication in HIV-infected patients. Routine monitoring of viral load helps reinforce a patient's adherence to anti-retroviral therapy (ART), thereby ensuring viral suppression and preventing treatment failure before it occurs. Routine testing also ensures that health care workers can diagnose treatment failure early on when drug resistance occurs, and appropriately switch patients from first-line ART to more effective second-line treatment regimens. With large numbers of patients throughout the world already on treatment for several years, ensuring patients can be tested for viral load is a global priority. Furthermore, viral load monitoring is a critical component of programs that aim to reduce transmission rates.

For patients on ART, the World Health Organization (WHO) recommends viral load testing twice yearly. Unfortunately, viral load testing remains largely unavailable in resource-limited settings, in which the majority of HIV-infected patients reside. Viral load testing is rarely available or convenient in poor countries, resulting in avoidable morbidity and mortality and increasing the risk of transmission of drug-resistant forms of the virus.

It is thus critical that access to viral load testing in resource-limited settings be prioritized as part of the fight against HIV/AIDS. Current viral load tests are fairly complex, requiring specialized laboratory facilities. Unfortunately, the majority of HIV-infected patients rely on points of service without reliable power supply or highly trained staff. In such cases, transport of samples to central reference laboratories is unfeasible and/or cost-prohibitive. Further, a lack of market competition for viral load testing kits results in high testing costs. Simple tests that can be performed at a community-based clinics, and/or a point-of-care test that can be performed at a point of service, are now urgently needed throughout the world.

There is a need in the art for novel convenient and effective methods of identifying and/or monitoring patients with (un)controlled HIV infection. Such methods may be used to determine whether the patient is responding to anti-retroviral therapy. The present invention fulfills this need.

### BRIEF SUMMARY OF THE INVENTION

The invention includes a method of assessing or monitoring systemic HIV viral load in an HIV-infected human patient. The invention also includes a kit for assessing or monitoring systemic HIV viral load in an HIV-infected human patient.

In certain embodiments, the method includes analyzing a test sample comprising urine from the patient for the presence or concentration of at least one protein, whereby a test data set is obtained.

In certain embodiments, the methods includes comparing the test data set with a control data set relating to the presence or concentration of the at least one protein in a control sample.

In certain embodiments, the methods allows for assessing and/or monitoring the HIV viral load in the patient.

In certain embodiments, the patient has received or is receiving a first anti-HIV medication. In other embodiments, the patient is a new-born human or an infant younger than about 18 months of age.

In certain embodiments, the test sample is prepared by a method comprising subjecting urine from the patient to at least one procedure selected from the group consisting of protein isolation and protein digestion. In other embodiments, the test sample is analyzed using mass spectrometry, a quantum dot assay or a chromophore assay. In yet other embodiments, the test sample is analyzed using a method comprising contacting the test sample with an antibody or aptamer. In yet other embodiments, the antibody is at least one selected from the group consisting of a polyclonal antibody, monoclonal antibody, Fv, Fab, F(ab)2, single chain antibody, human antibody, humanized antibody, and fragments and derivatives thereof. In yet other embodiments, the antibody or aptamer is used in an immunoassay. In yet other embodiments, the immunoassay comprises at least one selected from the group consisting of immunoturbidimetry, immunonephelometry, ELISA assay, radioimmunoassay, chemiluminescence immunoassay, immunofluorescence, immunoprecipitation, immunoelectrophoresis, and flow cytometry-based immunoassay.

In certain embodiments, the control sample comprises an urine sample from an untreated HIV-infected control human. In other embodiments, the untreated HIV-infected control human is the human patient before receiving anti-HIV medication. In yet other embodiments, the control sample comprises an urine sample from an HIV-uninfected control human. In yet other embodiments, the control sample comprises an urine sample from an HIV-infected control human with controlled infection.

In certain embodiments, the concentration of the protein in the patient's urine is higher by at least a multiplicity factor than the concentration of the protein in the urine from an HIV-uninfected control human or from an HIV-infected control human with controlled infection, wherein the patient is identified as having uncontrolled HIV infection, whereby the patient is prescribed a second anti-HIV medication that is distinct from the first anti-HIV medication. In other embodiments, the multiplicity factor is selected from the group consisting of about 1.1, 1.2, 1.3, 1.4, 1.5, 1.6, 1.7. 1.8, 1.9, 2, 2.25, 2.5, 2.75, 3, 3.5, 4, 5, 6, 7, 8, 9, 10, 11, 12, 13, 14, 15, 20, 30, 40, 50, 75, 100, 125, 250, 500 and 1,000.

In certain embodiments, the concentration of the protein in the patient's urine is lower by at least a multiplicity factor than the concentration of the protein in the urine from an HIV-uninfected control human or from an HIV-infected control human with controlled infection, wherein the patient is identified as having controlled HIV infection, whereby the patient continues to be prescribed the first anti-HIV medication. In other embodiments, the multiplicity factor is selected from the group consisting of about 1.1, 1.2, 1.3, 1.4, 1.5, 1.6, 1.7. 1.8, 1.9, 2, 2.25, 2.5, 2.75, 3, 3.5, 4, 5, 6, 7, 8, 9, 10, 11, 12, 13, 14, 15, 20, 30, 40, 50, 75, 100, 125, 250, 500 and 1,000.

In certain embodiments, the concentration of the protein in the patient's urine is equal to or greater than a multiplicity factor of the concentration of the protein in the urine from an untreated HIV-positive control human, wherein the patient is identified as having uncontrolled HIV infection, whereby the patient is prescribed a second anti-HIV medication which is distinct from the first anti-HIV medication. In other embodiments, the multiplicity factor is selected from the group consisting of about 1, 0.95, 0.9, 0.8, 0.7, 0.6, 0.5, 0.4, 0.3, 0.2, 0.1, 0.05, 0.025, 0.01, 0.005, 0.0025, 0.001, 0.0005, 0.00025, 0.0001, 0.00005 and 0.00001.

In certain embodiments, the concentration of the protein in the patient's sample is lower than a multiplicity factor of the concentration of the protein in the urine from an untreated HIV-positive control human, wherein the patient is identified as having controlled HIV infection, whereby the patient continues to be prescribed the first anti-HIV medication. In other embodiments, the multiplicity factor is selected from the group consisting of about 0.95, 0.9, 0.8, 0.7, 0.6, 0.5, 0.4, 0.3, 0.2, 0.1, 0.05, 0.025, 0.01, 0.005, 0.0025, 0.001, 0.0005, 0.00025, 0.0001, 0.00005 and 0.00001.

In certain embodiments, the at least one protein has an accession number selected from the group consisting of Q8TD57 (SEQ ID NO:1), Q18PE1 (SEQ ID NO:2), Q8NFH5 (SEQ ID NO:3), Q8WYL5 (SEQ ID NO:4), Q8IYD8 (SEQ ID NO:5), O14654 (SEQ ID NO:6), Q96AP4 (SEQ ID NO:7), Q9UQ35 (SEQ ID NO:8), Q8N6W0 (SEQ ID NO:9), Q9H792 (SEQ ID NO:10), Q9H497 (SEQ ID NO:11), Q9UE35 (SEQ ID NO:12), 000743 (SEQ ID NO:13), Q8WXF8 (SEQ ID NO:14), P81274 (SEQ ID NO:15), Q8NG08 (SEQ ID NO:16), Q96AE7 (SEQ ID NO:17), Q9BZM4 (SEQ ID NO:18), Q5T2D3 (SEQ ID NO:19), Q8IXT5 (SEQ ID NO:20), Q9P225 (SEQ ID NO:21), and Q9Y2I9 (SEQ ID NO:22).

In certain embodiments, the at least one protein has an accession number selected from the group consisting of P41222 (PTGDS) (SEQ ID NO:23), P14151 (SELL) (SEQ ID NO:24), Q06418 (TYRO3) (SEQ ID NO:25), P52306 (RAP1GDS1) (SEQ ID NO:26), and Q9Y5Y7 (LYVE1) (SEQ ID NO:27).

In certain embodiments, the kit includes an antibody or aptamer that binds to at least one protein with an accession number selected from the group consisting of Q8TD57 (SEQ ID NO:1), Q18PE1 (SEQ ID NO:2), Q8NFH5 (SEQ ID NO:3), Q8WYL5 (SEQ ID NO:4), Q8IYD8 (SEQ ID NO:5), O14654 (SEQ ID NO:6), Q96AP4 (SEQ ID NO:7), Q9UQ35 (SEQ ID NO:8), Q8N6W0 (SEQ ID NO:9), Q9H792 (SEQ ID NO:10), Q9H497 (SEQ ID NO:11), Q9UE35 (SEQ ID NO:12), O00743 (SEQ ID NO:13), Q8WXF8 (SEQ ID NO:14), P81274 (SEQ ID NO:15), Q8NG08 (SEQ ID NO:16), Q96AE7 (SEQ ID NO:17), Q9BZM4 (SEQ ID NO:18), Q5T2D3 (SEQ ID NO:19), Q8IXT5 (SEQ ID NO:20), Q9P225 (SEQ ID NO:21), and Q9Y2I9 (SEQ ID NO:22).

In certain embodiments, the kit includes an antibody or aptamer that binds to at least one protein with an accession number selected from the group consisting of P41222 (PTGDS) (SEQ ID NO:23), P14151 (SELL) (SEQ ID NO:24), Q06418 (TYRO3) (SEQ ID NO:25), P52306 (RAP1GDS1) (SEQ ID NO:26), and Q9Y5Y7 (LYVE1) (SEQ ID NO:27).

In certain embodiments, the kit includes an applicator. In other embodiments, the kit includes an instructional material for the use of the kit. In yet other embodiments, the instruction material comprises instructions for analyzing a test sample comprising urine from the patient for the presence or concentration of the at least one protein.

In certain embodiments, the kit further comprises a test data set with a control data set relating to the presence or concentration of the at least one protein in a control sample. In other embodiments, the control sample comprises an urine sample from an untreated HIV-infected control human. In yet other embodiments, the control sample comprises an urine sample from an HIV-uninfected control human. In yet other embodiments, the control sample comprises an urine sample from an HIV-infected control human with controlled infection.

### BRIEF DESCRIPTION OF THE DRAWINGS

The following detailed description of specific embodiments of the invention will be better understood when read in conjunction with the appended drawings. For the purpose of illustrating the invention, there are shown in the drawings specific embodiments. It should be understood, however, that the invention is not limited to the precise arrangements and instrumentalities of the embodiments shown in the drawings.

FIG. 1 is a table illustrating characteristics of the study population in Example 1.

FIG. 2, comprising FIGS. 2A-2B, is a table illustrating a selected list of proteins identified in the urine of HIV-infected patients in Example 1.

FIG. 3, comprising FIGS. 3A-3F, is a table illustrating a selected list of proteins identified in the urine of HIV-infected patients in Example 2. Highlighted are proteins that are unique to HIV urine proteomes compared to non-HIV urine, as well as proteins that display greatly increased abundance in HIV urine proteomes compared to non-HIV urine. Relative abundance is reflected in the columns displaying spectral counts for each peptide/protein identified.

### DETAILED DESCRIPTION OF THE INVENTION

The present invention relates to the unexpected discovery of a novel, non-invasive method for monitoring or assessing HIV viral load in a human. The method comprises analyzing an urine sample from the human for the presence and/or concentration of one or more protein markers that are associated with active systemic HIV replication. The method allows for the monitoring or assessment of systemic HIV replication and/or infection in a human and the identification of a human with uncontrolled HIV infection.

In certain embodiments, change in the urinary proteome, as compared to the urinary proteome of an untreated HIV-infected control human or a HIV-uninfected control human, correlates with systemic HIV replication. In other embodiments, change in the urinary proteome, as compared to the urinary proteome of an untreated HIV-infected control human or an HIV-uninfected control human, acts as a surrogate for serum HIV viral load. In yet other embodiments, the urine proteome of an HIV-infected human with high serum viral loads (such as, but not limited to, equal to or greater than about 1,000 copies/mL) can be distinguished from the urine proteome of an HIV-infected human with low serum viral loads (such as, but not limited to, equal to or less than about 200 copies/mL, or equal to or less than 400 copies/mL).

In one aspect, the method of the invention allows for HIV treatment monitoring using a rapid point-of-care urine test. In certain embodiments, the human has been or is being administered highly active antiretroviral therapy (HAART). In other embodiments, the human has uncontrolled HIV infection. In yet other embodiments, the human has controlled HIV infection.

As disclosed herein, the urinary proteome in subjects with uncontrolled HIV infection was analyzed using mass spectrometry. In certain embodiments, analysis of the urine samples identified thousands of peptides corresponding to human-unique proteins. Although no HIV proteins were detected, several host proteins were found exclusively in the urine of patients infected with HIV as compared to published surveys of the non-HIV-infected human urinary proteome. In certain embodiments, these HIV-specific proteomic signatures provide insights into the human physiological response to HIV infection and serve as novel HIV biomarkers in urine.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

The term “about” as used herein, when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the term “acceptable carrier” means an acceptable material, composition or carrier, such as a liquid or solid filler, stabilizer, dispersing agent, suspending agent, diluent, excipient, thickening agent, solvent or encapsulating material, involved in carrying or transporting a compound useful in the methods of the invention such that it may perform its intended function. Each carrier must be “acceptable” in the sense of being compatible with the other compounds useful in the methods of the invention, and not interfering with the method of the invention. Some examples of materials that may serve as acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other compatible substances.

As used herein, “acceptable carrier” also includes any and all coatings, antibacterial and antifungal agents, and absorption delaying agents, and the like that are compatible with the activity of the compound useful in the methods of the invention. Supplementary active compounds may also be incorporated into the compositions. Other additional ingredients that may be included in the compositions used in the practice of the invention are known in the art and described, for example in Remington's Pharmaceutical Sciences (Genaro, Ed., Mack Publishing Co., 1985, Easton, Pa.), which is incorporated herein by reference.

The term “antibody” as used herein refers to an immunoglobulin molecule that specifically binds with an antigen. An antibody of the invention includes intracellularly expressed antibody, or intrabody. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies, human antibodies, and humanized antibodies (Harlow, et al., 1999, In: Using Antibodies: A Laboratory Manual, Cold Spring Harbor Laboratory Press, NY; Harlow, et al., 1989, In: Antibodies: A Laboratory Manual, Cold Spring Harbor, N.Y.; Houston, et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)₂, and Fv fragments, linear antibodies, scFv antibodies, and multispecific antibodies formed from antibody fragments.

An “antibody heavy chain” as used herein refers to the larger of the two types of polypeptide chains present in all antibody molecules in their naturally occurring conformations.

An “antibody light chain” as used herein refers to the smaller of the two types of polypeptide chains present in all antibody molecules in their naturally occurring conformations. κ and λ light chains refer to the two major antibody light chain isotypes.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

“Antisense” refers particularly to the nucleic acid sequence of the non-coding strand of a double stranded DNA molecule encoding a polypeptide, or to a sequence which is substantially homologous to the non-coding strand. As defined herein, an antisense sequence is complementary to the sequence of a double stranded DNA molecule encoding a polypeptide. It is not necessary that the antisense sequence be complementary solely to the coding portion of the coding strand of the DNA molecule. The antisense sequence may be complementary to regulatory sequences specified on the coding strand of a DNA molecule encoding a polypeptide, which regulatory sequences control expression of the coding sequences.

As used herein, the term “applicator” refers to any device including, but not limited to, a hypodermic syringe, a pipette, an automatic sample probe and the like, for administering the compounds and compositions of the invention.

A “constitutive” promoter is a nucleotide sequence which, when operably linked with a polynucleotide which encodes or specifies a gene product, causes the gene product to be produced in a cell under most or all physiological conditions of the cell.

The term “container” includes any receptacle for holding a composition useful within the methods of the invention. For example, in one embodiment, the container is the packaging that contains the composition. In other embodiments, the container is not the packaging that contains the composition, i.e., the container is a receptacle, such as a box or vial that contains the packaged composition or unpackaged composition and the instructions for use of the composition. Moreover, packaging techniques are well known in the art. It should be understood that the instructions for use of the composition may be contained on the packaging containing the composition, and as such the instructions form an increased functional relationship to the packaged product. However, it should be understood that the instructions may contain information pertaining to a procedure that allows for implementation of a method of the invention.

As used herein, the term “controlled HIV infection” in a human refers to an HIV-infected human who is receiving HIV treatment and has low serum viral loads (such as, but not limited to, equal to or less than about 200 copies/mL, or equal to or less than about 400 copies/mL).

The term “derivative” includes any purposefully generated peptide that in its entirety, or in part, comprises an amino acid sequence substantially similar to a variable domain amino acid sequence of an antibody that binds one of the proteins contemplated in the invention. Derivatives of the antibodies of the present invention may be characterized by single or multiple amino acid substitutions, deletions, additions, or replacements. These derivatives may include: (a) derivatives in which one or more amino acid residues are substituted with conservative or non-conservative amino acids; (b) derivatives in which one or more amino acids are added; (c) derivatives in which one or more of the amino acids of the amino acid sequence used in the practice of the invention includes a substituent group; (d) derivatives in which amino acid sequences used in the practice of the invention or a portion thereof is fused to another peptide (e.g., serum albumin or protein transduction domain); (e) derivatives in which one or more nonstandard amino acid residues (e.g., those other than the 20 standard L-amino acids found in naturally occurring proteins) are incorporated or substituted into the amino acid sequences used in the practice of the invention; (f) derivatives in which one or more non-amino acid linking groups are incorporated into or replace a portion of the amino acids used in the practice of the invention; and (g) derivatives in which one or more amino acid is modified by glycosylation.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “endogenous” refers to any material from or produced inside an organism, cell, tissue or system.

As used herein, the term “fragment,” as applied to a protein or peptide, refers to a subsequence of a larger protein or peptide. A “fragment” of a protein or peptide may be at least about 10 amino acids in length; for example, at least about 50 amino acids in length; more preferably, at least about 100 amino acids in length; even more preferably, at least about 200 amino acids in length; particularly preferably, at least about 300 amino acids in length; and most preferably, at least about 400 amino acids in length.

The term “heterologous” as used herein is defined as DNA or RNA sequences or proteins that are derived from the different species.

The term “homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

The term “immunoglobulin” or “Ig” as used herein is defined as a class of proteins that function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

As used herein, the term “instructional material” includes a publication, a recording, a diagram, or any other medium of expression which can be used to communicate the usefulness of a compound, composition or delivery system of the invention in the kit for detecting or monitoring the conditions, diseases or disorders recited herein. Optionally, or alternately, the instructional material can describe one or more methods of detecting or monitoring the conditions, diseases or disorders in a cell or a tissue of a mammal. The instructional material of the kit of the invention can, for example, be affixed to a container that contains the identified compound, composition or delivery system of the invention or be shipped together with a container that contains the identified compound, composition or delivery system. Alternatively, the instructional material can be shipped separately from the container with the intention that the instructional material and the compound be used cooperatively by the recipient.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “isolated nucleic acid” refers to a nucleic acid segment or fragment which has been separated from sequences which flank it in a naturally occurring state, i.e., a DNA fragment that has been removed from the sequences which are normally adjacent to the fragment, i.e., the sequences adjacent to the fragment in a genome in which it naturally occurs. The term also applies to nucleic acids that have been substantially purified from other components which naturally accompany the nucleic acid, i.e., RNA or DNA or proteins, that naturally accompany it in the cell. The term therefore includes, for example, a recombinant DNA that is incorporated into a vector, into an autonomously replicating plasmid or virus, or into the genomic DNA of a prokaryote or eukaryote, or that exists as a separate molecule (i.e., as a cDNA or a genomic or cDNA fragment produced by PCR or restriction enzyme digestion) independent of other sequences. It also includes a recombinant DNA that is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

As used herein, the term “monoclonal antibody” includes antibodies that display a single binding specificity and affinity for a particular epitope. These antibodies are mammalian-derived antibodies, including murine, human and humanized antibodies.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

As used herein, the terms “patient” and “subject” and “individual” refer interchangeably to a human or a non-human mammal. Non-human mammals include, for example, livestock and pets, such as ovine, bovine, porcine, canine, feline and murine mammals. In certain embodiments, the patient or subject is human.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

By the term “specifically binds,” as used herein with respect to an antibody, is meant an antibody that recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, the term “substantially the same” amino acid sequence is defined as a sequence with at least 70%, preferably at least about 80%, more preferably at least about 90%, even more preferably at least about 95%, and most preferably at least 99% homology to another amino acid sequence, as determined by the FASTA search method in accordance with Pearson & Lipman, Proc. Natl. Inst. Acad. Sci. USA 1988, 85:2444-2448.

By the term “synthetic antibody” as used herein is meant an antibody that is generated using recombinant DNA technology, such as, for example, an antibody expressed by a bacteriophage as described herein. The term should also be construed to mean an antibody that has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

A “tissue-specific” promoter is a nucleotide sequence that, when operably linked with a polynucleotide encodes or specified by a gene, causes the gene product to be produced in a cell substantially only if the cell is a cell of the tissue type corresponding to the promoter.

The term “transfected” or “transformed” or “transduced” as used herein refers to a process by which exogenous nucleic acid is transferred or introduced into the host cell. A “transfected” or “transformed” or “transduced” cell is one that has been transfected, transformed or transduced with exogenous nucleic acid. The cell includes the primary subject cell and its progeny.

As used herein, the term “uncontrolled HIV infection” refers to an HIV-infected human who is receiving HIV treatment and yet has high serum viral loads (such as, but not limited to, equal to or greater than 1,000 copies/mL).

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter comprising an isolated nucleic acid and used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates to the unexpected discovery of a novel, non-invasive method for monitoring and/or assessing HIV viral load in a human. The method comprises analyzing an urine sample from the human for the presence of one or more protein markers that are associated with active systemic HIV replication. The method allows for the monitoring of systemic HIV replication and/or infection in a human, and/or the identification of a human with uncontrolled HIV infection.

As disclosed herein, in one aspect, a survey of the urinary proteome in subjects with highly active HIV infection was performed, and the results were then compared with published studies of the HIV-uninfected human urinary proteome. A remarkable overlap of proteins identified in the present HIV urine as compared with HIV-uninfected urine was observed: 863 of the 885 proteins found in three or more of the 19 samples of HIV urine were proteins also identified in HIV-uninfected urine. This level of correspondence indicates that the methods used herein broadly surveyed HIV urine proteomes, and that comparison with reported HIV-uninfected human urine proteomes is a valid strategy to identify candidate novel HIV urine biomarkers. HIV-1-derived proteins were not observed in urine, but several host proteins in the urine of HIV-infected subjects were not observed in multiple studies of the normal human urinary proteome. These proteins stem from a wide range of cellular processes.

In certain embodiments, the unique urine proteins found in the greatest number of samples (14 of 19) were docking protein 7 (DOK7) and dynein heavy-chain 3 (DNAH3). DOK7 is a key component for proper formation of neuromuscular synapses and has no known interaction with HIV-1. The dynein heavy-chain 2 (DNAH2) isoform was also identified as unique to HIV urine samples. The peptide identifications clearly distinguish between the two dynein heavy-chain isoforms. For example, the peptide SVLTAAGNLK identified in HIV urine samples is unique to DNAH3. Conversely, the DNAH2 peptide LLMRIGDKEVEYNTNFR, not found in isoform 3, was identified in the HIV urine samples. Thus, both of these proteins, with functionally related roles in force generation during microtubule-based movement, are independent HIV urine-specific candidate markers, despite having no known interaction with HIV-1.

This study is the first general survey of urinary proteomics in HIV-infected subjects with active systemic viral replication. While no HIV-1 specific proteins were observed, several host proteins were found exclusively in the urine of subjects infected with HIV as compared to published surveys of the non-HIV-infected human urinary proteome. These HIV specific proteomic signatures provide insights in to the human physiological response to HIV infection and potentially serve as novel HIV biomarkers in urine.

### Methods

The invention includes a method of assessing or monitoring systemic HIV viral load in an HIV-infected human patient. In certain embodiments, the patient has received or is receiving a first anti-HIV medication. In other embodiments, the patient is a new-born human. In yet other embodiments, the patient is an infant under about 18 months of age.

The method comprises obtaining a bodily sample from the human. In certain embodiments, the sample comprises urine. In other embodiments, the first anti-HIV medication comprises ART. In yet other embodiments, the patient has received or is receiving ART.

The method further comprises analyzing the test sample comprising urine from the patient for the presence and/or concentration of one or more proteins contemplated within the invention.

In certain embodiments, the test sample is processed, using methods such as but not limited to protein isolation and/or protein digestion. In other embodiments, the processed sample is analyzed by mass spectrometry, whereby the presence and/or concentration of specific peptides in the sample may be correlated with the presence and/or concentration of one or more proteins contemplated within the invention.

In certain embodiments, the sample is analyzed for the presence and/or concentration of a protein using a quantum dot assay and/or chromophore assay. Such analysis is known to those skilled in the art (Stepanenko, et al., 2011, “Modern fluorescent proteins: from chromophore formation to novel intracellular applications,” Biotechniques 51(5):313-8; Mehta, et al., “Surface modified quantum dots as fluorescent probes for biomolecule recognition,” 2014, J. Nanosci. Nanotechnol. 14(1):447-59; Geszke-Moritz & Moritz, 2013, “Quantum dots as versatile probes in medical sciences: synthesis, modification and properties,” Mater. Sci. Eng. C Mater. Biol. Appl. 33(3):1008-21).

In certain embodiments, the sample is analyzed for the presence and/or concentration of a protein contemplated within the invention using an antibody or aptamer that binds to the protein. In other embodiments, the antibody is at least one selected from the group consisting of a polyclonal antibody, monoclonal antibody, Fv, Fab, F(ab)₂, single chain antibody, human antibody, humanized antibody, and fragments and derivatives thereof. In yet other embodiments, the analysis for the presence and/or concentration of the protein contemplated within the invention comprises an immunoassay. In yet other embodiments, the immunoassay comprises at least one selected from the group consisting of immunoturbidimetry, immunonephelometry, ELISA assay, radioimmunoas say, chemiluminescence immunoassay, immunofluorescence, immunoprecipitation, immunoelectrophoresis, and flow cytometry-based immunoassay.

The method further comprises comparing the presence and/or concentration of the protein in the test data set with a control data set relating to the presence or concentration of the at least one protein in a control sample. In certain embodiments, the control sample comprises an urine sample from an untreated HIV-infected control human. In other embodiments, the untreated HIV-infected control human is the patient before receiving anti-HIV medication. In other embodiments, the control sample comprises an urine sample from an HIV-uninfected control human. In other embodiments, the control sample comprises an urine sample from an HIV-infected control human with controlled infection.

In certain embodiments, comparison of the results for the test data set and the control data set allows for the monitoring and/or assessment of the systemic HIV load in the patient.

In certain embodiments, the concentration of the protein in the patient's urine is higher by at least a multiplicity factor than the concentration of the protein in the urine sample from an HIV-uninfected control human or an HIV-infected control human with controlled infection, and the patient is identified as having uncontrolled HIV infection. In other embodiments, the concentration of the protein in the patient's urine is lower by at least a multiplicity factor than the concentration of the protein in the urine sample from an HIV-uninfected control human or an HIV-infected control human with controlled infection, and the patient is identified as having a controlled HIV infection. In other embodiments, the multiplicity factor is selected from the group consisting of about 1.1, 1.2, 1.3, 1.4, 1.5, 1.6, 1.7. 1.8, 1.9, 2, 2.25, 2.5, 2.75, 3, 3.5, 4, 5, 6, 7, 8, 9, 10, 11, 12, 13, 14, 15, 20, 30, 40, 50, 75, 100, 125, 250, 500 and 1,000.

In certain embodiments, the concentration of the protein in the patient's urine is equal to or greater than a multiplicity factor of the concentration of the protein in the urine sample from an untreated HIV-positive control human, and the patient is identified as having uncontrolled HIV infection. In other embodiments, the concentration of the protein in the patient's urine is lower than the concentration of the protein in the urine sample from an untreated HIV-positive control human, and the patient is identified as having a controlled HIV infection. In other embodiments, the multiplicity factor is selected from the group consisting of about 1, 0.95, 0.9, 0.8, 0.7, 0.6, 0.5, 0.4, 0.3, 0.2, 0.1, 0.05, 0.025, 0.01, 0.005, 0.0025, 0.001, 0.0005, 0.00025, 0.0001, 0.00005 and 0.00001.

In certain embodiments, the patient is identified as having controlled HIV infection, and the patient continues to be prescribed the first anti-HIV medication.

In certain embodiments, the patient is identified as having an uncontrolled HIV infection, and the patient is prescribed a second anti-HIV medication.

In certain embodiments, the patient is identified as having an uncontrolled HIV infection and has not received any anti-HIV medication (such as for example a new-born), and the patient is prescribed an anti-HIV medication.

In certain embodiments, the at least one protein has an accession number selected from the group consisting of Q8TD57 (SEQ ID NO:1), Q18PE1 (SEQ ID NO:2), Q8NFH5 (SEQ ID NO:3), Q8WYL5 (SEQ ID NO:4), Q8IYD8 (SEQ ID NO:5), O14654 (SEQ ID NO:6), Q96AP4 (SEQ ID NO:7), Q9UQ35 (SEQ ID NO:8), Q8N6W0 (SEQ ID NO:9), Q9H792 (SEQ ID NO:10), Q9H497 (SEQ ID NO:11), Q9UE35 (SEQ ID NO:12), 000743 (SEQ ID NO:13), Q8WXF8 (SEQ ID NO:14), P81274 (SEQ ID NO:15), Q8NG08 (SEQ ID NO:16), Q96AE7 (SEQ ID NO:17), Q9BZM4 (SEQ ID NO:18), Q5T2D3 (SEQ ID NO:19), Q8IXT5 (SEQ ID NO:20), Q9P225 (SEQ ID NO:21), and Q9Y2I9 (SEQ ID NO:22).

In certain embodiments, the at least one protein has an accession number selected from the group consisting of P41222 (PTGDS) (SEQ ID NO:23), P14151 (SELL) (SEQ ID NO:24), Q06418 (TYRO3) (SEQ ID NO:25), P52306 (RAP1GDS1) (SEQ ID NO:26), and Q9Y5Y7 (LYVE1) (SEQ ID NO:27).

### Antibodies

Using conventional techniques, the skilled artisan may use the nucleotide and amino acid sequences of the proteins contemplated within the invention to prepare an antigenic peptide for use in generating corresponding antibody. The sequence for the proteins contemplated within the invention are listed in Tables 1-2.

Alternatively, the skilled artisan may utilize a commercially available antibody against a protein contemplated within the invention. The skilled artisan may also obtain commercially available antibodies and modify them using conventional methods such as coupling to other antibodies, partial digestion, pegylation or covalent modification. Modified antibodies may then be used in the methods of the invention as described herein. Antibodies useful in the practice of the present invention may be polyclonal, monoclonal, synthetic or fragments of any of the above.

It will be appreciated that an antibody used in the invention may be monovalent, divalent or polyvalent in order to achieve antigen binding. Monovalent immunoglobulins are dimers (HL) formed of a hybrid heavy chain associated through disulfide bridges with a hybrid light chain. Divalent immunoglobulins are tetramers (H2L2) formed of two dimers associated through at least one disulfide bridge.

The invention also includes functional equivalents of the antibodies described herein. Functional equivalents have binding characteristics comparable to those of the antibodies, and include, for example, hybrid and single chain antibodies, as well as fragments thereof. Methods of producing such functional equivalents are disclosed for example in PCT Application Nos. WO 1993/21319 and WO 1989/09622. Functional equivalents include polypeptides with amino acid sequences substantially the same as the amino acid sequence of the variable or hypervariable regions of the antibodies raised against proteins contemplated within the invention, according to the practice of the present invention.

Functional equivalents of the antibodies further include fragments of antibodies that have the same, or substantially the same, binding characteristics to those of the whole antibody. Such fragments may contain one or both Fab fragments or the F(ab′)2 fragment. Preferably the antibody fragments contain all six complement determining regions of the whole antibody, although fragments containing fewer than all of such regions, such as three, four or five complement determining regions, are also functional. The functional equivalents are members of the IgG immunoglobulin class and subclasses thereof, but may be or may combine any one of the following immunoglobulin classes: IgM, IgA, IgD, or IgE, and subclasses thereof. Heavy chains of various subclasses, such as the IgG subclasses, are responsible for different effector functions and thus, by choosing the desired heavy chain constant region, hybrid antibodies with desired effector function are produced. Preferred constant regions are gamma 1 (IgG1), gamma 2 (IgG2 and IgG), gamma 3 (IgG3) and gamma 4 (IgG4). The light chain constant region can be of the kappa or lambda type.

The monoclonal antibodies may be advantageously cleaved by proteolytic enzymes to generate fragments retaining the antigen binding site. For example, proteolytic treatment of IgG antibodies with papain at neutral pH generates two identical so-called “Fab” fragments, each containing one intact light chain disulfide-bonded to a fragment of the heavy chain (Fc). Each Fab fragment contains one antigen-combining site. The remaining portion of the IgG molecule is a dimer known as “Fc”. Similarly, pepsin cleavage at pH 4 results in the so-called F(ab′)2 fragment.

Single chain antibodies or Fv fragments are polypeptides that consist of the variable region of the heavy chain of the antibody linked to the variable region of the light chain, with or without an interconnecting linker. Thus, the Fv comprises an antibody combining site.

Hybrid antibodies may be employed. Hybrid antibodies have constant regions derived substantially or exclusively from human antibody constant regions and variable regions derived substantially or exclusively from the sequence of the variable region of a monoclonal antibody from each stable hybridoma.

Methods for preparation of fragments of antibodies are known to those skilled in the art. See, Goding, “Monoclonal Antibodies Principles and Practice”, Academic Press (1983), p. 119-123. Fragments of the monoclonal antibodies containing the antigen binding site, such as Fab and F(ab′)2 fragments, may be preferred in therapeutic applications, owing to their reduced immunogenicity. Such fragments are less immunogenic than the intact antibody, which contains the immunogenic Fc portion. Hence, as used herein, the term “antibody” includes intact antibody molecules and fragments thereof that retain antigen binding ability.

When the antibody used in the practice of the invention is a polyclonal antibody (IgG), the antibody is generated by inoculating a suitable animal with a protein contemplated within the invention, or a fragment thereof. Antibodies produced in the inoculated animal that specifically bind to a protein contemplated within the invention are then isolated from fluid obtained from the animal. Antibodies may be generated in this manner in several non-human mammals such as, but not limited to, goat, sheep, horse, rabbit, and donkey. Methods for generating polyclonal antibodies are well known in the art and are described, for example in Harlow et al. (In: Antibodies, A Laboratory Manual, 1988, Cold Spring Harbor, N.Y.). These methods are not repeated herein as they are commonly used in the art of antibody technology.

When the antibody used in the methods used in the practice of the invention is a monoclonal antibody, the antibody is generated using any well-known monoclonal antibody preparation procedures such as those described, for example, in Harlow et al. (In: Antibodies, A Laboratory Manual, 1988, Cold Spring Harbor, N.Y.) and Tuszynski et al. (Blood 1988, 72: 109-115). Given that these methods are well known in the art, they are not replicated herein. Generally, monoclonal antibodies directed against a desired antigen are generated from mice immunized with the antigen using standard procedures as referenced herein. Monoclonal antibodies directed against full length or fragments of target structure may be prepared using the techniques described in Harlow et al. (In: Antibodies, A Laboratory Manual, 1988, Cold Spring Harbor, N.Y.).

The skilled artisan would further appreciate, based upon the disclosure provided herein, that the invention is not limited to the use of an antibody as the binding element for a protein contemplated within the invention. The invention also allows for the use of an non-antibody molecule as the element that binds to one or more of the proteins that are contemplated in the invention. The non-antibody molecule may bind to the protein or a fragment of the protein. Preferred non-antibody molecules within the invention are aptamers. Aptamers are oligonucleic acid (also referred to as nucleic acid) molecules or peptide molecules that bind a specific target molecule. Nucleic acid aptamers are nucleic acid species that have been engineered through repeated rounds of in vitro selection or equivalently, SELEX (systematic evolution of ligands by exponential enrichment), to bind to various molecular targets such as small molecules, proteins, nucleic acids, and even cells, tissues and organisms. Aptamers are useful in biotechnological and therapeutic applications as they offer molecular recognition properties that rival that of the commonly used antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. See Ellington & Szostak, 1990, Nature 346(6287):818-22; Bock, et al., 1992, Nature 355(6360):564-6; Drabovich, et al., 2006, Anal. Chem. 78(9):3171-8, all of which are incorporated herein by reference in their entireties. Aptamers useful within the invention may be selected and/or prepared according to the teachings of the art.

The binding of the antibody to the protein contemplated within the invention may be analyzed using any appropriate immunoassay available and/or known to those skilled in the art. Immunoassays are based on specific binding of an antibody to its antigen (in this particular case, the protein contemplated within the invention). Detecting the interaction of the antibody with the antigen may be achieved using a variety of methods, of which one of the most common is to label either the antigen or antibody, and monitor the change in environment of the label upon binding. The label may comprise an enzyme (wherein binding is monitored by enzyme immunoassay or EIA), colloidal gold (wherein binding is monitored by lateral flow assays), radioisotopes such as ¹²⁵I radioimmunoassay (wherein binding is monitored by radiometric methods), magnetic labels (wherein binding is monitored by magnetic immunoassay or MIA) or fluorescence. Other techniques include, but are not limited to, agglutination, nephelometry, turbidimetry and Western Blot. All of these methods are known to those of skill in the art. See e.g. Harlow, et al., 1988, “Antibodies: A Laboratory Manual”, Cold Spring Harbor Laboratory Press, Cold Spring Harbor, N.Y.; Harlow, et al., 1999, “Using Antibodies: A Laboratory Manual, Cold Spring Harbor Laboratory Press”, Cold Spring Harbor, N.Y.

Immunoassays may be divided into those that involve non-labelled reagents and those that involve labelled reagents. Immunoassays that involve labelled reagents are divided into homogenous immunoassays and heterogeneous immunoassays (the latter require an extra step to remove unbound antibody or antigen from the site, usually using a solid phase reagent).

Heterogeneous immunoassays may be competitive or non-competitive. In a competitive immunoassay, the antigen in the unknown sample competes with labeled antigen to bind with antibodies. The amount of labeled antigen bound to the antibody site is then measured. In this method, the response will be inversely proportional to the concentration of antigen in the unknown, since a large response indicates that there is little antigen in the unknown to compete with the labeled antigen. In noncompetitive immunoassays, also referred to as the “sandwich assay,” antigen in the unknown is bound to the antibody site, then labeled antibody is bound to the antigen. The amount of labeled antibody on the site is then measured. Unlike the competitive method, the results of the noncompetitive method are directly proportional to the concentration of the antigen, since the labeled antibody will not bind if the antigen is not present in the unknown sample.

In certain embodiments, the immunoassay is selected from the group consisting of immunoturbidimetry, immunonephelometry, an ELISA assay, radioimmunoas say, chemiluminescence immunoassay, immunofluorescence, immunoprecipitation, immunoelectrophoresis, and flow cytometry-based immunoassay.

One skilled in the art will recognize that optimization studies may be easily performed to determine which chemical reagent(s) present in solution do, or do not, significantly interfere with the selective binding of the antibody to the antibody. The optimization studies may involve the use of two samples, one comprising the protein of interest and the chemical reagent, and the second comprising the protein of interest but devoid of the chemical reagent. The two samples are separately incubated with the antibody. Non-limiting examples of such chemical reagents are surfactants, non-ionic surfactants, divalent cation salts, dextran salts, PEG, α-cyclodextrin salts, EDTA, and azide salts. Following incubations, an immunoassay is used to determine the degree of antibody binding for each sample, and this information is used to determine the effect of the chemical reagent on the antibody-antigen binding. This evaluation follows standard methodologies used in analytical sciences and should not require unwarranted experimentation from those skilled in the art.

The immunoassay used to detect the interaction of the antibody with the protein of interest may also be used to quantitate the concentration of the protein in the sample. In a typical procedure included in the invention, a series of standard solutions containing known concentrations of the protein of interest are prepared and analyzed by an immunoassay. The readings obtained for each standard solution are used to create a calibration curve. The unknown sample is then analyzed by the same immunoassay and its reading is compared to the standard curve in order to obtain a corresponding concentration of the protein of interest in the sample. This concentration may be used to calculate the actual concentration of the protein of interest in the biological fluid, taking into account the dilutions that the biological sample was subjected to for the preparation of the test sample.

Use of the calibration curve, as described above, allows the concentration of the protein to be determined in the same units used to express the concentration of the standard solutions. In some instances, the standard solutions have their component concentrations identified in mass/volume units (such as mg/dL units, for example). The concentration of the protein of interest in the biological sample, determined as mg/dL from the calibration curve, may be converted to a concentration of moles/volume (such as nmol/L) based on the molecular weight of the protein of interest.

As will be understood by one of skill in the art, when armed with the disclosure set forth herein, a set of reference proteins or equivalents (also referred to as “calibration samples”) may be used to create a calibration curve for a certain method and/or instrument. By way of a non-limiting example, the set of reference proteins or equivalents may be used in a one- or two-point calibration assay. In another embodiment of the invention, the set of reference proteins or equivalents may be used in a three-, four-, five- or six point calibration assay. In one aspect, the set of reference proteins or equivalents may include as many or as few reference points as determined to be necessary to establish a valid and accurate reference curve.

Numerous calibration schemes may be used in the clinical laboratory. Some methods, often manually performed, employ several concentration levels throughout the assay range and typically plot the instrumental response versus concentration or use linear regression to calculate patient analyte values. With the increasing use and availability of computer technology, methods often use one or two calibrator points to achieve the same results. Quite often, the one or two set point method incorporates a saline or distilled water blank as an additional set point, this latter function being dictated by the instrument or reagent manufacturer. For non-linear chemistries, the traditional approach provides five or six levels of calibrator, usually set in a non-linear fashion dictated by the mathematical model used in the final calculation of patient result. A more recent trend for non-linear chemistries is to use one calibrator containing the highest concentration of analyte measured in the assay. Using this method, the analytical system is then directed to perform the necessary dilutions of this high concentration value to generate the predetermined calibration set points on the fly when the system calibrates the analyte. A four- or five-parameter logit/log calibration curve is typically used for automated immunoassays.

Therefore, in an aspect of the present invention, there is provided a method that features the use of multiple calibrator points in order to generate a reference curve. In one embodiment, the method features the use of more than one point. In another embodiment, one of the multiple points is a zero point. In yet another embodiment, the zero point is not included as one of the multiple points, but may be included separately in a reference curve. In another embodiment, the method features the use of a single calibration point, as described in detail elsewhere herein. In yet another embodiment, the method features the use of a zero point in addition to a single calibration point.

By way of a series of non-limiting examples, the method of the invention may use a reference curve based on a single concentration for calibration, a reference curve based on a single concentration plus a zero concentration point for calibration, a reference curve based on at least two concentrations for calibration, or a reference curve based on at least two concentrations plus a zero concentration point for calibration. In one embodiment of the invention, the concentration of a calibration sample is known. In yet another embodiment of the invention, the concentration of at least one calibration sample in a mixture containing at least two calibration samples is known.

### Kits

The invention includes various kits that comprise a set of protein antibodies, or equivalents thereof, an applicator, and instructional materials that describe the use of the kit to perform the methods of the invention. Although exemplary kits are described below, the contents of other useful kits will be apparent to the skilled artisan in light of the present disclosure. Each of these kits is included within the invention. The kit is used pursuant to the methods disclosed in the invention.

In certain embodiments, the invention includes a kit for measuring the concentration of at least one protein contemplated in the invention in a biological sample of a patient. In other embodiments, the biological sample comprises urine. The kit may comprise reagents, such as antibodies or equivalents thereof, that allow for the determination of the at least one protein contemplated in the invention. The kit further comprises an applicator and instructional material for the use of the kit.

The kit may further comprise an applicator useful for administering the reagents for use in the relevant assay. The particular applicator included in the kit will depend on, e.g., the method used to assay the protein, as well as the particular analyzer equipment used, and such applicators are well-known in the art and may include, among other things, a pipette, a syringe, a dropper bottle, and the like. Moreover, the kit may comprise an instructional material for the use of the kit.

Further, the invention includes a kit comprising at least one reference composition comprising a known value of a known constituent, which may be a protein, a derivative thereof or a fragment thereof. Such kits may be used to create a calibration curve for quantitation of the protein. Thus, the invention encompasses a kit comprising at least one reference composition. While the invention is not limited to any particular set, certain combinations of reference compositions are exemplified elsewhere herein.

In certain embodiments, the invention includes a kit for assessing or monitoring systemic HIV viral load in an HIV-infected human patient, the kit comprising an antibody or aptamer that binds to at least one protein contemplated within the invention; an applicator; and, an instructional material for the use of the kit, wherein the instruction material comprises instructions for analyzing a test sample comprising urine from the patient for the presence or concentration of the at least one protein.

In certain embodiments, the kit further comprises a test data set with a control data set relating to the presence or concentration of the at least one protein in a control sample. In other embodiments, the control sample comprises an urine sample from an untreated HIV-infected control human and/or an HIV-negative control human and/or an HIV-infected control human with controlled infection.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Methods and Materials

### Sample Collection and Processing

Subjects were asked to refrain from consuming alcohol and nonprescription drugs for 24 hours prior to sample collection but were allowed to maintain a normal diet otherwise. Subjects provided their second void of the day after approximately 5 mL of urine had been passed. Samples were promptly placed on ice, centrifuged at 2000×g for 20 minutes at 4° C. to remove any cells that may have been extraneously passed, and stored at −70° C.

### Protein Isolation and Digestion

Urine solutions were brought to 8 M urea, 10 mM dithiothreitol, 100 mM Tris HCl, pH 7.6, and concentrated using a 30-kD Amicon molecular-weight cutoff (MWCO) device (Millipore, Billerica, Mass.). Concentrated proteins were depleted of albumin using a Cibracron blue-based method (Pierce, Rockford, Ill.). Immunoglobulins were depleted using the “top 2” abundant-protein depletion column from Thermo Pierce (http://www dot piercenet dot com/product/abundant-protein-depletion-spin-columns).

A volume of urine containing 500 μg of total protein was buffer exchanged to 10 mM PBS and 0.15 M NaCl using a 3-kD MWCO spin filter (Millipore) and loaded to the depletion column. The sample was incubated in the column for 30 minutes, reverse transcribed, and mixed at 500 rpm (MixMate, Eppendorf, Hamburg, Germany). Following incubation the column was spun and the depleted sample collected for further processing. Depleted protein samples were transferred to a 30-kD Amicon MWCO device (Millipore) and centrifuged at 3,000×g for 30 minutes. The remaining sample was buffer exchanged with 6 M urea, 100 mM Tris HCl, pH 7.6, then alkylated with 55 mM iodoacetamide. Concentrations were measured using a Qubit fluorometer (Invitrogen, Carlsbad, Calif.). Trypsin was added at a ratio of 1:40 enzyme to substrate and the sample incubated overnight on a heat block at 37° C. The device was centrifuged at 3,000×g for 30 minutes and the filtrate collected.

### Peptide Desalting

Digested peptides were desalted using C18 stop-and-go extraction (STAGE) tips. For each sample, a C18 STAGE tip was activated with methanol, then conditioned with 60% acetonitrile/0.5% acetic acid, followed by 5% acetonitrile/0.5% acetic acid. Samples were loaded onto the tips and desalted with 0.5% acetic acid. Peptides were eluted with 60% acetonitrile/0.5% acetic acid and lyophilized in a SpeedVac (Thermo Savant) to dryness, for approximately 2 h.

### Liquid Chromatography-Tandem Mass Spectrometry

Each fraction was analyzed by reverse-phase liquid chromatography coupled to tandem mass spectrometry (LC-MS/MS). LC was performed on a Thermo Easy NanoLC II system. Mobile phase A included 94.5% Milli-Q water (Millipore) and 5% acetonitrile/0.5% acetic acid. Mobile phase B included 80% acetonitrile, 19.5% Milli-Q water, and 0.5% acetic acid. The 120-minute LC gradient ran from 0% B to 35% B over 90 minutes, with the remaining time used for sample loading and column regeneration. Samples were loaded to a 2 cm×100-μm inside-diameter trap column. The analytical column was 13 cm×75 μm inside-diameter fused silica with a pulled tip emitter. Both trap and analytical columns were packed with 3.5-μm C18 resin (Magic C18AQ, Michrom, Fremont, Calif.). The LC was interfaced to a dual-pressure linear ion trap mass spectrometer (LTQ Velos, Thermo Fisher) via nanoelectrospray ionization. An electrospray voltage of 1.8 kV was applied to a precolumn tee. The mass spectrometer was programmed to acquire, by data-dependent acquisition, tandem mass spectra from the top 15 ions in the full scan from 400 to 1400 m/z.

### Data Processing and Library Searching

Mass spectrometer RAW data files were converted to Mascot generic format (MGF) using msconvert. All searches required strict tryptic cleavage, 0 or 1 missed cleavages, fixed modification of cysteine alkylation, variable modification of methionine oxidation, and expectation value scores of 0.01 or lower. MGF files were searched using X!Hunter against the latest spectral library available in the Global Proteome Machine database at the time. X!!Tandem and OMSSA (Open Mass Spectrometry Search Algorithm) searches used Ensembl protein sequence libraries. The human sequence library used in this analysis was the Ensembl Genome Browser (“Human”) (http://useast dot ensembl dot org/Homo_sapiens/Info/Index). MGF files were searched using X!!Tandem using both the native and k-score8 scoring algorithms and OMSSA. All searches were performed on Amazon (Seattle, Wash.) Web Services-based Cluster Compute instances using the Proteome Cluster interface. XML output files were parsed and nonredundant protein sets were determined using in-house scripts. Proteins were required to have 1 or more unique peptides with peptide E-value scores of ≦0.01 from X!!Tandem, ≦0.01 from OMSSA, ≦0.001 and theta values of ≧0.5 from X!Hunter searches, and protein E-value scores of ≦0.0001 from X!!Tandem and X!Hunter.

Proteins identified in ≧3 HIV-infected urine samples were then compared with published studies of the human urinary proteome to assess potential uniqueness to the urinary proteome of the HIV-infected. Unique urine proteins in the HIV-infected were searched for in the HIV-1, Human Protein Interaction Database and Host Proteins in HIV-1 database in order to report known relevance in HIV biology. Gene ontology information was derived from www dot uniprot dot org.

### Example 1

### Study Population

Subjects from the Drexel University College of Medicine HIV clinic were enrolled in this single-center study. Eligible patients included those aged ≧18 years with clade B chronic HIV-1 infection free of baseline resistance based on genotype or phenotype testing, with fewer than 2 weeks of intervening antiretroviral therapy, and an HIV-1 serum viral load ≧50,000 copies/mL in the prior 30 days.

Exclusion criteria were:

chronic hepatitis B virus (HBV) or hepatitis C virus (HCV) infection as defined by positive results from serology for HBV surface antigen or detectable HCV viral load by polymerase chain reaction, respectively;

evidence of active infection in the prior 2 weeks;

treatment for acute opportunistic infection, including Pneumocystis jiroveci pneumonia, Toxoplasma gondii encephalitis, cryptosporidiosis, microsporidiosis, Mycobacterium tuberculosis disease, disseminated Mycobacterium avium complex disease, bacterial pneumonia, bacterial enteric disease, bartonellosis, syphilis, mucocutaneous candidiasis, cryptococcosis, histoplasmosis, coccidioidomycosis, aspergillosis, cytomegalovirus disease, herpes simplex virus disease, varicella zoster virus disease, human herpesvirus-8 disease, or progressive multifocal leukoencephalopathy caused by JC virus;

hematuria on screening urinalysis in the past 30 days;

chemotherapy, radiotherapy, or immunotherapy in the past 30 days except for topical or inhaled steroids;

positive nucleic acid amplification testing of genitourinary tract for Neisseria gonorrhoeae or Chlamydia trachomatis in the prior 2 weeks; or

any other medical condition that rendered the subject unable to complete the study, interfered with participation, or produced significant risk to the subject.

### Example

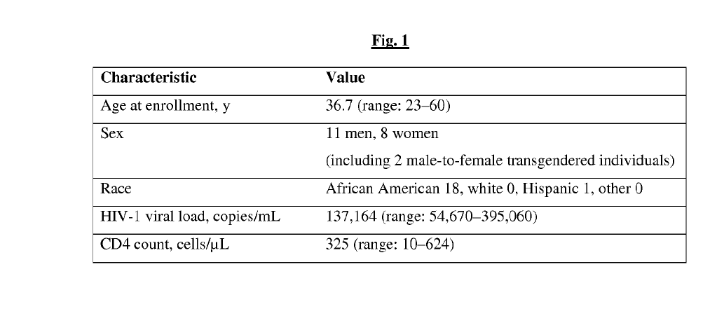

Urine samples from 19 subjects with clade B chronic HIV-1 infection having serum viral loads ≧50,000 copies/mL in the prior 30 days were collected and frozen for subsequent analysis (characteristics of study population are illustrated in FIG. 1). Albumin is generally the major protein constituent of urine and thus may prevent proteomic identification of lower-abundance HIV proteins or unique host biomarkers of HIV infection. Thus, urine samples were depleted of albumin.

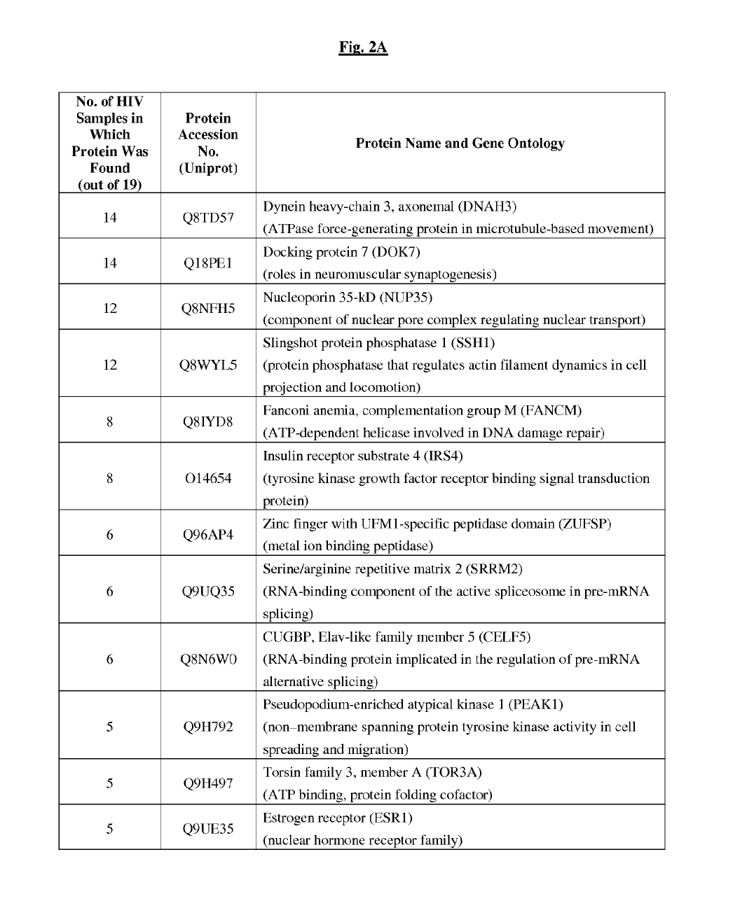

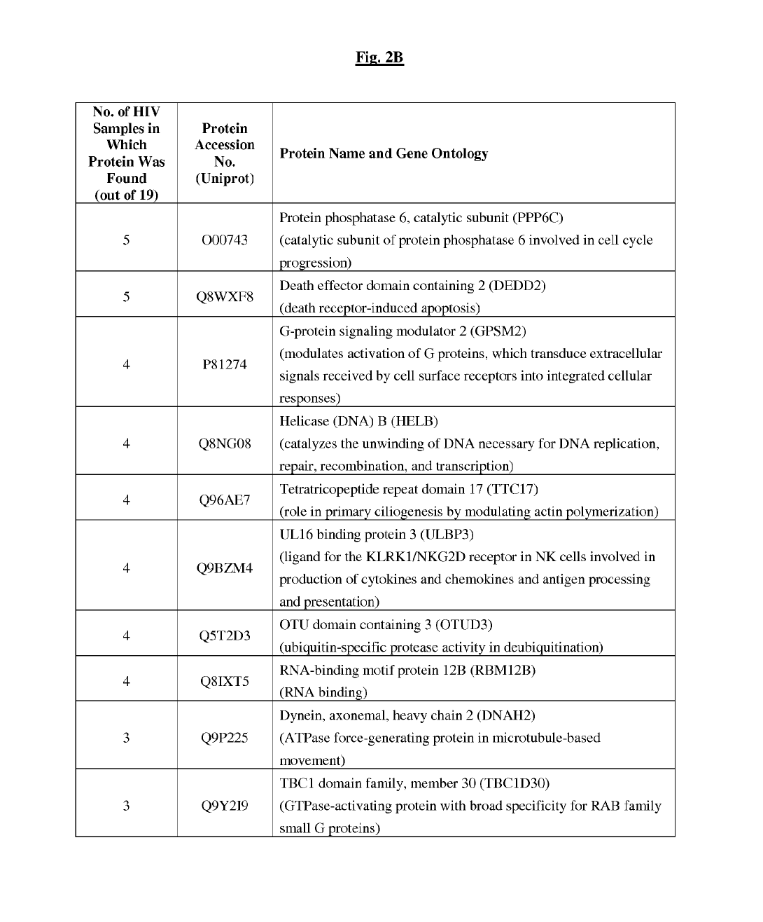

HIV infection is associated with a chronic inflammatory state, and thus anticipating high levels of immunoglobulin in the urine (which might also hinder identification of potential lower-abundance HIV peptides or host biomarkers), IgG was depleted from the urine samples. Raw data queried against HIV sequence databases did not identify any HIV-specific peptides. In searches against the human Fasta sequence database, combined analysis of all 19 samples (two of which were analyzed twice using the same LC-MS/MS method) identified a total of 37,886 peptides corresponding to 1794 human-unique proteins. Compared to studies that have sought to comprehensively characterize the human urinary proteome, 22 proteins unique to HIV-infected urine were identified (FIG. 2).

### Example 2

The subjects had a mean age of 41 years. The subjects were 60% male, 32% female, and 8% transgender; were 88% Black, 8% Hispanic, and 4% White; had a median serum HIV viral load of 108,960 copies/mL; and a median CD4 count of 340 cells/μL.

Urine samples were collected from 20 adults with wild type clade B HIV-1 infection and an HIV-1 serum viral load ≧50,000 copies/mL within 30 days.

Subjects were free of Neisseria gonorrhoeae or Chlamydia trachomatis urethritis, active or opportunistic infection, and hematuria. Samples were centrifuged to remove cellular debris and then frozen to −70° C. Thawed samples were concentrated then depleted of albumin ±immunoglobulins.

100 μg of each sample were lyophilized and suspended in denaturing buffer before reduction, alkylation, and enzymatic digestion with sequencing grade trypsin. Samples underwent strong cation exchange before liquid chromatography coupled to tandem mass spectrometry (MS) with CID fragmentation. Datasets were searched against HIV and fasta human protein databases with Bioworks Sequest algorithm and Protein Prospector. Sequest X-correct scores of 2.5 for doubly charged and 3 for triply charged, and Protein Prospector scores of 20 were used as initial thresholds for peptide identification. Spectral counts corresponding to peptide identifications were used to reflect relative abundance. Unique HIV urine peptide and protein signatures were identified through comparison with reported urine proteomes from non-HIV infected persons.

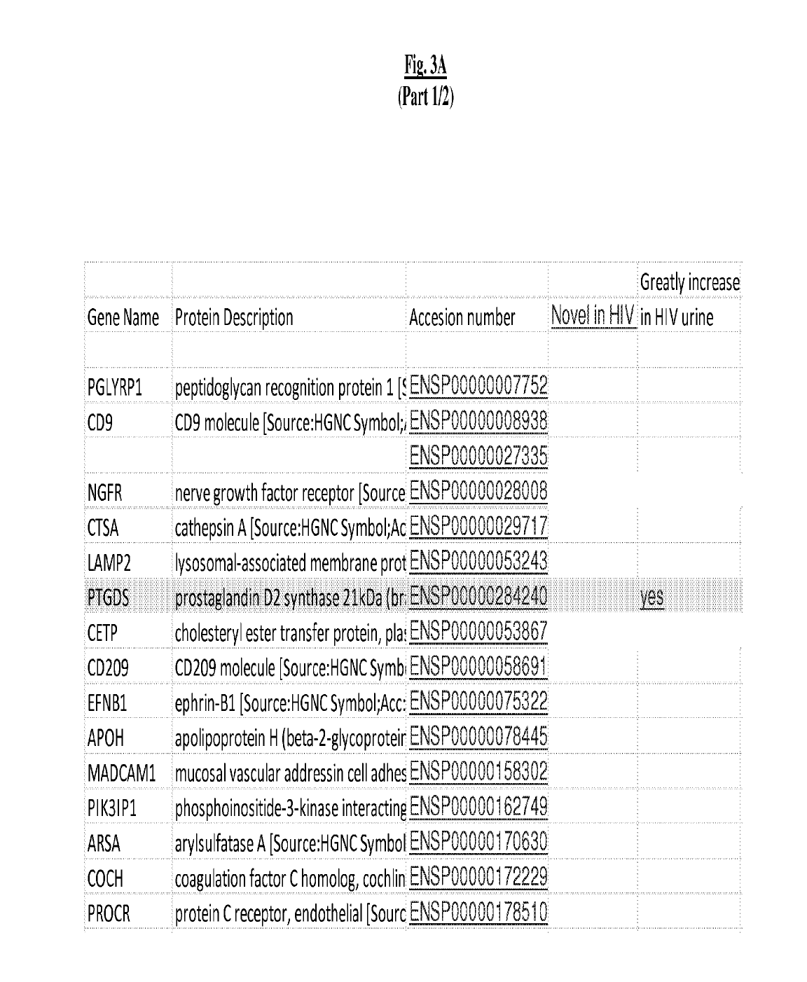

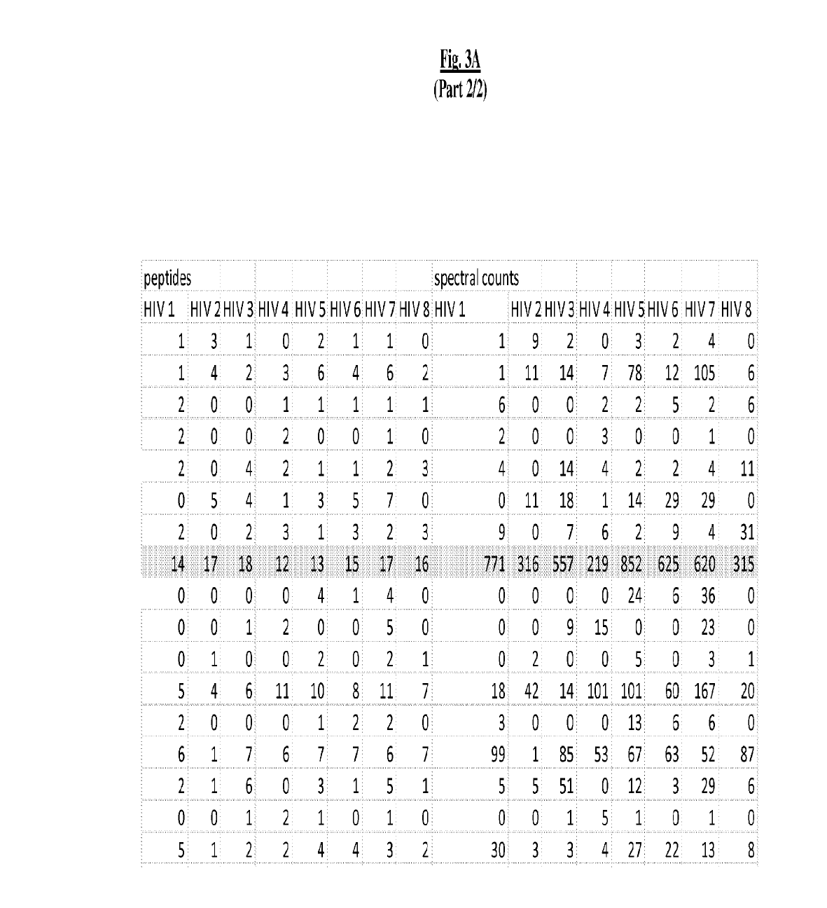

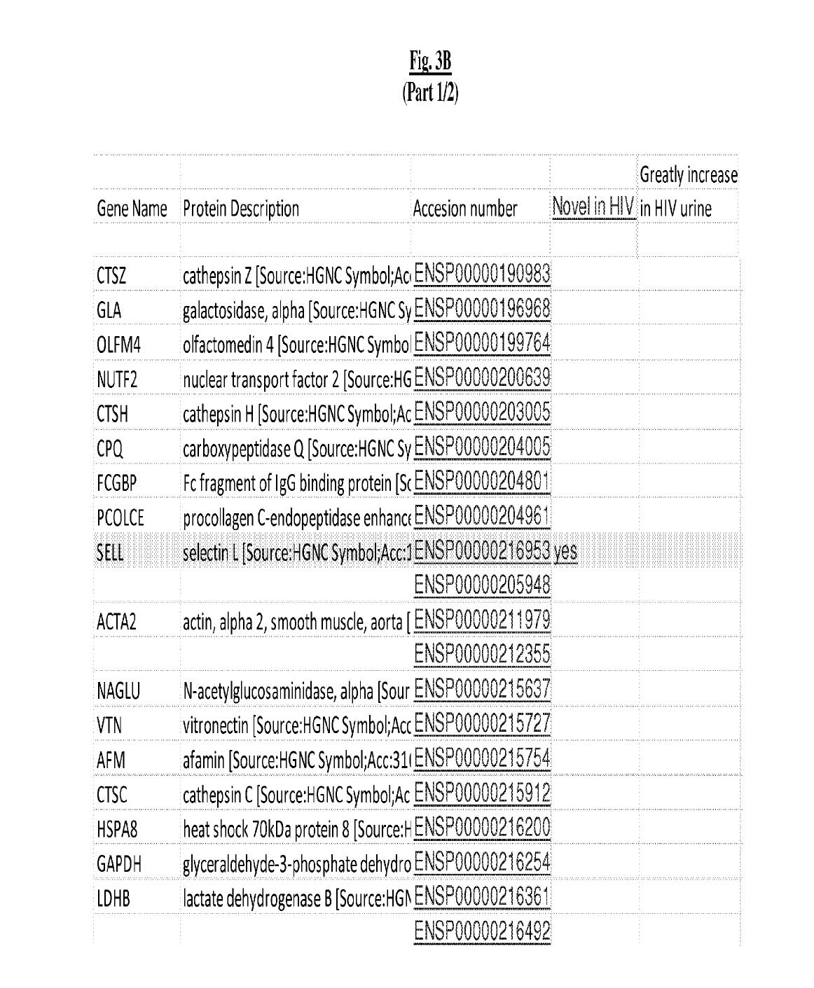

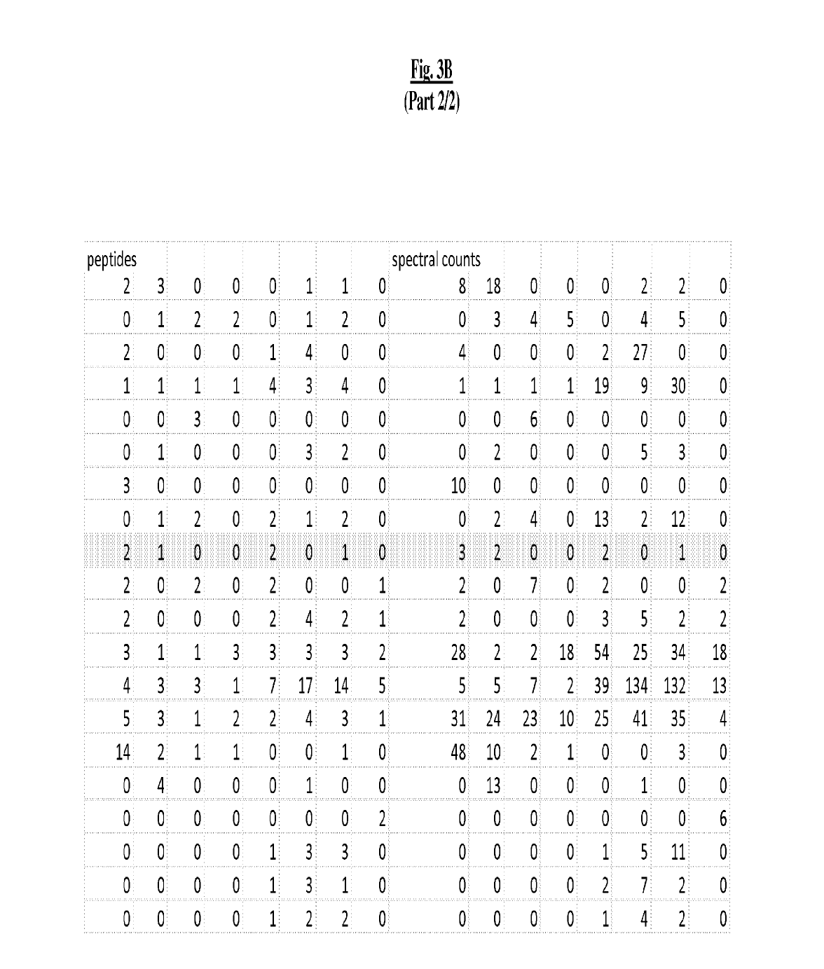

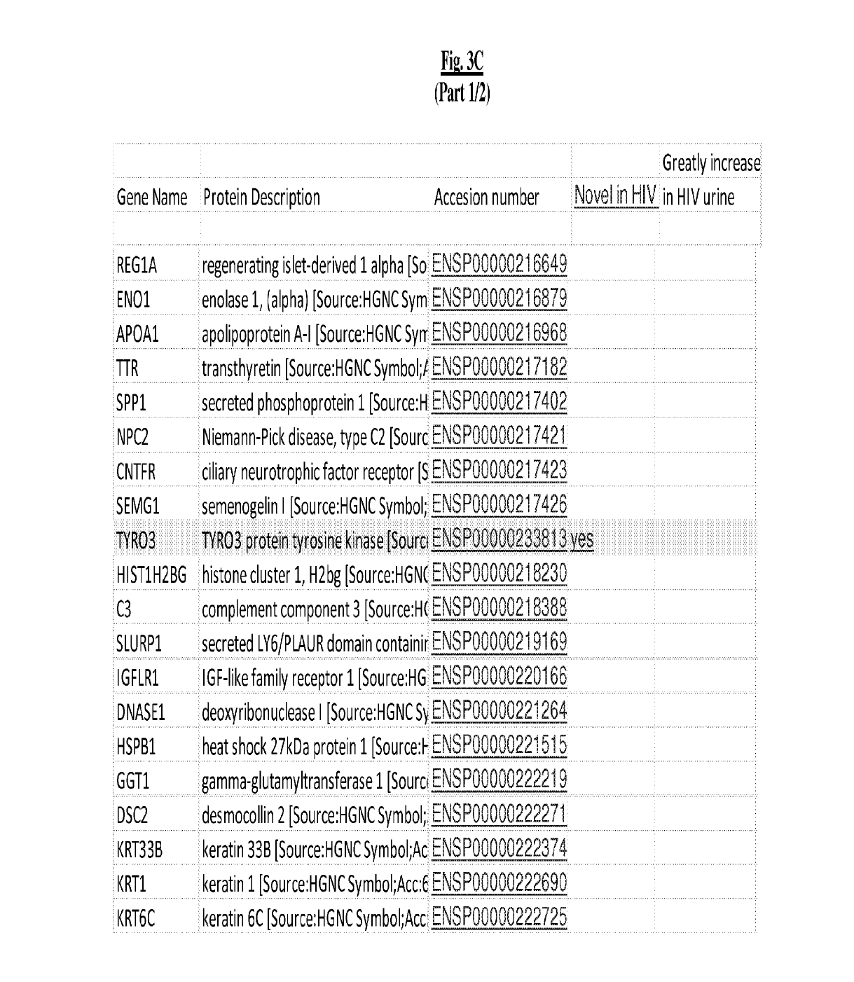

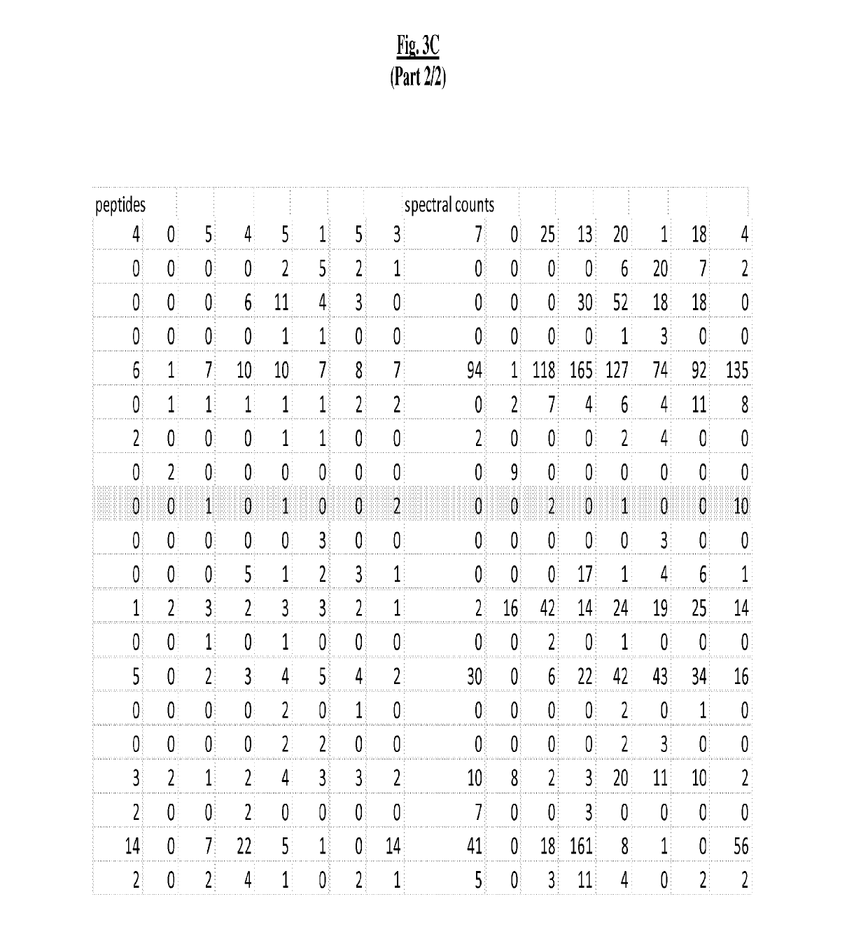

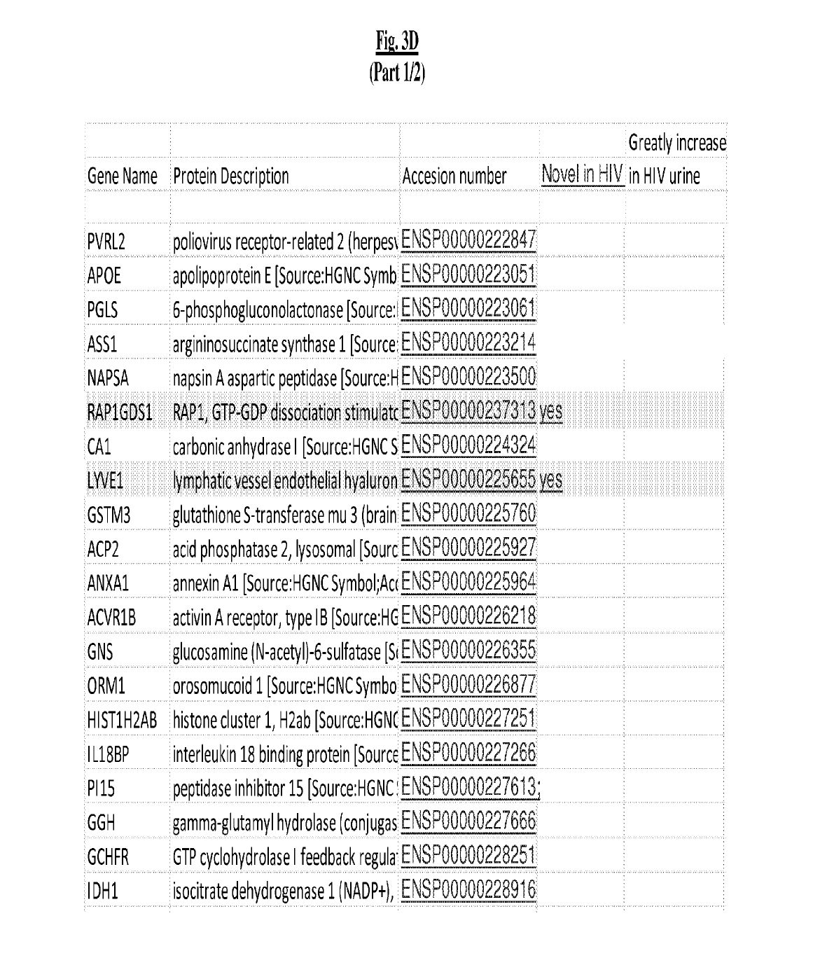

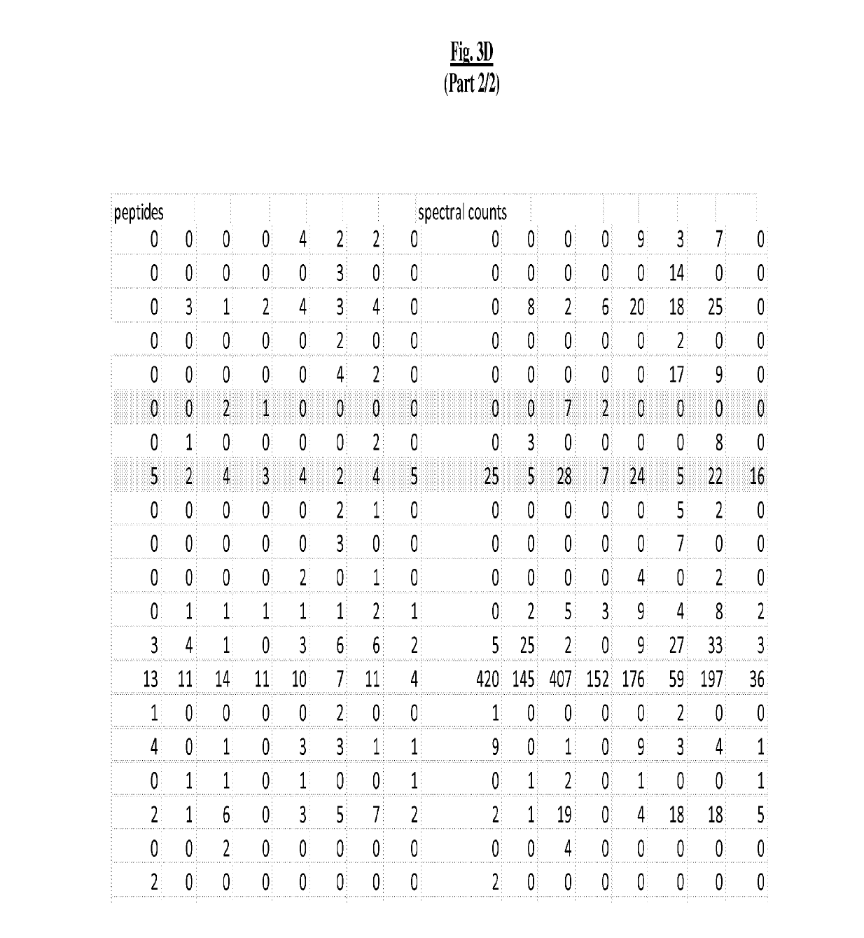

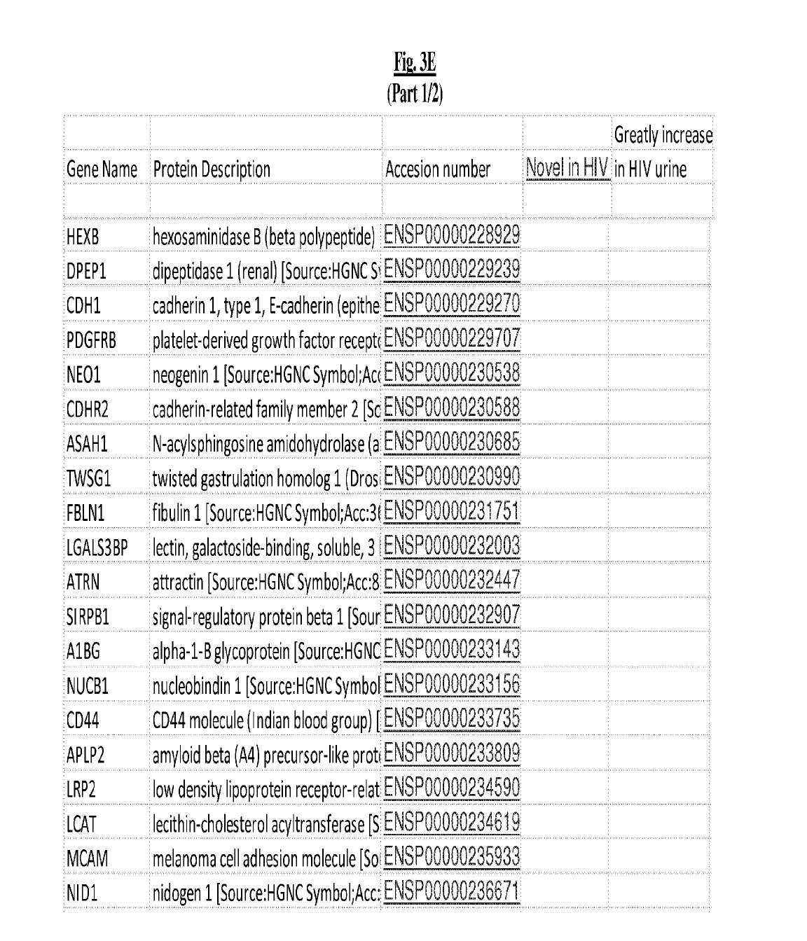

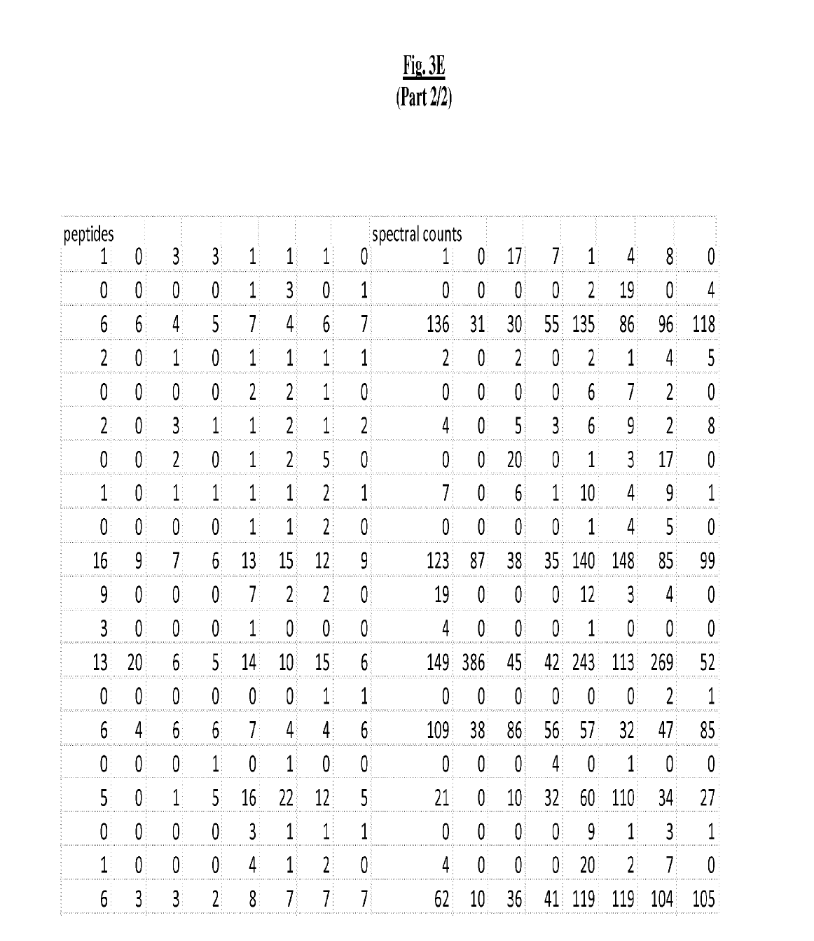

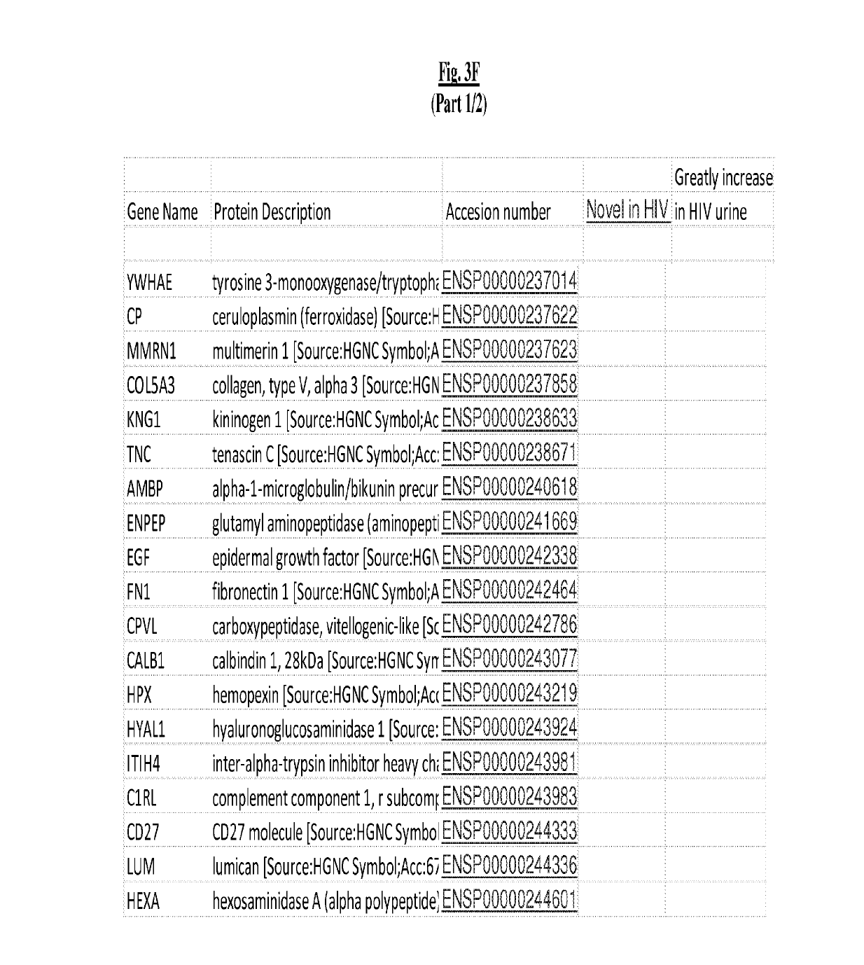

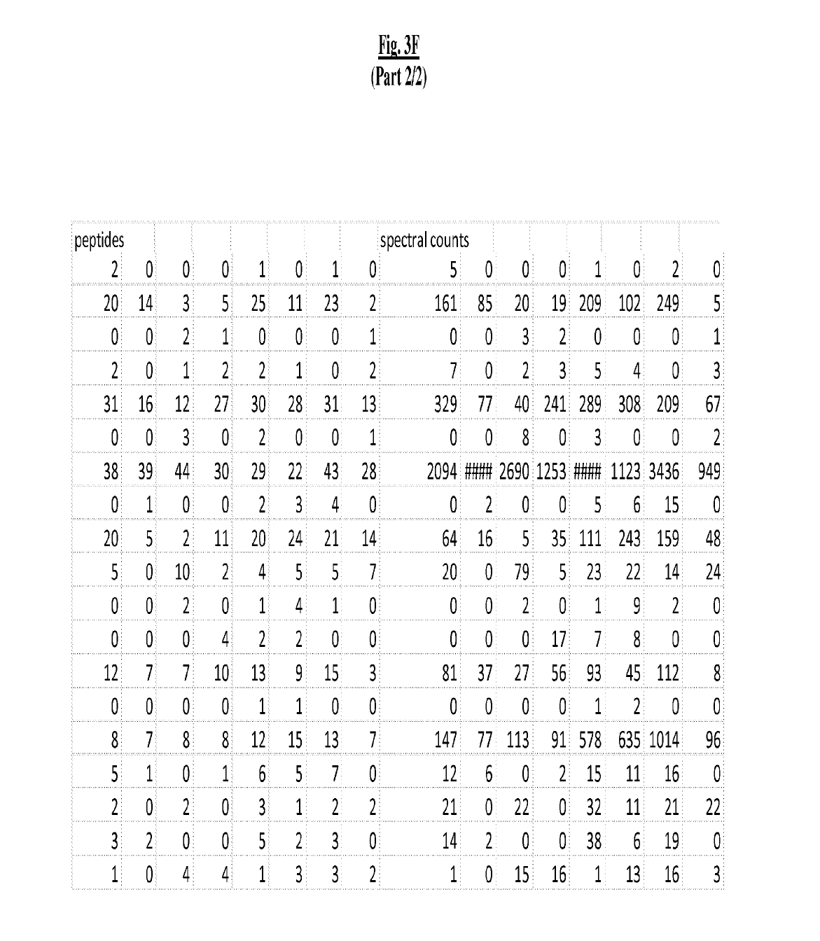

About 1,500 peptides of about 400 unique proteins were identified in the urine samples (FIG. 3). HIV-derived peptides were not observed. In all cases, a non-immunoglobulin specific protein identified in more than two of the HIV urine samples was also found in reported non-HIV urine proteomes. Several urine markers appeared to be significantly more abundant in HIV urine, including prostaglandin D2, which was found in every HIV urine sample and represented about the 6^{th} most abundant protein (as compared to about the 100^{th} most abundant protein in non-HIV urine samples). Other markers were unique to only the HIV-urine proteomes, such as L-selectin (10 of 20 samples) and lymphatic vessel endothelial hyaluronan receptor 1 (20 of 20 samples).

HIV-derived peptides were not identified by MS in the urine of subjects with uncontrolled HIV replication, but a clear increase in inflammatory markers and markers unique to HIV-urine were present, potentially offering insight into the pathogenesis and/or monitoring of HIV infection.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

TABLE 1

| | |
|---|---|
| Q8TD57 | 10 20 30 40 50 60 |
| SEQ ID NO: 1 | MGATGRLELT LAAPPHPGPA FQRSKARETQ GEEEGSEMQI AKSDSIHHMS HSQGQPELPP |
| | 70 80 90 100 110 120 |
| | LPASANEEPS GLYQTVMSHS FYPPLMQRTS WTLAAPFKEQ HHHRGPSDSI ANNYSLMAQD |
| | 130 140 150 160 170 180 |
| | LKLKDLLKVY QPATISVPRD RTGQGLPSSG NRSSSEPMRK KTKFSSRNKE DSTRIKLAFK |
| | 190 200 210 220 230 240 |
| | TSIFSPMKKE VKTSLTFPGS RPMSPEQQLD VMLQQEMEME SKEKKPSESD LERYYYYLTN |
| | 250 260 270 280 290 300 |
| | GIRKDMIAPE EGEVMVRISK LISNTLLTSP FLEPLMVVLV QEKENDYYCS LMKSIVDYIL |
| | 310 320 330 340 350 360 |
| | MDPMERKRLF IESIPRLFPQ RVIRAPVPWH SVYRSAKKWN EEHLHTVNPM MLRLKELWFA |
| | 370 380 390 400 410 420 |
| | EFRDLRFVRT AEILAGKLPL QPQEFWDVIQ KHCLEAHQTL LNKWIPTCAQ LFTSRKEHWI |
| | 430 440 450 460 470 480 |
| | HFAPKSNYDS SRNIEEYFAS VASFMSLQLR ELVIKSLEDL VSLFMIHKDG NDFKEPYQEM |
| | 490 500 510 520 530 540 |
| | KFFIPQLIMI KLEVSEPIIV FNPSFDGCWE LIRDSFLEII KNSNGIPKLK YIPLKFSFTA |
| | 550 560 570 580 590 600 |
| | AAADRQCVKA AEPGEPSMHA AATAMAELKG YNLLLGTVNA EEKLVSDFLI QTFKVFQKNQ |
| | 610 620 630 640 650 660 |
| | VGPCKYLNVY KKYVDLLDNT AEQNIAAFLK ENHDIDDFVT KINAIKKRRN EIASMNITVP |
| | 670 680 690 700 710 720 |
| | LAMFCLDATA LNHDLCERAQ NLKDHLIQFQ VDVNRDTNTS ICNQYSHIAD KVSEVPANTK |
| | 730 740 750 760 770 780 |
| | ELVSLIEFLK KSSAVTVFKL RRQLRDASER LEFLMDYADL PYQIEDIFDN SRNLLLHKRD |
| | 790 800 810 820 830 840 |
| | QAEMDLIKRC SEFELRLEGY HRELESFRKR EVMTTEEMKH NVEKLNELSK NLNRAFAEFE |
| | 850 860 870 880 890 900 |
| | LINKEEELLE KEKSTYPLLQ AMLKNKVPYE QLWSTAYEFS IKSEEWMNGP LFLLNAEQIA |
| | 910 920 930 940 950 960 |
| | EEIGNMWRTT YKLIKTLSDV PAPRRLAENV KIKIDKFKQY IPILSISCNP GMKDRHWQQI |
| | 970 980 990 1000 1010 1020 |
| | SEIVGYEIKP TETTCLSNML EFGFGKFVEK LEPIGAAASK EYSLEKNLDR MKLDWVNVTF |
| | 1030 1040 1050 1060 1070 1080 |
| | SFVKYRDTDT NILCAIDDIQ MLLDDHVIKT QTMCGSPFIK PIEAECRKWE EKLIRIQDNL |
| | 1090 1100 1110 1120 1130 1140 |
| | DAWLKCQATW LYLEPIFSSE DIIAQMPEEG RKFGIVDSYW KSLMSQAVKD NRILVAADQP |
| | 1150 1160 1170 1180 1190 1200 |
| | RMAEKLQEAN FLLEDIQKGL NDYLEKKRLF FPRFFFLSND ELLEILSETK DPLRVQPHLK |
| | 1210 1220 1230 1240 1250 1260 |
| | KCFEGIAKLE FTDNLEIVGM ISSEKETVPF IQKIYPANAK GMVEKWLQQV EQMMLASMRE |
| | 1270 1280 1290 1300 1310 1320 |
| | VIGLGIEAYV KVPRNHWVLQ WPGQVVICVS SIFWTQEVSQ ALAENTLLDF LKKSNDQIAQ |
| | 1330 1340 1350 1360 1370 1380 |
| | IVQLVRGKLS SGARLTLGAL TVIDVHARDV VAKLSEDRVS DLNDFQWISQ LRYYWVAKDV |
| | 1390 1400 1410 1420 1430 1440 |
| | QVQIITTEAL YGYEYLGNSP RLVITPLTDR CYRTLMGALK LNLGGAPEGP AGTGKTETTK |
| | 1450 1460 1470 1480 1490 1500 |
| | DLAKALAKQC VVFNCSDGLD YKAMGKFFKG LAQAGAWACF DEFNRIEVEV LSVVAQQILS |
| | 1510 1520 1530 1540 1550 1560 |
| | IQQAIIRKLK TFIFEGTELS LNPTCAVFIT MNPGYAGRAE LPDNLKALFR TVAMMVPDYA |
| | 1570 1580 1590 1600 1610 1620 |
| | LIGEISLYSM GFLDSRSLAQ KIVATYRLCS EQLSSQHHYD YGMRAVKSVL TAAGNLKLKY |
| | 1630 1640 1650 1660 1670 1680 |
| | PEENESVLLL RALLDVNLAK FLAQDVPLFQ GIISDLFPGV VLPKPDYEVF LKVLNDNIKK |
| | 1690 1700 1710 1720 1730 1740 |
| | MKLQPVPWFI GKIIQIYEMM LVRHGYMIVG DPMGGKTSAY KVLAAALGDL HAANQMEEFA |
| | 1750 1760 1770 1780 1790 1800 |
| | VEYKIINPKA ITMGQLYGCF DQVSHEWMDG VLANAFREQA SSLSDDRKWI IFDGPVDAIW |
| | 1810 1820 1830 1840 1850 1860 |
| | IENMNTVLDD NKKLCLMSGE IIQMNSKMSL IFEPADLEQA SPATVSRCGM IYMEPHQLGW |
| | 1870 1880 1890 1900 1910 1920 |
| | KPLKDSYMDT LPSSLTKEHK ELVNDMFMWL VQPCLEFGRL HCKFVVQTSP IHLAFSMMRL |
| | 1930 1940 1950 1960 1970 1980 |
| | YSSLLDEIRA VEEEEMELGE GLSSQQIFLW LQGLFLFSLV WTVAGTINAD SRKKFDVFFR |
| | 1990 2000 2010 2020 2030 2040 |
| | NLIMGMDDNH PRPKSVKLTK NNIFPERGSI YDFYFIKQAS GHWETWTQYI TKEEEKVPAG |
| | 2050 2060 2070 2080 2090 2100 |
| | AKVSELIIPT METARQSFFL KTYLDHEIPM LFVGPTGTGK SAITNNFLLH LPKNTYLPNC |
| | 2110 2120 2130 2140 2150 2160 |
| | INFSARTSAN QTQDIIMSKL DRRRKGLFGP PIGKKAVVFV DDLNMPAKEV YGAQPPIELL |
| | 2170 2180 2190 2200 2210 2220 |
| | RQWIDHGYWF DKKDTTRLDI VDMLLVTAMG PPGGGRNDIT GRFTRHLNII SINAFEDDIL |
| | 2230 2240 2250 2260 2270 2280 |
| | TKIFSSIVDW HFGKGFDVMF LRYGKMLVQA TKTIYRDAVE NFLPTPSKSH YVFNLRDFSR |
| | 2290 2300 2310 2320 2330 2340 |
| | VIQGVLLCPH THLQDVEKCI RLWIHEVYRV FYDRLIDKED RQVFFNMVKE TTSNCFKQTI |
| | 2350 2360 2370 2380 2390 2400 |
| | EKVLIHLSPT GKIVDDNIRS LFFGDYFKPE SDQKIYDEIT DLKQLTVVME HYLEEFNNIS |
| | 2410 2420 2430 2440 2450 2460 |
| | KAPMSLVMFR FAIEHISRIC RVLKQDKGHL LLVGIGGSGR QSAAKLSTFM NAYELYQIEI |
| | 2470 2480 2490 2500 2510 2520 |
| | TKNYAGNDWR EDLKKIILQV GVATKSTVFL FADNQIKDES FVEDINMLLN TGDVPNIFPA |
| | 2530 2540 2550 2560 2570 2580 |
| | DEKADIVEKM QTAARTQGEK VEVTPLSMYN FFIERVINKI SFSLAMSPIG DAFRNRLRMF |
| | 2590 2600 2610 2620 2630 2640 |
| | PSLINCCTID WFQSWPTDAL ELVANKFLED VELDDNIRVE VVSMCKYFQE SVKKLSLDYY |
| | 2650 2660 2670 2680 2690 2700 |
| | NKLRRHNYVT PTSYLELILT FKTLLNSKRQ EVAMMRNRYL TGLQKLDFAA SQVAVMQREL |
| | 2710 2720 2730 2740 2750 2760 |
| | TALQPQLILT SEETAKMMVK IEAETREADG KKLLVQADEK EANVAAAIAQ GIKNECEGDL |
| | 2770 2780 2790 2800 2810 2820 |
| | AEAMPALEAA LAALDTLNPA DISLVKSMQN PPGPVKLVME SICIMKGMKP ERKPDPSGSG |
| | 2830 2840 2850 2860 2870 2880 |
| | KMIEDYWGVS KKILGDLKFL ESLKTYDKDN IPPLTMKRIR ERFINHPEFQ PAVIKNVSSA |
| | 2890 2900 2910 2920 2930 2940 |
| | CEGLCKWVRA MEVYDRVAKV VAPKRERLRE AEGKLAAQMQ KLNQKRAELK LVVDRLQALN |
| | 2950 2960 2970 2980 2990 3000 |
| | DDFEEMNTKK KDLEENIEIC SQKLVRAEKL ISGLGGEKDR WTEAARQLGI RYTNLTGDVL |
| | 3010 3020 3030 3040 3050 3060 |
| | LSSGTVAYLG AFTVDYRVQC QNQWLAECKD KVIPGFSDFS LSHTLGDPIK IRAWQIAGLP |
| | 3070 3080 3090 3100 3110 3120 |
| | VDSFSIDNGI IVSNSRRWAL MIDPHGQANK WIKNMEKANK LAVIKFSDSN YMRMLENALQ |
| | 3130 3140 3150 3160 3170 3180 |
| | LGTPVLIENI GEELDASIEP ILLKATFKQQ GVEYMRLGEN IIEYSRDFKL YITTRLRNPH |
| | 3190 3200 3210 3220 3230 3240 |
| | YLPEVAVKVC LLNFMITPLG LQDQLLGIVA AKEKPELEEK KNQLIVESAK NKKHLKEIED |
| | 3250 3260 3270 3280 3290 3300 |
| | KILEVLSMSK GNILEDETAI KVLSSSKVLS EEISEKQKVA SMTETQIDET RMGYKPVAVH |
| | 3310 3320 3330 3340 3350 3360 |
| | SATIFFCISD LANIEPMYQY SLTWFINLYM HSLTHSTKSE ELNLRIKYII DHFTLSIYNN |
| | 3370 3380 3390 3400 3410 3420 |
| | VCRSLFEKDK LLFSLLLTIG IMKQKKEITE EVWYFLLTGG IALDNPYPNP APQWLSEKAW |
| | 3430 3440 3450 3460 3470 3480 |
| | AEIVRASALP KLHGLMEHLE QNLGEWKLIY DSAWPHEEQL PGSWKFSQGL EKMVILRCLR |
| | 3490 3500 3510 3520 3530 3540 |
| | PDKMVPAVRE FIAEHMGKLY IEAPTFDLQG SYNDSSCCAP LIFVLSPSAD PMAGLLKFAD |
| | 3550 3560 3570 3580 3590 3600 |
| | DLGMGGTRTQ TISLGQGQGP IAAKMINNAI KDGTWVVLQN CHLAASWMPT LEKICEEVIV |
| | 3610 3620 3630 3640 3650 3660 |
| | PESTNARFRL WLTSYPSEKF PVSILQNGIK MTNEPPKGLR ANLLRSYLND PISDPVFFQS |
| | 3670 3680 3690 3700 3710 3720 |
| | CAKAVMWQKM LFGLCFFHAV VQERRNFGPL GWNIPYEFNE SDLRISMWQI QMFLNDYKEV |
| | 3730 3740 3750 3760 3770 3780 |
| | PFDALTYLTG ECNYGGRVTD DKDRRLLLSL LSMFYCKEIE EDYYSLAPGD TYYIPPHGSY |
| | 3790 3800 3810 3820 3830 3840 |
| | QSYIDYLRNL PITAHPEVFG LHENADITKD NQETNQLFEG VLLTLPRQSG GSGKSPQEVV |
| | 3850 3860 3870 3880 3890 3900 |
| | EELAQDILSK LPRDFDLEEV MKLYPVVYEE SMNTVLRQEL IRFNRLTKVV RRSLINLGRA |
| | 3910 3920 3930 3940 3950 3960 |
| | IKGQVLMSSE LEEVFNSMLV GKVPAMWAAK SYPSLKPLGG YVADLLARLT FFQEWIDKGP |
| | 3970 3980 3990 4000 4010 4020 |
| | PVVFWISGFY FTQSFLTGVS QNYARKYTIP IDHIGFEFEV TPQETVMENN PEDGAYIKGL |
| | 4030 4040 4050 4060 4070 4080 |
| | FLEGARWDRK TMQIGESLPK ILYDPLPIIW LKPGESAMFL HQDIYVCPVY KTSARRGTLS |
| | 4090 4100 4110 |
| | TTGHSTNYVL SIELPTDMPQ KHWINRGVAS LCQLDN |
| Q18PE1 | 10 20 30 40 50 60 |
| SEQ ID NO: 2 | MTEAALVEGQ VKLRDGKKWK SRWLVLRKPS PVADCLLMLV YKDKSERIKG LRERSSLTLE |
| | 70 80 90 100 110 120 |
| | DICGLEPGLP YEGLVHTLAI VCLSQAIMLG FDSHEAMCAW DARIRYALGE VHRFHVTVAP |
| | 130 140 150 160 170 180 |
| | GTKLESGPAT LHLCNDVLVL ARDIPPAVTG QWKLSDLRRY GAVPSGFIFE GGTRCGYWAG |
| | 190 200 210 220 230 240 |
| | VFFLSSAEGE QISFLFDCIV RGISPTKGPF GLRPVLPDPS PPGPSTVEER VAQEALETLQ |
| | 250 260 270 280 290 300 |
| | LEKRLSLLSH AGRPGSGGDD RSLSSSSSEA SHLDVSASSR LTAWPEQSSS SASTSQEGPR |
| | 310 320 330 340 350 360 |
| | PAAAQAAGEA MVGASRPPPK PLRPRQLQEV GRQSSSDSGI ATGSHSSYSS SLSSYAGSSL |
| | 370 380 390 400 410 420 |
| | DVWRATDELG SLLSLPAAGA PEPSLCTCLP GTVEYQVPTS LRAHYDTPRS LCLAPRDHSP |
| | 430 440 450 460 470 480 |
| | PSQGSPGNSA ARDSGGQTSA GCPSGWLGTR RRGLVMEAPQ GSEATLPGPA PGEPWEAGGP |
| | 490 500 |
| | HAGPPPAFFS ACPVCGGLKV NPPP |
| Q8NFH5 | 10 20 30 40 50 60 |
| SEQ ID NO: 3 | MAAFAVEPQG PALGSEPMML GSPTSPKPGV NAQFLPGFLM GDLPAPVTPQ PRSISGPSVG |
| | 70 80 90 100 110 120 |
| | VMEMRSPLLA GGSPPQPVVP AHKDKSGAPP VRSIYDDISS PGLGSTPLTS RRQPNISVMQ |
| | 130 140 150 160 170 180 |
| | SPLVGVTSTP GTGQSMFSPA SIGQPRKTTL SPAQLDPFYT QGDSLTSEDH LDDSWVTVFG |
| | 190 200 210 220 230 240 |
| | FPQASASYIL LQFAQYGNIL KHVMSNTGNW MHIRYQSKLQ ARKALSKDGR IFGESIMIGV |
| | 250 260 270 280 290 300 |
| | KPCIDKSVME SSDRCALSSP SLAFTPPIKT LGTPTQPGST PRISTMRPLA TAYKASTSDY |
| | 310 320 |
| | QVISDRQTPK KDESLVSKAM EYMFGW |
| Q8WYL5 | 10 20 30 40 50 60 |
| SEQ ID NO: 4 | MALVTLQRSP TPSAASSSAS NSELEAGSEE DRKLNLSLSE SFFMVKGAAL FLQQGSSPQG |
| | 70 80 90 100 110 120 |
| | QRSLQHPHKH AGDLPQHLQV MINLLRCEDR IKLAVRLESA WADRVRYMVV VYSSGRQDTE |
| | 130 140 150 160 170 180 |
| | ENILLGVDFS SKESKSCTIG MVLRLWSDTK IHLDGDGGFS VSTAGRMHIF KPVSVQAMWS |
| | 190 200 210 220 230 240 |
| | ALQVLHKACE VARRHNYFPG GVALIWATYY ESCISSEQSC INEWNAMQDL ESTRPDSPAL |
| | 250 260 270 280 290 300 |
| | FVDKPTEGER TERLIKAKLR SIMMSQDLEN VTSKEIRNEL EKQMNCNLKE LKEFIDNEML |
| | 310 320 330 340 350 360 |
| | LILGQMDKPS LIFDHLYLGS EWNASNLEEL QGSGVDYILN VTREIDNFFP GLFAYHNIRV |
| | 370 380 390 400 410 420 |
| | YDEETTDLLA HWNEAYHFIN KAKRNHSKCL VHCKMGVSRS ASTVIAYAMK EFGWPLEKAY |
| | 430 440 450 460 470 480 |
| | NYVKQKRSIT RPNAGFMRQL SEYEGILDAS KQRHNKLWRQ QTDSSLQQPV DDPAGPGDFL |
| | 490 500 510 520 530 540 |
| | PETPDGTPES QLPFLDDAAQ PGLGPPLPCC FRRLSDPLLP SPEDETGSLV HLEDPEREAL |
| | 550 560 570 580 590 600 |
| | LEEAAPPAEV HRPARQPQQG SGLCEKDVKK KLEFGSPKGR SGSLLQVEET EREEGLGAGR |
| | 610 620 630 640 650 660 |
| | WGQLPTQLDQ NLLNSENLNN NSKRSCPNGM EDDAIFGILN KVKPSYKSCA DCMYPTASGA |
| | 670 680 690 700 710 720 |
| | PEASRERCED PNAPAICTQP AFLPHITSSP VAHLASRSRV PEKPASGPTE PPPFLPPAGS |
| | 730 740 750 760 770 780 |
| | RRADTSGPGA GAALEPPASL LEPSRETPKV LPKSLLLKNS HCDKNPPSTE VVIKEESSPK |
| | 790 800 810 820 830 840 |
| | KDMKPAKDLR LLFSNESEKP TTNSYLMQHQ ESIIQLQKAG LVRKHTKELE RLKSVPADPA |
| | 850 860 870 880 890 900 |
| | PPSRDGPASR LEASIPEESQ DPAALHELGP LVMPSQAGSD EKSEAAPASL EGGSLKSPPP |
| | 910 920 930 940 950 960 |
| | FFYRLDHTSS FSKDFLKTIC YTPTSSSMSS NLTRSSSSDS IHSVRGKPGL VKQRTQEIET |
| | 970 980 990 1000 1010 1020 |
| | RLRLAGLTVS SPLKRSHSLA KLGSLTFSTE DLSSEADPST VADSQDTTLS ESSFLHEPQG |
| | 1030 1040 |
| | TPRDPAATSK PSGKPAPENL KSPSWMSKS |
| Q8IYD8 | 10 20 30 40 50 60 |
| SEQ ID NO: 5 | MSGRQRTLFQ TWGSSISRSS GTPGCSSGTE RPQSPGSSKA PLPAAAEAQL ESDDDVLLVA |
| | 70 80 90 100 110 120 |
| | AYEAERQLCL ENGGFCTSAG ALWIYPTNCP VRDYQLHISR AALFCNTLVC LPTGLGKTFI |
| | 130 140 150 160 170 180 |
| | AAVVMYNFYR WFPSGKVVFM APTKPLVTQQ IEACYQVMGI PQSHMAEMTG STQASTRKEI |
| | 190 200 210 220 230 240 |
| | WCSKRVLFLT PQVMVNDLSR GACPAAEIKC LVIDEAHKAL GNYAYCQVVR ELVKYTNHFR |
| | 250 260 270 280 290 300 |
| | ILALSATPGS DIKAVQQVIT NLLIGQIELR SEDSPDILTY SHERKVEKLI VPLGEELAAI |
| | 310 320 330 340 350 360 |
| | QKTYIQILES FARSLIQRNV LMRRDIPNLT KYQIILARDQ FRKNPSPNIV GIQQGIIEGE |
| | 370 380 390 400 410 420 |
| | FAICISLYHG YELLQQMGMR SLYFFLCGIM DGTKGMTRSK NELGRNEDFM KLYNHLECMF |
| | 430 440 450 460 470 480 |
| | ARTRSTSANG ISAIQQGDKN KKFVYSHPKL KKLEEVVIEH FKSWNAENTT EKKRDETRVM |
| | 490 500 510 520 530 540 |
| | IFSSFRDSVQ EIAEMLSQHQ PIIRVMTFVG HASGKSTKGF TQKEQLEVVK QFRDGGYNTL |
| | 550 560 570 580 590 600 |
| | VSTCVGEEGL DIGEVDLIIC FDSQKSPIRL VQRMGRTGRK RQGRIVIILS EGREERIYNQ |
| | 610 620 630 640 650 660 |
| | SQSNKRSIYK AISSNRQVLH FYQRSPRMVP DGINPKLHKM FITHGVYEPE KPSRNLQRKS |
| | 670 680 690 700 710 720 |
| | SIFSYRDGMR QSSLKKDWFL SEEEFKLWNR LYRLRDSDEI KEITLPQVQF SSLQNEENKP |
| | 730 740 750 760 770 780 |
| | AQESTTGIHQ LSLSEWRLWQ DHPLPTHQVD HSDRCRHFIG LMQMIEGMRH EEGECSYELE |
| | 790 800 810 820 830 840 |
| | VESYLQMEDV TSTFIAPRNE SNNLASDTFI THKKSSFIKN INQGSSSSVI ESDEECAEIV |
| | 850 860 870 880 890 900 |
| | KQTHIKPTKI VSLKKKVSKE IKKDQLKKEN NHGIIDSVDN DRNSTVENIF QEDLPNDKRT |
| | 910 920 930 940 950 960 |
| | SDTDEIAATC TINENVIKEP CVLLTECQFT NKSTSSLAGN VLDSGYNSFN DEKSVSSNLF |
| | 970 980 990 1000 1010 1020 |
| | LPFEEELYIV RTDDQFYNCH SLTKEVLANV ERFLSYSPPP LSGLSDLEYE IAKGTALENL |
| | 1030 1040 1050 1060 1070 1080 |
| | LFLPCAEHLR SDKCTCLLSH SAVNSQQNLE LNSLKCINYP SEKSCLYDIP NDNISDEPSL |
| | 1090 1100 1110 1120 1130 1140 |
| | CDCDVHKHNQ NENLVPNNRV QIHRSPAQNL VGENNHDVDN SDLPVLSTDQ DESLLLFEDV |
| | 1150 1160 1170 1180 1190 1200 |
| | NTEFDDVSLS PLNSKSESLP VSDKTAISET PLVSQFLISD ELLLDNNSEL QDQITRDANS |
| | 1210 1220 1230 1240 1250 1260 |
| | FKSRDQRGVQ EEKVKNHEDI FDCSRDLFSV TFDLGFCSPD SDDEILEHTS DSNRPLDDLY |
| | 1270 1280 1290 1300 1310 1320 |
| | GRYLEIKEIS DANYVSNQAL IPRDHSKNFT SGTVIIPSNE DMQNPNYVHL PLSAAKNEEL |
| | 1330 1340 1350 1360 1370 1380 |
| | LSPGYSQFSL PVQKKVMSTP LSKSNTLNSF SKIRKEILKT PDSSKEKVNL QRFKEALNST |
| | 1390 1400 1410 1420 1430 1440 |
| | FDYSEFSLEK SKSSGPMYLH KSCHSVEDGQ LLTSNESEDD EIFRRKVKRA KGNVLNSPED |
| | 1450 1460 1470 1480 1490 1500 |
| | QKNSEVDSPL HAVKKRRFPI NRSELSSSDE SENFPKPCSQ LEDFKVCNGN ARRGIKVPKR |
| | 1510 1520 1530 1540 1550 1560 |
| | QSHLKHVARK FLDDEAELSE EDAEYVSSDE NDESENEQDS SLLDFLNDET QLSQAINDSE |
| | 1570 1580 1590 1600 1610 1620 |
| | MRAIYMKSLR SPMMNNKYKM IHKTHKNINI FSQIPEQDET YLEDSFCVDE EESCKGQSSE |
| | 1630 1640 1650 1660 1670 1680 |
| | EEVCVDFNLI TDDCFANSKK YKTRRAVMLK EMMEQNCAHS KKKLSRIILP DDSSEEENNV |
| | 1690 1700 1710 1720 1730 1740 |
| | NDKRESNIAV NPSTVKKNKQ QDHCLNSVPS GSSAQSKVRS TPRVNPLAKQ SKQTSLNLKD |
| | 1750 1760 1770 1780 1790 1800 |
| | TISEVSDFKP QNHNEVQSTT PPFTTVDSQK DCRKFPVPQK DGSALEDSST SGASCSKSRP |
| | 1810 1820 1830 1840 1850 1860 |
| | HLAGTHTSLR LPQEGKGTCI LVGGHEITSG LEVISSLRAI HGLQVEVCPL NGCDYIVSNR |
| | 1870 1880 1890 1900 1910 1920 |
| | MVVERRSQSE MLNSVNKNKF IEQIQHLQSM FERICVIVEK DREKTGDTSR MFRRTKSYDS |
| | 1930 1940 1950 1960 1970 1980 |
| | LLTTLIGAGI RILFSSCQEE TADLLKELSL VEQRKNVGIH VPTVVNSNKS EALQFYLSIP |
| | 1990 2000 2010 2020 2030 2040 |
| | NISYITALNM CHQFSSVKRM ANSSLQEISM YAQVTHQKAE EIYRYIHYVF DIQMLPNDLN |
| | QDRLKSDI |
| O14654 | 10 20 30 40 50 60 |
| SEQ ID NO: 6 | MASCSFTRDQ ATRRLRGAAA AAAAALAAVV TTPLLSSGTP TALIGTGSSC PGAMWLSTAT |
| | 70 80 90 100 110 120 |
| | GSRSDSESEE EDLPVGEEVC KRGYLRKQKH GHRRYFVLKL ETADAPARLE YYENARKFRH |
| | 130 140 150 160 170 180 |
| | SVRAAAAAAA AAASGAAIPP LIPPRRVITL YQCFSVSQRA DARYRHLIAL FTQDEYFAMV |
| | 190 200 210 220 230 240 |
| | AENESEQESW YLLLSRLILE SKRRRCGTLG AQPDGEPAAL AAAAAAEPPF YKDVWQVIVK |
| | 250 260 270 280 290 300 |
| | PRGLGHRKEL SGVFRLCLTD EEVVFVRLNT EVASVVVQLL SIRRCGHSEQ YFFLEVGRST |
| | 310 320 330 340 350 360 |
| | VIGPGELWMQ VDDCVVAQNM HELFLEKMRA LCADEYRARC RSYSISIGAH LLTLLSARRH |
| | 370 380 390 400 410 420 |
| | LGLVPLEPGG WLRRSRFEQF CHLRAIGDGE DEMLFTRRFV TPSEPVAHSR RGRLHLPRGR |
| | 430 440 450 460 470 480 |
| | RSRRAVSVPA SFFRRLAPSP ARPRHPAEAP NNGARLSSEV SGSGSGNFGE EGNPQGKEDQ |
| | 490 500 510 520 530 540 |
| | EGSGGDYMPM NNWGSGNGRG SGGGQGSNGQ GSSSHSSGGN QCSGEGQGSR GGQGSNGQGS |
| | 550 560 570 580 590 600 |
| | GGNQCSRDGQ GTAGGHGSGG GQRPGGGHGS GGGQGPGDGH GSGGGKNSGG GKGSGSGKGS |
| | 610 620 630 640 650 660 |
| | DGDGERGKSL KKRSYFGKLT QSKQQQMPPP PPPPPPPPPA GGTGGKGKSG GRFRLYFCVD |
| | 670 680 690 700 710 720 |
| | RGATKECKEA KEVKDAEIPE GAARGPHRAR AFDEDEDDPY VPMRPGVATP LVSSSDYMPM |
| | 730 740 750 760 770 780 |
| | APQNVSASKK RHSRSPFEDS RGYMMMFPRV SPPPAPSPPK APDTNKEDDS KDNDSESDYM |
| | 790 800 810 820 830 840 |
| | FMAPGAGAIP KNPRNPQGGS SSKSWSSYFS LPNPFRSSPL GQNDNSEYVP MLPGKFLGRG |
| | 850 860 870 880 890 900 |
| | LDKEVSYNWD PKDAASKPSG EGSFSKPGDG GSPSKPSDHE PPKNKAKRPN RLSFITKGYK |
| | 910 920 930 940 950 960 |
| | IKPKPQKPTH EQREADSSSD YVNMDFTKRE SNTPAPSTQG LPDSWGIIAE PRQSAFSNYV |
| | 970 980 990 1000 1010 1020 |
| | NVEFGVPFPN PANDLSDLLR AIPRANPLSL DSARWPLPPL PLSATGSNAI EEEGDYIEVI |
| | 1030 1040 1050 1060 1070 1080 |
| | FNSAMTPAMA LADSAIRYDA ETGRIYVVDP FSECCMDISL SPSRCSEPPP VARLLQEEEQ |
| | 1090 1100 1110 1120 1130 1140 |
| | ERRRPQSRSQ SFFAAARAAV SAFPTDSLER DLSPSSAPAV ASAAEPTLAL SQVVAAASAL |
| | 1150 1160 1170 1180 1190 1200 |
| | AAAPGIGAAA AAAGFDSASA RWFQPVANAA DAEAVRGAQD VAGGSNPGAH NPSANLARGD |
| | 1210 1220 1230 1240 1250 |
| | NQAGGAAAAA AAPEPPPRSR RVPRPPERED SDNDDDTHVR MDFARRDNQF DSPKRGR |
| Q96AP4 | 10 20 30 40 50 60 |
| SEQ ID NO: 7 | MLSCNICGET VTSEPDMKAH LIVHMESEII CPFCKLSGVN YDEMCFHIET AHFEQNTLER |
| | 70 80 90 100 110 120 |
| | NFERINTVQY GTSDNKKDNT LQCGMEVNSS ILSGCASNHP KNSAQNLTKD STLKHEGFYS |
| | 130 140 150 160 170 180 |
| | ENLTESRKFL KSREKQSSLT EIKGSVYETT YSPPECPFCG KIEEHSEDME THVKTKHANL |
| | 190 200 210 220 230 240 |
| | LDIPLEDCDQ PLYDCPMCGL ICTNYHILQE HVDLHLEENS FQQGMDRVQC SGDLQLAHQL |
| | 250 260 270 280 290 300 |
| | QQEEDRKRRS EESRQEIEEF QKLQRQYGLD NSGGYKQQQL RNMEIEVNRG RMPPSEFHRR |
| | 310 320 330 340 350 360 |
| | KADMMESLAL GFDDGKTKTS GIIEALHRYY QNAATDVRRV WLSSVVDHFH SSLGDKGWGC |
| | 370 380 390 400 410 420 |
| | GYRNFQMLLS SLLQNDAYND CLKGMLIPCI PKIQSMIEDA WKEGFDPQGA SQLNNRLQGT |
| | 430 440 450 460 470 480 |
| | KAWIGACEVY ILLTSLRVKC HIVDFHKSTG PLGTHPRLFE WILNYYSSEG EGSPKVVCTS |
| | 490 500 510 520 530 540 |
| | KPPIYLQHQG HSRTVIGIEE KKNRTLCLLI LDPGCPSREM QKLLKQDIEA SSLKQLRKSM |
| | 550 560 570 |
| | GNLKHKQYQI LAVEGALSLE EKLARRQASQ VFTAEKIP |
| Q9UQ35 | 10 20 30 40 50 60 |
| SEQ ID NO: 8 | MYNGIGLPTP RGSGTNGYVQ RNLSLVRGRR GERPDYKGEE ELRRLEAALV KRPNPDILDH |
| | 70 80 90 100 110 120 |
| | ERKRRVELRC LELEEMMEEQ GYEEQQIQEK VATFRLMLLE KDVNPGGKEE TPGQRPAVTE |
| | 130 140 150 160 170 180 |
| | THQLAELNEK KNERLRAAFG ISDSYVDGSS FDPQRRAREA KQPAPEPPKP YSLVRESSSS |
| | 190 200 210 220 230 240 |
| | RSPTPKQKKK KKKKDRGRRS ESSSPRRERK KSSKKKKHRS ESESKKRKHR SPTPKSKRKS |
| | 250 260 270 280 290 300 |
| | KDKKRKRSRS TTPAPKSRRA HRSTSADSAS SSDTSRSRSR SAAAKTHTTA LAGRSPSPAS |
| | 310 320 330 340 350 360 |
| | GRRGEGDAPF SEPGTTSTQR PSSPETATKQ PSSPYEDKDK DKKEKSATRP SPSPERSSTG |
| | 370 380 390 400 410 420 |
| | PEPPAPTPLL AERHGGSPQP LATTPLSQEP VNPPSEASPT RDRSPPKSPE KLPQSSSSES |
| | 430 440 450 460 470 480 |
| | SPPSPQPTKV SRHASSSPES PKPAPAPGSH REISSSPTSK NRSHGRAKRD KSHSHTPSRR |
| | 490 500 510 520 530 540 |
| | MGRSRSPATA KRGRSRSRTP TKRGHSRSRS PQWRRSRSAQ RWGRSRSPQR RGRSRSPQRP |
| | 550 560 570 580 590 600 |
| | GWSRSRNTQR RGRSRSARRG RSHSRSPATR GRSRSRTPAR RGRSRSRTPA RRRSRSRTPT |
| | 610 620 630 640 650 660 |
| | RRRSRSRTPA RRGRSRSRTP ARRRSRTRSP VRRRSRSRSP ARRSGRSRSR TPARRGRSRS |
| | 670 680 690 700 710 720 |
| | RTPARRGRSR SRTPARRSGR SRSRTPARRG RSRSRTPRRG RSRSRSLVRR GRSHSRTPQR |
| | 730 740 750 760 770 780 |
| | RGRSGSSSER KNKSRTSQRR SRSNSSPEMK KSRISSRRSR SLSSPRSKAK SRLSLRRSLS |
| | 790 800 810 820 830 840 |
| | GSSPCPKQKS QTPPRRSRSG SSQPKAKSRT PPRRSRSSSS PPPKQKSKTP SRQSHSSSSP |
| | 850 860 870 880 890 900 |
| | HPKVKSGTPP RQGSITSPQA NEQSVTPQRR SCFESSPDPE LKSRTPSRHS CSGSSPPRVK |
| | 910 920 930 940 950 960 |
| | SSTPPRQSPS RSSSPQPKVK AIISPRQRSH SGSSSPSPSR VTSRTTPRRS RSVSPCSNVE |
| | 970 980 990 1000 1010 1020 |
| | SRLLPRYSHS GSSSPDTKVK PETPPRQSHS GSISPYPKVK AQTPPGPSLS GSKSPCPQEK |
| | 1030 1040 1050 1060 1070 1080 |
| | SKDSLVQSCP GSLSLCAGVK SSTPPGESYF GVSSLQLKGQ SQTSPDHRSD TSSPEVRQSH |
| | 1090 1100 1110 1120 1130 1140 |
| | SESPSLQSKS QTSPKGGRSR SSSPVTELAS RSPIRQDRGE FSASPMLKSG MSPEQSRFQS |
| | 1150 1160 1170 1180 1190 1200 |
| | DSSSYPTVDS NSLLGQSRLE TAESKEKMAL PPQEDATASP PRQKDKFSPF PVQDRPESSL |
| | 1210 1220 1230 1240 1250 1260 |
| | VFKDTLRTPP RERSGAGSSP ETKEQNSALP TSSQDEELME VVEKSEEPAG QILSHLSSEL |
| | 1270 1280 1290 1300 1310 1320 |
| | KEMSTSNFES SPEVEERPAV SLTLDQSQSQ ASLEAVEVPS MASSWGGPHF SPEHKELSNS |
| | 1330 1340 1350 1360 1370 1380 |
| | PLRENSFGSP LEFRNSGPLG TEMNTGFSSE VKEDLNGPFL NQLETDPSLD MKEQSTRSSG |
| | 1390 1400 1410 1420 1430 1440 |
| | HSSSELSPDA VEKAGMSSNQ SISSPVLDAV PRTPSRERSS SASSPEMKDG LPRTPSRRSR |
| | 1450 1460 1470 1480 1490 1500 |
| | SGSSPGLRDG SGTPSRHSLS GSSPGMKDIP RTPSRGRSEC DSSPEPKALP QTPRPRSRSP |
| | 1510 1520 1530 1540 1550 1560 |
| | SSPELNNKCL TPQRERSGSE SSVDQKTVAR TPLGQRSRSG SSQELDVKPS ASPQERSESD |
| | 1570 1580 1590 1600 1610 1620 |
| | SSPDSKAKTR TPLRQRSRSG SSPEVDSKSR LSPRRSRSGS SPEVKDKPRA APRAQSGSDS |
| | 1630 1640 1650 1660 1670 1680 |
| | SPEPKAPAPR ALPRRSRSGS SSKGRGPSPE GSSSTESSPE HPPKSRTARR GSRSSPEPKT |
| | 1690 1700 1710 1720 1730 1740 |
| | KSRTPPRRRS SRSSPELTRK ARLSRRSRSA SSSPETRSRT PPRHRRSPSV SSPEPAEKSR |
| | 1750 1760 1770 1780 1790 1800 |
| | SSRRRRSASS PRTKTTSRRG RSPSPKPRGL QRSRSRSRRE KTRTTRRRDR SGSSQSTSRR |
| | 1810 1820 1830 1840 1850 1860 |
| | RQRSRSRSRV TRRRRGGSGY HSRSPARQES SRTSSRRRRG RSRTPPTSRK RSRSRTSPAP |
| | 1870 1880 1890 1900 1910 1920 |
| | WKRSRSRASP ATHRRSRSRT PLISRRRSRS RTSPVSRRRS RSRTSVTRRR SRSRASPVSR |
| | 1930 1940 1950 1960 1970 1980 |
| | RRSRSRTPPV TRRRSRSRTP TTRRRSRSRT PPVTRRRSRS RTPPVTRRRS RSRTSPITRR |
| | 1990 2000 2010 2020 2030 2040 |
| | RSRSRTSPVT RRRSRSRTSP VTRRRSRSRT SPVTRRRSRS RTPPAIRRRS RSRTPLLPRK |
| | 2050 2060 2070 2080 2090 2100 |
| | RSRSRSPLAI RRRSRSRTPR TARGKRSLTR SPPAIRRRSA SGSSSDRSRS ATPPATRNHS |
| | 2110 2120 2130 2140 2150 2160 |
| | GSRTPPVALN SSRMSCFSRP SMSPTPLDRC RSPGMLEPLG SSRTPMSVLQ QAGGSMMDGP |
| | 2170 2180 2190 2200 2210 2220 |
| | GPRIPDHQRT SVPENHAQSR IALALTAISL GTARPPPSMS AAGLAARMSQ VPAPVPLMSL |
| | 2230 2240 2250 2260 2270 2280 |
| | RTAPAANLAS RIPAASAAAM NLASARTPAI PTAVNLADSR TPAAAAAMNL ASPRTAVAPS |
| | 2290 2300 2310 2320 2330 2340 |
| | AVNLADPRTP TAPAVNLAGA RTPAALAALS LTGSGTPPTA ANYPSSSRTP QAPASANLVG |
| | 2350 2360 2370 2380 2390 2400 |
| | PRSAHATAPV NIAGSRTAAA LAPASLTSAR MAPALSGANL TSPRVPLSAY ERVSGRTSPP |
| | 2410 2420 2430 2440 2450 2460 |
| | LLDRARSRTP PSAPSQSRMT SERAPSPSSR MGQAPSQSLL PPAQDQPRSP VPSAFSDQSR |
| | 2470 2480 2490 2500 2510 2520 |
| | CLIAQTTPVA GSQSLSSGAV ATTTSSAGDH NGMLSVPAPG VPHSDVGEPP ASTGAQQPSA |
| | 2530 2540 2550 2560 2570 2580 |
| | LAALQPAKER RSSSSSSSSS SSSSSSSSSS SSSSSSGSSS SDSEGSSLPV QPEVALKRVP |
| | 2590 2600 2610 2620 2630 2640 |
| | SPTPAPKEAV REGRPPEPTP AKRKRRSSSS SSSSSSSSSS SSSSSSSSSS SSSSSSSSSS |
| | 2650 2660 2670 2680 2690 2700 |
| | SSSSSSSSPS PAKPGPQALP KPASPKKPPP GERRSRSPRK PIDSLRDSRS LSYSPVERRR |
| | 2710 2720 2730 2740 2750 |
| | PSPQPSPRDQ QSSSSERGSR RGQRGDSRSP SHKRRRETPS PRPMRHRSSR SP |
| Q8N6W0 | 10 20 30 40 50 60 |
| SEQ ID NO: 9 | MARLTESEAR RQQQQLLQPR PSPVGSSGPE PPGGQPDGMK DLDAIKLFVG QIPRHLDEKD |
| | 70 80 90 100 110 120 |
| | LKPLFEQFGR IYELTVLKDP YTGMHKGCAF LTYCARDSAI KAQTALHEQK TLPGMARPIQ |
| | 130 140 150 160 170 180 |
| | VKPADSESRG GRDRKLFVGM LNKQQSEEDV LRLFQPFGVI DECTVLRGPD GSSKGCAFVK |
| | 190 200 210 220 230 240 |
| | FSSHTEAQAA IHALHGSQTM PGASSSLVVK FADTDKERTL RRMQQMVGQL GILTPSLTLP |
| | 250 260 270 280 290 300 |
| | FSPYSAYAQA LMQQQTTVLS TSGSYLSPGV AFSPCHIQQI GAVSLNGLPA TPIAPASGLH |
| | 310 320 330 340 350 360 |
| | SPPLLGTTAV PGLVAPITNG FAGVVPFPGG HPALETVYAN GLVPYPAQSP TVAETLHPAF |
| | 370 380 390 400 410 420 |
| | SGVQQYTAMY PTAAITPIAH SVPQPPPLLQ QQQREGPEGC NLFIYHLPQE FGDTELTQMF |
| | 430 440 450 460 470 480 |
| | LPFGNIISSK VFMDRATNQS KCFGFVSFDN PASAQAAIQA MNGFQIGMKR LKVQLKRPKD |
| | PGHPY |
| Q911792 | 10 20 30 40 50 60 |
| SEQ ID NO: 10 | MSACNTFTEH VWKPGECKNC FKPKSLHQLP PDPEKAPITH GNVKTNANHS NNHRIRNTGN |
| | 70 80 90 100 110 120 |
| | FRPPVAKKPT IAVKPTMIVA DGQSICGELS IQEHCENKPV IIGWNRNRAA LSQKPLNNNN |
| | 130 140 150 160 170 180 |
| | EDDEGISHVP KPYGNNDSAK KMSDNNNGLT EVLKEIAGLD TAPQIRGNET NSRETFLGRI |
| | 190 200 210 220 230 240 |
| | NDCYKRSLER KLPPSCMIGG IKETQGKHVI LSGSTEVISN EGGRFCYPEF SSGEESEEDV |
| | 250 260 270 280 290 300 |
| | LFSNMEEEHE SWDESDEELL AMEIRMRGQP RFANFRANTL SPVRFFVDKK WNTIPLRNKS |
| | 310 320 330 340 350 360 |
| | LQRICAVDYD DSYDEILNGY EENSVVSYGQ GSIQSMVSSD STSPDSSLTE ESRSETASSL |
| | 370 380 390 400 410 420 |
| | SQKICNGGLS PGNPGDSKDM KEIEPNYESP SSNNQDKDSS QASKSSIKVP ETHKAVLALR |
| | 430 440 450 460 470 480 |
| | LEEKDGKIAV QTEKEESKAS TDVAGQAVTI NLVPTEEQAK PYRVVNLEQP LCKPYTVVDV |
| | 490 500 510 520 530 540 |
| | SAAMASEHLE GPVNSPKTKS SSSTPNSPVT SSSLTPGQIS AHFQKSSAIR YQEVWTSSTS |
| | 550 560 570 580 590 600 |
| | PRQKIPKVEL ITSGTGPNVP PRKNCHKSAP TSPTATNISS KTIPVKSPNL SEIKFNSYNN |
| | 610 620 630 640 650 660 |
| | AGMPPFPIII HDEPTYARSS KNAIKVPIVI NPNAYDNLAI YKSFLGTSGE LSVKEKTTSV |
| | 670 680 690 700 710 720 |
| | ISHTYEEIET ESKVPDNTTS KTTDCLQTKG FSNSTEHKRG SVAQKVQEFN NCLNRGQSSP |
| | 730 740 750 760 770 780 |
| | QRSYSSSHSS PAKIQRATQE PVAKIEGTQE SQMVGSSSTR EKASTVLSQI VASIQPPQSP |
| | 790 800 810 820 830 840 |
| | PETPQSGPKA CSVEELYAIP PDADVAKSTP KSTPVRPKSL FTSQPSGEAE APQTTDSPTT |
| | 850 860 870 880 890 900 |
| | KVQKDPSIKP VTPSPSKLVT SPQSEPPAPF PPPRSTSSPY HAGNLLQRHF TNWTKPTSPT |
| | 910 920 930 940 950 960 |
| | RSTEAESVLH SEGSRRAADA KPKRWISFKS FFRRRKTDEE DDKEKEREKG KLVGLDGTVI |
| | 970 980 990 1000 1010 1020 |
| | HMLPPPPVQR HHWFTEAKGE SSEKPAIVFM YRCDPAQGQL SVDQSKARTD QAAVMEKGRA |
| | 1030 1040 1050 1060 1070 1080 |
| | ENALLQDSEK KRSHSSPSQI PKKILSHMTH EVTEDFSPRD PRTVVGKQDG RGCTSVTTAL |
| | 1090 1100 1110 1120 1130 1140 |
| | SLPELEREDG KEDISDPMDP NPCSATYSNL GQSRAAMIPP KQPRQPKGAV DDAIAFGGKT |
| | 1150 1160 1170 1180 1190 1200 |
| | DQEAPNASQP TPPPLPKKMI IRANTEPISK DLQKSMESSL CVMANPTYDI DPNWDASSAG |
| | 1210 1220 1230 1240 1250 1260 |
| | SSISYELKGL DIESYDSLER PLRKERPVPS AANSISSLTT LSIKDRFSNS MESLSSRRGP |
| | 1270 1280 1290 1300 1310 1320 |
| | SCRQGRGIQK PQRQALYRGL ENREEVVGKI RSLHTDALKK LAVKCEDLFM AGQKDQLRFG |
| | 1330 1340 1350 1360 1370 1380 |
| | VDSWSDFRLT SDKPCCEAGD AVYYTASYAK DPLNNYAVKI CKSKAKESQQ YYHSLAVRQS |
| | 1390 1400 1410 1420 1430 1440 |
| | LAVHFNIQQD CGHFLAEVPN RLLPWEDPDD PEKDEDDMEE TEEDAKGETD GKNPKPCSEA |
| | 1450 1460 1470 1480 1490 1500 |
| | ASSQKENQGV MSKKQRSHVV VITREVPCLT VADFVRDSLA QHGKSPDLYE RQVCLLLLQL |
| | 1510 1520 1530 1540 1550 1560 |
| | CSGLEHLKPY HVTHCDLRLE NLLLVHYQPG GTAQGFGPAE PSPTSSYPTR LIVSNFSQAK |
| | 1570 1580 1590 1600 1610 1620 |
| | QKSHLVDPEI LRDQSRLAPE IITATQYKKC DEFQTGILIY EMLHLPNPFD ENPELKEREY |
| | 1630 1640 1650 1660 1670 1680 |
| | TRADLPRIPF RSPYSRGLQQ LASCLLNPNP SERILISDAK GILQCLLWGP REDLFQTFTA |
| | 1690 1700 1710 1720 1730 1740 |
| | CPSLVQRNTL LQNWLDIKRT LLMIKFAEKS LDREGGISLE DWLCAQYLAF ATTDSLSCIV |
| | KILQHR |
| Q911497 | 10 20 30 40 50 60 |
| SEQ ID NO: 11 | MLRGPWRQLW LFFLLLLPGA PEPRGASRPW EGTDEPGSAW AWPGFQRLQE QLRAAGALSK |
| | 70 80 90 100 110 120 |
| | RYWTLFSCQV WPDDCDEDEE AATGPLGWRL PLLGQRYLDL LTTWYCSFKD CCPRGDCRIS |
| | 130 140 150 160 170 180 |
| | NNFTGLEWDL NVRLHGQHLV QQLVLRTVRG YLETPQPEKA LALSFHGWSG TGKNFVARML |
| | 190 200 210 220 230 240 |
| | VENLYRDGLM SDCVRMFIAT FHFPHPKYVD LYKEQLMSQI RETQQLCHQT LFIFDEAEKL |
| | 250 260 270 280 290 300 |
| | HPGLLEVLGP HLERRAPEGH RAESPWTIFL FLSNLRGDII NEVVLKLLKA GWSREEITME |
| | 310 320 330 340 350 360 |
| | HLEPHLQAEI VETIDNGFGH SRLVKENLID YFIPFLPLEY RHVRLCARDA FLSQELLYKE |
| | 370 380 390 |
| | ETLDEIAQMM VYVPKEEQLF SSQGCKSISQ RINYFLS |
| Q9UE35 | 10 20 30 40 50 60 |
| SEQ ID NO: 12 | MTMTLHTKAS GMALLHQIQG NELEPLNRPQ LKIPLERPLG EVYLDSSKPA VYNYPEGAAY |
| | 70 80 90 100 110 |
| | EFNAAAAANA QVYGQTGLPY GPGSEAAAFG SNGLGGFPPL NSVSPSPLML LHPPP |
| O00743 | 10 20 30 40 50 60 |
| SEQ ID NO: 13 | MAPLDLDKYV EIARLCKYLP ENDLKRLCDY VCDLLLEESN VQPVSTPVTV CGDIHGQFYD |
| | 70 80 90 100 110 120 |
| | LCELFRTGGQ VPDTNYIFMG DFVDRGYYSL ETFTYLLALK AKWPDRITLL RGNHESRQIT |
| | 130 140 150 160 170 180 |
| | QVYGFYDECQ TKYGNANAWR YCTKVFDMLT VAALIDEQIL CVHGGLSPDI KTLDQIRTIE |
| | 190 200 210 220 230 240 |
| | RNQEIPHKGA FCDLVWSDPE DVDTWAISPR GAGWLFGAKV TNEFVHINNL KLICRAHQLV |
| | 250 260 270 280 290 300 |
| | HEGYKFMFDE KLVTVWSAPN YCYRCGNIAS IMVFKDVNTR EPKLFRAVPD SERVIPPRTT |
| | TPYFL |
| Q8WXF8 | 10 20 30 40 50 60 |
| SEQ ID NO: 14 | MALSGSTPAP CWEEDECLDY YGMLSLHRMF EVVGGQLTEC ELELLAFLLD EAPGAAGGLA |
| | 70 80 90 100 110 120 |
| | RARSGLELLL ELERRGQCDE SNLRLLGQLL RVLARHDLLP HLARKRRRPV SPERYSYGTS |
| | 130 140 150 160 170 180 |
| | SSSKRTEGSC RRRRQSSSSA NSQQGQWETG SPPTKRQRRS RGRPSGGARR RRRGAPAAPQ |
| | 190 200 210 220 230 240 |
| | QQSEPARPSS EGKVTCDIRL RVRAEYCEHG PALEQGVASR RPQALARQLD VFGQATAVLR |
| | 250 260 270 280 290 300 |
| | SRDLGSVVCD IKFSELSYLD AFWGDYLSGA LLQALRGVFL TEALREAVGR EAVRLLVSVD |
| | 310 320 |
| | EADYEAGRRR LLLMEEEGGR RPTEAS |
| P81274 | 10 20 30 40 50 60 |
| SEQ ID NO: 15 | MEENLISMRE DHSFHVRYRM EASCLELALE GERLCKSGDC RAGVSFFEAA VQVGTEDLKT |
| | 70 80 90 100 110 120 |
| | LSAIYSQLGN AYFYLHDYAK ALEYHHHDLT LARTIGDQLG EAKASGNLGN TLKVLGNFDE |
| | 130 140 150 160 170 180 |
| | AIVCCQRHLD ISRELNDKVG EARALYNLGN VYHAKGKSFG CPGPQDVGEF PEEVRDALQA |
| | 190 200 210 220 230 240 |
| | AVDFYEENLS LVTALGDRAA QGRAFGNLGN THYLLGNFRD AVIAHEQRLL IAKEFGDKAA |
| | 250 260 270 280 290 300 |
| | ERRAYSNLGN AYIFLGEFET ASEYYKKTLL LARQLKDRAV EAQSCYSLGN TYTLLQDYEK |
| | 310 320 330 340 350 360 |
| | AIDYHLKHLA IAQELNDRIG EGRACWSLGN AYTALGNHDQ AMHFAEKHLE ISREVGDKSG |
| | 370 380 390 400 410 420 |
| | ELTARLNLSD LQMVLGLSYS TNNSIMSENT EIDSSLNGVR PKLGRRHSME NMELMKLTPE |
| | 430 440 450 460 470 480 |
| | KVQNWNSEIL AKQKPLIAKP SAKLLFVNRL KGKKYKTNSS TKVLQDASNS IDHRIPNSQR |
| | 490 500 510 520 530 540 |
| | KISADTIGDE GFFDLLSRFQ SNRMDDQRCC LQEKNCHTAS TTTSSTPPKM MLKTSSVPVV |
| | 550 560 570 580 590 600 |
| | SPNTDEFLDL LASSQSRRLD DQRASFSNLP GLRLTQNSQS VLSHLMTNDN KEADEDFFDI |
| | 610 620 630 640 650 660 |
| | LVKCQGSRLD DQRCAPPPAT TKGPTVPDED FFSLILRSQG KRMDEQRVLL QRDQNRDTDF |
| | 670 680 |
| | GLKDFLQNNA LLEFKNSGKK SADH |
| Q8NG08 | |
| SEQ ID NO: 16 | |
| | 10 20 30 40 50 60 |
| | MARSSPYLRQ LQGPLLPPRD LVEEDDDYLN DDVEEDEESV FIDAEELCSG GVKAGSLPGC |
| | 70 80 90 100 110 120 |
| | LRVSICDENT QETCKVFGRF PITGAWWRVK VQVKPVVGSR SYQYQVQGFP SYFLQSDMSP |
| | 130 140 150 160 170 180 |
| | PNQKHICALF LKECEVSSDD VNKFLTWVKE VSNYKNLNFE NLRETLRTFH KETGRKDQKQ |
| | 190 200 210 220 230 240 |
| | PTQNGQEELF LDNEMSLPLE NTIPFRNVMT ALQFPKIMEF LPVLLPRHFK WIIGSGSKEM |
| | 250 260 270 280 290 300 |
| | LKEIEEILGT HPWKLGFSKI TYREWKLLRC EASWIAFCQC ESLLQLMTDL EKNALIMYSR |
| | 310 320 330 340 350 360 |
| | LKQICREDGH TYVEVNDLTL TLSNHMSFHA ASESLKFLKD IGVVTYEKSC VFPYDLYHAE |
| | 370 380 390 400 410 420 |
| | RAIAFSICDL MKKPPWHLCV DVEKVLASIH TTKPENSSDD ALNESKPDEV RLENPVDVVD |
| | 430 440 450 460 470 480 |
| | TQDNGDHIWT NGENEINAEI SEVQLDQDQV EVPLDRDQVA ALEMICSNPV TVISGKGGCG |
| | 490 500 510 520 530 540 |
| | KTTIVSRLFK HIEQLEEREV KKACEDFEQD QNASEEWITF TEQSQLEADK AIEVLLTAPT |
| | 550 560 570 580 590 600 |
| | GKAAGLLRQK TGLHAYTLCQ VNYSFYSWTQ TMMTTNKPWK FSSVRVLVVD EGSLVSVGIF |
| | 610 620 630 640 650 660 |
| | KSVLNLLCEH SKLSKLIILG DIRQLPSIEP GNLLKDLFET LKSRNCAIEL KTNHRAESQL |
| | 670 680 690 700 710 720 |
| | IVDNATRISR RQFPKFDAEL NISDNPTLPI SIQDKTFIFV RLPEEDASSQ SSKTNHHSCL |
| | 730 740 750 760 770 780 |
| | YSAVKTLLQE NNLQNAKTSQ FIAFRRQDCD LINDCCCKHY TGHLTKDHQS RLVFGIGDKI |
| | 790 800 810 820 830 840 |
| | CCTRNAYLSD LLPENISGSQ QNNDLDASSE DFSGTLPDFA KNKRDFESNV RLCNGEIFFI |
| | 850 860 870 880 890 900 |
| | TNDVTDVTFG KRRSLTINNM AGLEVTVDFK KLMKYCRIKH AWARTIHTFQ GSEEQTVVYV |
| | 910 920 930 940 950 960 |
| | VGKAGRQHWQ HVYTAVTRGR CRVYVIAEES QLRNAIMKNS FPRKTRLKHF LQSKLSSSGA |
| | 970 980 990 1000 1010 1020 |
| | PPADFPSPRK SSGDSGGPST PSASPLPVVT DHAMTNDVTW SEASSPDERT LTFAERWQLS |
| | 1030 1040 1050 1060 107 1080 |
| | SPDGVDTDDD LPKSRASKRT CGVNDDESPS KIFMVGESPQ VSSRLQNLRL NNLIPRQLFK |
| | PTDNQET |
| Q96AE7 | 10 20 30 40 50 60 |
| SEQ ID NO: 17 | MAAAVGVRGR YELPPCSGPG WLLSLSALLS VAARGAFATT HWVVTEDGKI QQQVDSPMNL |
| | 70 80 90 100 110 120 |
| | KHPHDLVILM RQEATVNYLK ELEKQLVAQK IHIEENEDRD TGLEQRHNKE DPDCIKAKVP |
| | 130 140 150 160 170 180 |
| | LGDLDLYDGT YITLESKDIS PEDYIDTESP VPPDPEQPDC TKILELPYSI HAFQHLRGVQ |
| | 190 200 210 220 230 240 |
| | ERVNLSAPLL PKEDPIFTYL SKRLGRSIDD IGHLIHEGLQ KNTSSWVLYN MASFYWRIKN |
| | 250 260 270 280 290 300 |
| | EPYQVVECAM RALHFSSRHN KDIALVNLAN VLHRAHFSAD AAVVVHAALD DSDFFTSYYT |
| | 310 320 330 340 350 360 |
| | LGNIYAMLGE YNHSVLCYDH ALQARPGFEQ AIKRKHAVLC QQKLEQKLEA QHRSLQRTLN |
| | 370 380 390 400 410 420 |
| | ELKEYQKQHD HYLRQQEILE KHKLIQEEQI LRNIIHETQM AKEAQLGNHQ ICRLVNQQHS |
| | 430 440 450 460 470 480 |
| | LHCQWDQPVR YHRGDIFENV DYVQFGEDSS TSSMMSVNFD VQSNQSDIND SVKSSPVAHS |
| | 490 500 510 520 530 540 |
| | ILWIWGRDSD AYRDKQHILW PKRADCTESY PRVPVGGELP TYFLPPENKG LRIHELSSDD |
| | 550 560 570 580 590 600 |
| | YSTEEEAQTP DCSITDFRKS HTLSYLVKEL EVRMDLKAKM PDDHARKILL SRINNYTIPE |
| | 610 620 630 640 650 660 |
| | EEIGSFLFHA INKPNAPIWL ILNEAGLYWR AVGNSTFAIA CLQRALNLAP LQYQDVPLVN |
| | 670 680 690 700 710 720 |
| | LANLLIHYGL HLDATKLLLQ ALAINSSEPL TFLSLGNAYL ALKNISGALE AFRQALKLTT |
| | 730 740 750 760 770 780 |
| | KCPECENSLK LIRCMQFYPF LYNITSSVCS GTVVEESNGS DEMENSDETK MSEEILALVD |
| | 790 800 810 820 830 840 |
| | EFQQAWPLEG FGGALEMKGR RLDLQGIRVL KKGPQDGVAR SSCYGDCRSE DDEATEWITF |
| | 850 860 870 880 890 900 |
| | QVKRVKKPKG DHKKTPGKKV ETGQIENGHR YQANLEITGP KVASPGPQGK KRDYQRLGWP |
| | 910 920 930 940 950 960 |
| | SPDECLKLRW VELTAIVSTW LAVSSKNIDI TEHIDFATPI QQPAMEPLCN GNLPTSMHTL |
| | 970 980 990 1000 1010 1020 |
| | DHLHGVSNRA SLHYTGESQL TEVLQNLGKD QYPQQSLEQI GTRIAKVLEK NQTSWVLSSM |
| | 1030 1040 1050 1060 1070 1080 |
| | AALYWRVKGQ GKKAIDCLRQ ALHYAPHQMK DVPLISLANI LHNAKLWNDA VIVATMAVEI |
| | 1090 1100 1110 1120 1130 1140 |
| | APHFAVNHFT LGNVYVAMEE FEKALVWYES TLKLQPEFVP AKNRIQTIQC HLMLKKGRRS |
| | P |
| Q9BZM4 | 10 20 30 40 50 60 |
| SEQ ID NO: 18 | MAAAASPAIL PRLAILPYLL FDWSGTGRAD AHSLWYNFTI IHLPRHGQQW CEVQSQVDQK |
| | 70 80 90 100 110 120 |
| | NFLSYDCGSD KVLSMGHLEE QLYATDAWGK QLEMLREVGQ RLRLELADTE LEDFTPSGPL |
| | 130 140 150 160 170 180 |
| | TLQVRMSCEC EADGYIRGSW QFSFDGRKFL LFDSNNRKWT VVHAGARRMK EKWEKDSGLT |
| | 190 200 210 220 230 240 |
| | TFFKMVSMRD CKSWLRDFLM HRKKRLEPTA PPTMAPGLAQ PKAIATTLSP WSFLIILCFI |
| | LPGI |
| Q5T2D3 | 10 20 30 40 50 60 |
| SEQ ID NO: 19 | MSRKQAAKSR PGSGSRKAEA ERKRDERAAR RALAKERRNR PESGGGGGCE EEFVSFANQL |
| | 70 80 90 100 110 120 |
| | QALGLKLREV PGDGNCLFRA LGDQLEGHSR NHLKHRQETV DYMIKQREDF EPFVEDDIPF |
| | 130 140 150 160 170 180 |
| | EKHVASLAKP GTFAGNDAIV AFARNHQLNV VIHQLNAPLW QIRGTEKSSV RELHIAYRYG |
| | 190 200 210 220 230 240 |
| | EHYDSVRRIN DNSEAPAHLQ TDFQMLHQDE SNKREKIKTK GMDSEDDLRD EVEDAVQKVC |
| | 250 260 270 280 290 300 |
| | NATGCSDFNL IVQNLEAENY NIESAIIAVL RMNQGKRNNA EENLEPSGRV LKQCGPLWEE |
| | 310 320 330 340 350 360 |
| | GGSGARIFGN QGLNEGRTEN NKAQASPSEE NKANKNQLAK VTNKQRREQQ WMEKKKRQEE |
| | 370 380 390 |
| | RHRHKALESR GSHRDNNRSE AEANTQVTLV KTFAALNI |
| Q8IXT5 | 10 20 30 40 50 60 |
| SEQ ID NO: 20 | MAVVIRLLGL PFIAGPVDIR HFFTGLTIPD GGVHIIGGEI GEAFIIFATD EDARRAISRS |
| | 70 80 90 100 110 120 |
| | GGFIKDSSVE LFLSSKAEMQ KTIEMKRTDR VGRGRPGSGT SGVDSLSNFI ESVKEEASNS |
| | 130 140 150 160 170 180 |
| | GYGSSINQDA GFHTNGTGHG NLRPRKTRPL KAENPYLFLR GLPYLVNEDD VRVFFSGLCV |
| | 190 200 210 220 230 240 |
| | DGVIFLKHHD GRNNGDAIVK FASCVDASGG LKCHRSFMGS RFIEVMQGSE QQWIEFGGNA |
| | 250 260 270 280 290 300 |
| | VKEGDVLRRS EEHSPPRGIN DRHFRKRSHS KSPRRTRSRS PLGFYVHLKN LSLSIDERDL |
| | 310 320 330 340 350 360 |
| | RNFFRGTDLT DEQIRFLYKD ENRTRYAFVM FKTLKDYNTA LSLHKTVLQY RPVHIDPISR |
| | 370 380 390 400 410 420 |
| | KQMLKFIARY EKKRSGSLER DRPGHVSQKY SQEGNSGQKL CIYIRNFPFD VTKVEVQKFF |
| | 430 440 450 460 470 480 |
| | ADFLLAEDDI YLLYDDKGVG LGEALVKFKS EEQAMKAERL NRRRFLGTEV LLRLISEAQI |
| | 490 500 510 520 530 540 |
| | QEFGVNFSVM SSEKMQARSQ SRERGDHSHL FDSKDPPIYS VGAFENFRHQ LEDLRQLDNF |
| | 550 560 570 580 590 600 |
| | KHPQRDFRQP DRHPPEDFRH SSEDFRFPPE DFRHSPEDFR RPREEDFRRP SEEDFRRPWE |
| | 610 620 630 640 650 660 |
| | EDFRRPPEDD FRHPREEDWR RPLEEDWRRP LEEDFRRSPT EDFRQLPEED FRQPPEEDLR |
| | 670 680 690 700 710 720 |
| | WLPEEDFRRP PEEDWRRPPE EDFRRPLQGE WRRPPEDDFR RPPEEDFRHS PEEDFRQSPQ |
| | 730 740 750 760 770 780 |
| | EHFRRPPQEH FRRPPPEHFR RPPPEHFRRP PPEHFRRPPP EHFRRPPPEH FRRPPPEHFR |
| | 790 800 810 820 830 840 |
| | RPPQEHFRRP PQEHFRRSRE EDFRHPPDED FRGPPDEDFR HPPDEDFRSP QEEDFRCPSD |
| | 850 860 870 880 890 900 |
| | EDFRQLPEED LREAPEEDPR LPDNFRPPGE DFRSPPDDFR SHRPFVNFGR PEGGKFDFGK |
| | 910 920 930 940 950 960 |
| | HNMGSFPEGR FMPDPKINCG SGRVTPIKIM NLPFKANVNE ILDFFHGYRI IPDSVSIQYN |
| | 970 980 990 1000 |
| | EQGLPTGEAI VAMINYNEAM AAIKDLNDRP VGPRKVKLTL L |
| Q9P225 | 10 20 30 40 50 60 |
| SEQ ID NO: 21 | MSSKAEKKQR LSGRGSSQAS WSGRATRAAV ATQEQGNAPA VSEPELQAEL PKEEPEPRLE |
| | 70 80 90 100 110 120 |
| | GPQAQSEESV EPEADVKPLF LSRAALTGLA DAVWTQEHDA ILEHFAQDPT ESILTIFIDP |
| | 130 140 150 160 170 180 |
| | CFGLKLELGM PVQTQNQLVY FIRQAPVPIT WENFEATVQF GTVRGPYIPA LLRLLGGVFA |
| | 190 200 210 220 230 240 |
| | PQIFANTGWP ESIRNHFASH LHKFLACLTD TRYKLEGHTV LYIPAEAMNM KPEMVIKDKE |
| | 250 260 270 280 290 300 |
| | LVQRLETSMI HWTRQIKEML SAQETVETGE NLGPLEEIEF WRNRCMDLSG ISKQLVKKGV |
| | 310 320 330 340 350 360 |
| | KHVESILHLA KSSYLAPFMK LAQQIQDGSR QAQSNLTFLS ILKEPYQELA FMKPKDISSK |
| | 370 380 390 400 410 420 |
| | LPKLISLIRI IWVNSPHYNT RERLTSLFRK VCDCQYHFAR WEDGKQGPLP CFFGAQGPQI |
| | 430 440 450 460 470 480 |
| | TRNLLEIEDI FHKNLHTLRA VRGGILDVKN TCWHEDYNKF RAGIKDLEVM TQNLITSAFE |
| | 490 500 510 520 530 540 |
| | LVRDVPHGVL LLDTFHRLAS REAIKRTYDK KAVDLYMLFN SELALVNRER NKKWPDLEPY |
| | 550 560 570 580 590 600 |
| | VAQYSGKARW VHILRRRIDR VMTCLAGAHF LPRIGTGKES VHTYQQMVQA IDELVRKTFQ |
| | 610 620 630 640 650 660 |
| | EWTSSLDKDC IRRLDTPLLR ISQEKAGMLD VNFDKSLLIL FAEIDYWERL LFETPHYVVN |
| | 670 680 690 700 710 720 |
| | VAERAEDLRI LRENLLLVAR DYNRIIAMLS PDEQALFKER IRLLDKKIHP GLKKLHWALK |
| | 730 740 750 760 770 780 |
| | GASAFFITEC RIHASKVQMI VNEFKASTLT IGWRAQEMSE KLLVRISGKR VYRDLEFEED |
| | 790 800 810 820 830 840 |
| | QREHRAAVQQ KLMNLHQDVV TIMTNSYEVF KNDGPEIQQQ WMLYMIRLDR MMEDALRLNV |
| | 850 860 870 880 890 900 |
| | KWSLLELSKA INGDGKTSPN PLFQVLVILK NDLQGSVAQV EFSPTLQTLA GVVNDIGNHL |
| | 910 920 930 940 950 960 |
| | FSTISVFCHL PDILTKRKLH REPIQTVVEQ DEDIKKIQTQ ISSGMTNNAS LLQNYLKTWD |
| | 970 980 990 1000 1010 1020 |
| | MYREIWEINK DSFIHRYQRL NPPVSSFVAD IARYTEVANN VQKEETVTNI QFVLLDCSHL |
| | 1030 1040 1050 1060 1070 1080 |
| | KFSLVQHCNE WQNKFATLLR EMAAGRLLEL HTYLKENAEK ISRPPQTLEE LGVSLQLVDA |
| | 1090 1100 1110 1120 1130 1140 |
| | LKHDLANVET QIPPIHEQFA ILEKYEVPVE DSVLEMLDSL NGEWVVFQQT LLDSKQMLKK |
| | 1150 1160 1170 1180 1190 1200 |
| | HKEKFKTGLI HSADDFKKKA HTLLEDFEFK GHFTSNVGYM SALDQITQVR AMLMAMREEE |
| | 1210 1220 1230 1240 1250 1260 |
| | NSLRANLGIF KIEQPPSKDL QNLEKELDAL QQIWEIARDW EENWNEWKTG RFLILQTETM |
| | 1270 1280 1290 1300 1310 1320 |
| | ETTAHGLFRR LTKLAKEYKD RNWEIIETTR SKIEQFKRTM PLISDLRNPA LRERHWDQVR |
| | 1330 1340 1350 1360 1370 1380 |
| | DEIQREFDQE SESFTLEQIV ELGMDQHVEK IGEISASATK ELAIEVALQN IAKTWDVTQL |
| | 1390 1400 1410 1420 1430 1440 |
| | DIVPYKDKGH HRLRGTEEVF QALEDNQVAL STMKASRFVK AFEKDVDHWE RCLSLILEVI |
| | 1450 1460 1470 1480 1490 1500 |
| | EMILTVQRQW MYLENIFLGE DIRKQLPNES TLFDQVNSNW KAIMDRMNKD NNALRSTHHP |
| | 1510 1520 1530 1540 1550 1560 |
| | GLLDTLIEMN TILEDIQKSL DMYLETKRHI FPRFYFLSND DLLEILGQSR NPEAVQPHLK |
| | 1570 1580 1590 1600 1610 1620 |
| | KCFDNIKLLR IQKVGGPSSK WEAVGMFSGD GEYIDFLHSV FLEGPVESWL GDVEQTMRVT |
| | 1630 1640 1650 1660 1670 1680 |
| | LRDLLRNCHL ALRKFLNKRD KWVKEWAGQV VITASQIQWT ADVTKCLLTA KERADKKILK |
| | 1690 1700 1710 1720 1730 1740 |
| | VMKKNQVSIL NKYSEAIRGN LTKIMRLKIV ALVTIEIHAR DVLEKLYKSG LMDVNSFDWL |
| | 1750 1760 1770 1780 1790 1800 |
| | SQLRFYWEKD LDDCVIRQTN TQFQYNYEYL GNSGRLVITP LTDRCYMTLT TALHLHRGGS |
| | 1810 1820 1830 1840 1850 1860 |
| | PKGPAGTGKT ETVKDLGKAL GIYVIVVNCS EGLDYKSMGR MYSGLAQTGA WGCFDEFNRI |
| | 1870 1880 1890 1900 1910 1920 |
| | NIEVLSVVAH QILCILSALA AGLTHFHFDG FEINLVWSCG IFITMNPGYA GRTELPENLK |
| | 1930 1940 1950 1960 1970 1980 |
| | SMFRPIAMVV PDSTLIAEII LFGEGFGNCK ILAKKVYTLY SLAVQQLSRQ DHYDFGLRAL |
| | 1990 2000 2010 2020 2030 2040 |
| | TSLLRYAGKK RRLQPDLTDE EVLLLSMRDM NIAKLTSVDA PLFNAIVQDL FPNIELPVID |
| | 2050 2060 2070 2080 2090 2100 |
| | YGKLRETVEQ EIRDMGLQST PFTLTKVFQL YETKNSRHST MIVGCTGSGK TASWRILQAS |
| | 2110 2120 2130 2140 2150 2160 |
| | LSSLCRAGDP NFNIVREFPL NPKALSLGEL YGEYDLSTNE WTDGILSSVM RTACADEKPD |
| | 2170 2180 2190 2200 2210 2220 |
| | EKWILFDGPV DTLWIENMNS VMDDNKVLTL INGERIAMPE QVSLLFEVED LAMASPATVS |
| | 2230 2240 2250 2260 2270 2280 |
| | RCGMVYTDYA DLGWKPYVQS WLEKRPKAEV EPLQRMFEKL INKMLAFKKD NCKELVPLPE |
| | 2290 2300 2310 2320 2330 2340 |
| | YSGITSLCKL YSALATPENG VNPADGENYV TMVEMTFVFS MIWSVCASVD EEGRKRIDSY |
| | 2350 2360 2370 2380 2390 2400 |
| | LREIEGSFPN KDTVYEYFVD PKIRSWTSFE DKLPKSWRYP PNAPFYKIMV PTVDTVRYNY |
| | 2410 2420 2430 2440 2450 2460 |
| | LVSSLVANQN PILLVGPVGT GKTSIAQSVL QSLPSSQWSV LVVNMSAQTT SNNVQSIIES |
| | 2470 2480 2490 2500 2510 2520 |
| | RVEKRTKGVY VPFGGKSMIT FMDDLNMPAK DMFGSQPPLE LIRLWIDYGF WYDRTKQTIK |
| | 2530 2540 2550 2560 2570 2580 |
| | YIREMFLMAA MGPPGGGRTV ISPRLRSRFN IINMTFPTKS QIIRIFGTMI NQKLQDFEEE |
| | 2590 2600 2610 2620 2630 2640 |
| | VKPIGNVVTE ATLDMYNTVV QRFLPTPTKM HYLFNLRDIS KVFQGMLRAN KDFHDTKSSI |
| | 2650 2660 2670 2680 2690 2700 |
| | TRLWIHECFR VFSDRLVDAA DTEAFMGIIS DKLGSFFDLT FHHLCPSKRP PIFGDFLKEP |
| | 2710 2720 2730 2740 2750 2760 |
| | KVYEDLTDLT VLKTVMETAL NEYNLSPSVV PMQLVLFREA IEHITRIVRV IGQPRGNMLL |
| | 2770 2780 2790 2800 2810 2820 |
| | VGIGGSGRQS LARLASSICD YTTFQIEVTK HYRKQEFRDD IKRLYRQAGV ELKTTSFIFV |
| | 2830 2840 2850 2860 2870 2880 |
| | DTQIADESFL EDINNILSSG EVPNLYKPDE FEEIQSHIID QARVEQVPES SDSLFAYLIE |
| | 2890 2900 2910 2920 2930 2940 |
| | RVQNNLHIVL CLSPMGDPFR NWIRQYPALV NCTTINWFSE WPQEALLEVA EKCLIGVDLG |
| | 2950 2960 2970 2980 2990 3000 |
| | TQENIHRKVA QIFVTMHWSV AQYSQKMLLE LRRHNYVTPT KYLELLSGYK KLLGEKRQEL |
| | 3010 3020 3030 3040 3050 3060 |
| | LAQANKLRTG LFKIDETREK VQVMSLELED AKKKVAEFQK QCEEYLVIIV QQKREADEQQ |
| | 3070 3080 3090 3100 3110 3120 |
| | KAVTANSEKI AVEEIKCQAL ADNAQKDLEE ALPALEEAMR ALESLNKKDI GEIKSYGRPP |
| | 3130 3140 3150 3160 3170 3180 |
| | AQVEIVMQAV MILRGNEPTW AEAKRQLGEQ NFIKSLINFD KDNISDKVLK KIGAYCAQPD |
| | 3190 3200 3210 3220 3230 3240 |
| | FQPDIIGRVS LAAKSLCMWV RAMELYGRLY RVVEPKRIRM NAALAQLREK QAALAEAQEK |
| | 3250 3260 3270 3280 3290 3300 |
| | LREVAEKLEM LKKQYDEKLA QKEELRKKSE EMELKLERAG MLVSGLAGEK ARWEETVQGL |
| | 3310 3320 3330 3340 3350 3360 |
| | EEDLGYLVGD CLLAAAFLSY MGPFLTNYRD EIVNQIWIGK IWELQVPCSP SFAIDNFLCN |
| | 3370 3380 3390 3400 3410 3420 |
| | PTKVRDWNIQ GLPSDAFSTE NGIIVTRGNR WALMIDPQAQ ALKWIKNMEG GQGLKIIDLQ |
| | 3430 3440 3450 3460 3470 3480 |
| | MSDYLRILEH AIHFGYPVLL QNVQEYLDPT LNPMLNKSVA RIGGRLLMRI GDKEVEYNTN |
| | 3490 3500 3510 3520 3530 3540 |
| | FRFYITTKLS NPHYSPETSA KTTIVNFAVK EQGLEAQLLG IVVRKERPEL EEQKDSLVIN |
| | 3550 3560 3570 3580 3590 3600 |
| | IAAGKRKLKE LEDEILRLLN EATGSLLDDV QLVNTLHTSK ITATEVTEQL ETSETTEINT |
| | 3610 3620 3630 3640 3650 3660 |
| | DLAREAYRPC AQRASILFFV LNDMGCIDPM YQFSLDAYIS LFILSIDKSH RSNKLEDRID |
| | 3670 3680 3690 3700 3710 3720 |
| | YLNDYHTYAV YRYTCRTLFE RHKLLFSFHM CAKILETSGK LNMDEYNFFL RGGVVLDREG |
| | 3730 3740 3750 3760 3770 3780 |
| | QMDNPCSSWL ADAYWDNITE LDKLTNFHGL MNSFEQYPRD WHLWYTNAAP EKAMLPGEWE |
| | 3790 3800 3810 3820 3830 3840 |
| | NACNEMQRML IVRSLRQDRV AFCVTSFIIT NLGSRFIEPP VLNMKSVLED STPRSPLVFI |
| | 3850 3860 3870 3880 3890 3900 |
| | LSPGVDPTSA LLQLAEHMGM AQRFHALSLG QGQAPIAARL LREGVTQGHW VFLANCHLSL |
| | 3910 3920 3930 3940 3950 3960 |
| | SWMPNLDKLV EQLQVEDPHP SFRLWLSSIP HPDFPISILQ VSIKMTTEPP KGLKANMTRL |
| | 3970 3980 3990 4000 4010 4020 |
| | YQLMSEPQFS RCSKPAKYKK LLFSLCFFHS VLLERKKFLQ LGWNIIYGFN DSDFEVSENL |
| | 4030 4040 4050 4060 4070 4080 |
| | LSLYLDEYEE TPWDALKYLI AGINYGGHVT DDWDRRLLTT YINDYFCDQS LSTPFHRLSA |
| | 4090 4100 4110 4120 4130 4140 |
| | LETYFIPKDG SLASYKEYIS LLPGMDPPEA FGQHPNADVA SQITEAQTLF DTLLSLQPQI |
| | 4150 4160 4170 4180 4190 4200 |
| | TPTRAGGQTR EEKVLELAAD VKQKIPEMID YEGTQKLLAL DPSPLNVVLL QEIQRYNTLM |
| | 4210 4220 4230 4240 4250 4260 |
| | QTILFSLTDL EKGIQGLIVM STSLEEIFNC IFDAHVPPLW GKAYPSQKPL AAWTRDLAMR |
| | 4270 4280 4290 4300 4310 4320 |
| | VEQFELWASR ARPPVIFWLS GFTFPTGFLT AVLQSSARQN NVSVDSLSWE FIVSTVDDSN |
| | 4330 4340 4350 4360 4370 4380 |
| | LVYPPKDGVW VRGLYLEGAG WDRKNSCLVE AEPMQLVCLM PTIHFRPAES RKKSAKGMYS |
| | 4390 4400 4410 4420 |
| | CPCYYYPNRA GSSDRASFVI GIDLRSGAMT PDHWIKRGTA LLMSLDS |
| Q9Y2I9 | 10 20 30 40 50 60 |
| SEQ ID NO: 22 | MDVLPTGGGR PGLRTELEFR GGGGEARLES QEEETIPAAP PAPRLRGAAE RPRRSRDTWD |
| | 70 80 90 100 110 120 |
| | GDEDTEPGEA CGGRTSRTAS LVSGLLNELY SCTEEEEAAG GGRGAEGRRR RRDSLDSSTE |
| | 130 140 150 160 170 180 |
| | ASGSDVVLGG RSGAGDSRVL QELQERPSQR HQMLYLRQKD ANELKTILRE LKYRIGIQSA |
| | 190 200 210 220 230 240 |
| | KLLRHLKQKD RLLHKVQRNC DIVTACLQAV SQKRRVDTKL KFTLEPSLGQ NGFQQWYDAL |
| | 250 260 270 280 290 300 |
| | KAVARLSTGI PKEWRRKVWL TLADHYLHSI AIDWDKTMRF TFNERSNPDD DSMGIQIVKD |
| | 310 320 330 340 350 360 |
| | LHRTGCSSYC GQEAEQDRVV LKRVLLAYAR WNKTVGYCQG FNILAALILE VMEGNEGDAL |
| | 370 380 390 400 410 420 |
| | KIMIYLIDKV LPESYFVNNL RALSVDMAVF RDLLRMKLPE LSQHLDTLQR TANKESGGGY |
| | 430 440 450 460 470 480 |
| | EPPLTNVFTM QWFLTLFATC LPNQTVLKIW DSVFFEGSEI ILRVSLAIWA KLGEQIECCE |
| | 490 500 510 520 530 540 |
| | TADEFYSTMG RLTQEMLEND LLQSHELMQT VYSMAPFPFP QLAELREKYT YNITPFPATV |
| | 550 560 570 580 590 600 |
| | KPTSVSGRHS KARDSDEEND PDDEDAVVNA VGCLGPFSGF LAPELQKYQK QIKEPNEEQS |
| | 610 620 630 640 650 660 |
| | LRSNNIAELS PGAINSCRSE YHAAFNSMMM ERMTTDINAL KRQYSRIKKK QQQQVHQVYI |
| | 670 680 690 700 710 720 |
| | RADKGPVTSI LPSQVNSSPV INHLLLGKKM KMTNRAAKNA VIHIPGHTGG KISPVPYEDL |
| | 730 740 750 760 770 780 |
| | KTKLNSPWRT HIRVHKKNMP RTKSHPGCGD TVGLIDEQNE ASKTNGLGAA EAFPSGCTAT |
| | 790 800 810 820 830 840 |
| | AGREGSSPEG STRRTIEGQS PEPVFGDADV DVSAVQAKLG ALELNQRDAA AETELRVHPP |
| | 850 860 870 880 890 900 |
| | CQRHCPEPPS APEENKATSK APQGSNSKTP IFSPFPSVKP LRKSATARNL GLYGPTERTP |
| | 910 920 |
| | TVHFPQMSRS FSKPGGGNSG TKKR |

TABLE 2

| | |
|---|---|
| P41222 | 10 20 30 40 50 60 |
| (PTGDS) | MATHHTLWMG LALLGVLGDL QAAPEAQVSV QPNFQQDKFL GRWFSAGLAS NSSWLREKKA |
| SEQ ID NO: 23 | 70 80 90 100 110 120 |
| | ALSMCKSVVA PATDGGLNLT STFLRKNQCE TRTMLLQPAG SLGSYSYRSP HWGSTYSVSV |
| | 130 140 150 160 170 180 |
| | VETDYDQYAL LYSQGSKGPG EDFRMATLYS RTQTPRAELK EKFTAFCKAQ GFTEDTIVFL |
| | 190 |
| | PQTDKCMTEQ |
| P14151 | 10 20 30 40 50 60 |
| (SELL) | MIFPWKCQST QRDLWNIFKL WGWTMLCCDF LAHHGTDCWT YHYSEKPMNW QRARRFCRDN |
| SEQ ID NO: 24 | 70 80 90 100 110 120 |
| | YTDLVAIQNK AEIEYLEKTL PFSRSYYWIG IRKIGGIWTW VGTNKSLTEE AENWGDGEPN |
| | 130 140 150 160 170 180 |
| | NKKNKEDCVE IYIKRNKDAG KWNDDACHKL KAALCYTASC QPWSCSGHGE CVEIINNYTC |
| | 190 200 210 220 230 240 |
| | NCDVGYYGPQ CQFVIQCEPL EAPELGTMDC THPLGNFSFS SQCAFSCSEG TNLTGIEETT |
| | 250 260 270 280 290 300 |
| | CGPFGNWSSP EPTCQVIQCE PLSAPDLGIM NCSHPLASFS FTSACTFICS EGTELIGKKK |
| | 310 320 330 340 350 360 |
| | TICESSGIWS NPSPICQKLD KSFSMIKEGD YNPLFIPVAV MVTAFSGLAF IIWLARRLKK |
| | 370 |
| | GKKSKRSMND PY |
| Q06418 | 10 20 30 40 50 60 |
| (TYRO3) | TVEGTRANLT GWDPQKDLIV RVCVSNAVGC GPWSQPLVVS SHDRAGQQGP PHSRTSWVPV |
| SEQ ID NO: 25 | 70 80 90 100 110 120 |
| | VLGVLTALVT AAALALILLR KRRKETRFGQ AFDSVMARGE PAVHFRAARS FNRERPERIE |
| | 130 140 150 160 170 180 |
| | ATLDSLGISD ELKEKLEDVL IPEQQFTLGR MLGKGEFGSV REAQLKQEDG SFVKVAVKML |
| | 190 200 210 220 230 240 |
| | KADIIASSDI EEFLREAACM KEFDHPHVAK LVGVSLRSRA KGRLPIPMVI LPFMKHGDLH |
| | 250 260 270 280 290 300 |
| | AFLLASRIGE NPFNLPLQTL IRFMVDIACG MEYLSSRNFI HRDLAARNCM LAEDMTVCVA |
| | 310 320 330 340 350 360 |
| | DFGLSRKIYS GDYYRQGCAS KLPVKWLALE SLADNLYTVQ SDVWAFGVTM WEIMTRGQTP |
| | 370 380 390 400 410 420 |
| | YAGIENAEIY NYLIGGNRLK QPPECMEDVY DLMYQCWSAD PKQRPSFTCL RMELENILGQ |
| | 430 440 450 460 470 480 |
| | LSVLSASQDP LYINIERAEE PTAGGSLELP GRDQPYSGAG DGSGMGAVGG TPSDCRYILT |
| | 490 500 510 |
| | PGGLAEQPGQ AEHQPESPLN ETQRLLLLQQ GLLPHSSC |
| P52306 | 10 20 30 40 50 60 |
| (RAP1GDS1) | MDNLSDTLKK LKITAVDKTE DSLEGCLDCL LQALAQNNTE TSEKIQASGI LQLFASLLTP |
| SEQ ID NO: 26 | 70 80 90 100 110 120 |
| | QSSCKAKVAN IIAEVAKNEF MRIPCVDAGL ISPLVQLLNS KDQEVLLQTG RALGNICYDS |
| | 130 140 150 160 170 180 |
| | HEGRSAVDQA GGAQIVIDHL RSLCSITDPA NEKLLTVFCG MLMNYSNEND SLQAQLINMG |
| | 190 200 210 220 230 240 |
| | VIPTLVKLLG IHCQNAALTE MCLVAFGNLA ELESSKEQFA STNIAEELVK LFKKQIEHDK |
| | 250 260 270 280 290 300 |
| | REMIFEVLAP LAENDAIKLQ LVEAGLVECL LEIVQQKVDS DKEDDITELK TGSDLMVLLL |
| | 310 320 330 340 350 360 |
| | LGDESMQKLF EGGKGSVFQR VLSWIPSNNH QLQLAGALAI ANFARNDANC IHMVDNGIVE |
| | 370 380 390 400 410 420 |
| | KLMDLLDRHV EDGNVTVQHA ALSALRNLAI PVINKAKMLS AGVTEAVLKF LKSEMPPVQF |
| | 430 440 450 460 470 480 |
| | KLLGTLRMLI DAQAEAAEQL GKNVKLVERL VEWCEAKDHA GVMGESNRLL SALIRHSKSK |
| | 490 500 510 520 530 540 |
| | DVIKTIVQSG GIKHLVTMAT SEHVIMQNEA LVALALIAAL ELGTAEKDLE SAKLVQILHR |
| | 550 560 570 580 590 600 |
| | LLADERSAPE IKYNSMVLIC ALMGSECLHK EVQDLAFLDV VSKLRSHENK SVAQQASLTE |
| | QRLTVES |
| Q9Y5Y7 | 10 20 30 40 50 60 |
| (LYVE1) | MARCFSLVLL LTSIWTTRLL VQGSLRAEEL SIQVSCRIMG ITLVSKKANQ QLNFTEAKEA |
| SEQ ID NO: 27 | 70 80 90 100 110 120 |
| | CRLLGLSLAG KDQVETALKA SFETCSYGWV GDGFVVISRI SPNPKCGKNG VGVLIWKVPV |
| | 130 140 150 160 170 180 |
| | SRQFAAYCYN SSDTWTNSCI PEIITTKDPI FNTQTATQTT EFIVSDSTYS VASPYSTIPA |
| | 190 200 210 220 230 240 |
| | PTTTPPAPAS TSIPRRKKLI CVTEVFMETS TMSTETEPFV ENKAAFKNEA AGFGGVPTAL |
| | 250 260 270 280 290 300 |
| | LVLALLFFGA AAGLGFCYVK RYVKAFPFTN KNQQKEMIET KVVKEEKAND SNPNEESKKT |
| | 310 320 |
| | DKNPEESKSP SKTTVRCLEA EV |

1. A method of assessing or monitoring systemic HIV viral load in an HIV-infected human patient, the method comprising the steps of:
analyzing a test sample comprising urine from the patient for the presence or concentration of at least one protein, whereby a test data set is obtained; and,
comparing the test data set with a control data set relating to the presence or concentration of the at least one protein in a control sample;
whereby the HIV viral load in the patient is assessed or monitored.

2. The method of claim 1, wherein the patient has received or is receiving a first anti-HIV medication.

3. The method of claim 1, wherein the patient is a new-born human or an infant younger than about 18 months of age.

4. The method of claim 1, wherein the test sample is prepared by a method comprising subjecting urine from the patient to at least one procedure selected from the group consisting of protein isolation and protein digestion.

5. The method of claim 1, wherein the test sample is analyzed using mass spectrometry, a quantum dot assay or a chromophore assay.

6. The method of claim 1, wherein the test sample is analyzed using a method comprising contacting the test sample with an antibody or aptamer.

7. The method of claim 6, wherein the antibody is at least one selected from the group consisting of a polyclonal antibody, monoclonal antibody, Fv, Fab, F(ab)₂, single chain antibody, human antibody, humanized antibody, and fragments and derivatives thereof.

8. The method of claim 6, wherein the antibody or aptamer is used in an immunoassay.

9. The method of claim 8, wherein the immunoassay comprises at least one selected from the group consisting of immunoturbidimetry, immunonephelometry, ELISA assay, radioimmunoassay, chemiluminescence immunoassay, immunofluorescence, immunoprecipitation, immunoelectrophoresis, and flow cytometry-based immunoassay.

10. The method of claim 1, wherein the control sample comprises an urine sample from at least one selected from the group consisting of: an untreated HIV-infected control human, an HIV-uninfected control human, and an HIV-infected control human with controlled infection.

11. The method of claim 10, wherein the untreated HIV-infected control human is the human patient before receiving anti-HIV medication.

12. (canceled)

13. (canceled)

14. The method of claim 1, wherein the concentration of the protein in the patient's urine is higher by at least a multiplicity factor than the concentration of the protein in the urine from an HIV-uninfected control human or from an HIV-infected control human with controlled infection, wherein the patient is identified as having uncontrolled HIV infection, whereby the patient is prescribed a second anti-HIV medication that is distinct from the first anti-HIV medication.

15. The method of claim 14, wherein the multiplicity factor is selected from the group consisting of about 1.1, 1.2, 1.3, 1.4, 1.5, 1.6, 1.7. 1.8, 1.9, 2, 2.25, 2.5, 2.75, 3, 3.5, 4, 5, 6, 7, 8, 9, 10, 11, 12, 13, 14, 15, 20, 30, 40, 50, 75, 100, 125, 250, 500 and 1,000.

16. The method of claim 1, wherein the concentration of the protein in the patient's urine is lower by at least a multiplicity factor than the concentration of the protein in the urine from an HIV-uninfected control human or from an HIV-infected control human with controlled infection, wherein the patient is identified as having controlled HIV infection, whereby the patient continues to be prescribed the first anti-HIV medication.

17. The method of claim 16, wherein the multiplicity factor is selected from the group consisting of about 1.1, 1.2, 1.3, 1.4, 1.5, 1.6, 1.7. 1.8, 1.9, 2, 2.25, 2.5, 2.75, 3, 3.5, 4, 5, 6, 7, 8, 9, 10, 11, 12, 13, 14, 15, 20, 30, 40, 50, 75, 100, 125, 250, 500 and 1,000.

18. The method of claim 1, wherein the concentration of the protein in the patient's urine is equal to or greater than a multiplicity factor of the concentration of the protein in the urine from an untreated HIV-positive control human, wherein the patient is identified as having uncontrolled HIV infection, whereby the patient is prescribed a second anti-HIV medication which is distinct from the first anti-HIV medication.

19. The method of claim 18, wherein the multiplicity factor is selected from the group consisting of about 1, 0.95, 0.9, 0.8, 0.7, 0.6, 0.5, 0.4, 0.3, 0.2, 0.1, 0.05, 0.025, 0.01, 0.005, 0.0025, 0.001, 0.0005, 0.00025, 0.0001, 0.00005 and 0.00001.

20. The method of claim 1, wherein the concentration of the protein in the patient's sample is lower than a multiplicity factor of the concentration of the protein in the urine from an untreated HIV-positive control human, wherein the patient is identified as having controlled HIV infection, whereby the patient continues to be prescribed the first anti-HIV medication.

21. The method of claim 20, wherein the multiplicity factor is selected from the group consisting of about 0.95, 0.9, 0.8, 0.7, 0.6, 0.5, 0.4, 0.3, 0.2, 0.1, 0.05, 0.025, 0.01, 0.005, 0.0025, 0.001, 0.0005, 0.00025, 0.0001, 0.00005 and 0.00001.

22. The method of claim 1, wherein the at least one protein has an accession number selected from the group consisting of Q8TD57 (SEQ ID NO:1), Q18PE1 (SEQ ID NO:2), Q8NFH5 (SEQ ID NO:3), Q8WYL5 (SEQ ID NO:4), Q8IYD8 (SEQ ID NO:5), 014654 (SEQ ID NO:6), Q96AP4 (SEQ ID NO:7), Q9UQ35 (SEQ ID NO:8), Q8N6W0 (SEQ ID NO:9), Q9H792 (SEQ ID NO:10), Q9H497 (SEQ ID NO:11), Q9UE35 (SEQ ID NO:12), 000743 (SEQ ID NO:13), Q8WXF8 (SEQ ID NO:14), P81274 (SEQ ID NO:15), Q8NG08 (SEQ ID NO:16), Q96AE7 (SEQ ID NO:17), Q9BZM4 (SEQ ID NO:18), Q5T2D3 (SEQ ID NO:19), Q8IXT5 (SEQ ID NO:20), Q9P225 (SEQ ID NO:21), and Q9Y2I9 (SEQ ID NO:22).

23. The method of claim 1, wherein the at least one protein has an accession number selected from the group consisting of P41222 (PTGDS) (SEQ ID NO:23), P14151 (SELL) (SEQ ID NO:24), Q06418 (TYRO3) (SEQ ID NO:25), P52306 (RAP1GDS1) (SEQ ID NO:26), and Q9Y5Y7 (LYVE1) (SEQ ID NO:27).

24. A kit for assessing or monitoring systemic HIV viral load in an HIV-infected human patient, the kit comprising
an antibody or aptamer that binds to at least one protein with an accession number selected from the group consisting of Q8TD57 (SEQ ID NO:1), Q18PE1 (SEQ ID NO:2), Q8NFH5 (SEQ ID NO:3), Q8WYL5 (SEQ ID NO:4), Q8IYD8 (SEQ ID NO:5), O14654 (SEQ ID NO:6), Q96AP4 (SEQ ID NO:7), Q9UQ35 (SEQ ID NO:8), Q8N6W0 (SEQ ID NO:9), Q9H792 (SEQ ID NO:10), Q9H497 (SEQ ID NO:11), Q9UE35 (SEQ ID NO:12), O00743 (SEQ ID NO:13), Q8WXF8 (SEQ ID NO:14), P81274 (SEQ ID NO:15), Q8NG08 (SEQ ID NO:16), Q96AE7 (SEQ ID NO:17), Q9BZM4 (SEQ ID NO:18), Q5T2D3 (SEQ ID NO:19), Q8IXT5 (SEQ ID NO:20), Q9P225 (SEQ ID NO:21), and Q9Y2I9 (SEQ ID NO:22);
an applicator; and,
an instructional material for the use of the kit, wherein the instruction material comprises instructions for analyzing a test sample comprising urine from the patient for the presence or concentration of the at least one protein.

25. The kit of claim 24, further comprising a test data set with a control data set relating to the presence or concentration of the at least one protein in a control sample.

26. The kit of claim 25, wherein the control sample comprises an urine sample from at least one selected from the group consisting of: an untreated HIV-infected control human, an HIV-uninfected control human, and an HIV-infected control human with controlled infection.

27. (canceled)

28. (canceled)

29. A kit for assessing or monitoring systemic HIV viral load in an HIV-infected human patient, the kit comprising
an antibody or aptamer that binds to at least one protein with an accession number selected from the group consisting of P41222 (PTGDS) (SEQ ID NO:23), P14151 (SELL) (SEQ ID NO:24), Q06418 (TYRO3) (SEQ ID NO:25), P52306 (RAP1GDS1) (SEQ ID NO:26), and Q9Y5Y7 (LYVE1) (SEQ ID NO:27);
an applicator; and,
an instructional material for the use of the kit, wherein the instruction material comprises instructions for analyzing a test sample comprising urine from the patient for the presence or concentration of the at least one protein.

30. The kit of claim 29, further comprising a test data set with a control data set relating to the presence or concentration of the at least one protein in a control sample.

31. The kit of claim 30, wherein the control sample comprises an urine sample from at least one selected from the group consisting of: an untreated HIV-infected control human, an HIV-uninfected control human, and an HIV-infected control human with controlled infection.

32. (canceled)

33. (canceled)
